(19) United States
(12) Patent Application Publication
(10) Pub. No.: US 2025/0381206 A1
(43) Pub. Date: Dec. 18, 2025

(54) INVARIANT NATURAL KILLER T-CELL ACTIVATORS

(71) Applicant: Deciduous Therapeutics, Inc., San Francisco, CA (US)

(72) Inventors: Scott ARMSTRONG, South San Francisco, CA (US); Thomas BOBINSKI, South San Francisco, CA (US); Anil BHUSHAN, Oakland, CA (US); Matthew DUNCTON, San Bruno, CA (US); Rachel HATANO, South San Francisco, CA (US); Leah MAKLEY, South San Francisco, CA (US); Robin MANSUKHANI, South San Francisco, CA (US)

(73) Assignee: Deciduous Therapeutics, Inc., San Francisco, CA (US)

(21) Appl. No.: 19/107,539

(22) PCT Filed: Aug. 31, 2023
(86) PCT No.: PCT/US2023/031720

§ 371 Date: Feb. 27, 2025

Related U.S. Application Data
(60) Provisional application No. 63/402,921, filed on Aug. 31, 2022.

(51) Int. Cl. A61K 31/7034; A61K 31/7042; A61P 37/04; C07H 15/20; C07H 15/26

(52) U.S. Cl. CPC A61K 31/7034; A61K 31/7042; A61P 37/04; C07H 15/20; C07H 15/26

(57) ABSTRACT

Disclosed herein are compounds of the formulas (A-I) and (B) that may be useful for activating invariant natural killer T (iNKT) cells. According to certain disclosed embodiments activation of iNKT cells induces production of one or more cytokines, such as IFNγ, IL-2, IL-4, IL-6 and TNFα, and results in reduction of senescent cells.

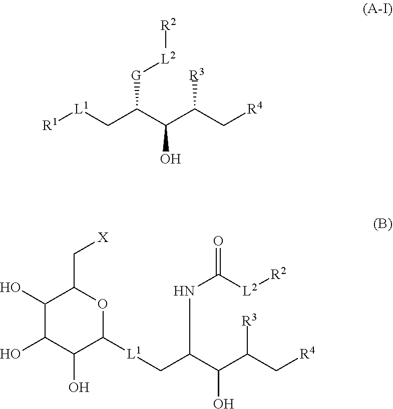

### CROSS-REFERENCE TO RELATED APPLICATIONS

This application claims priority to and benefit of U.S. Provisional Application Ser. No. 63/402,921, filed Aug. 31, 2022, which is incorporated by reference in its entirety.

### FIELD

The present disclosure relates generally to activating invariant natural killer T (INKT) cells, and more specifically to compounds that may be useful for activating iNKT cells, which induces production of one or more cytokines, such as IFNγ, IL-2, IL-4, IL-6 and TNFα, and results in reduction of senescent cells.

### BACKGROUND

In a healthy system, the immune system naturally (endogenously) clears senescent cells. When this immune function is compromised, senescent cells build up and can propagate into a multitude of different diseases. Invariant natural killer T (INKT) cells are a subset of T cells that recognize glycolipid antigens bound to the cluster of differentiation (CD) 1d molecule expressed by surface antigen presenting cells. Recognition of exogenous and endogenous lipids can aid in immune response to maladies such as autoimmune disease, allergic disease, metabolic syndrome, cancer and pathogen infection. Although iNKT cells have been shown to mediate immune responses based on cytokine release, iNKT cells can also function as effectors by cell cytotoxicity.

### BRIEF SUMMARY

In some aspects, disclosed herein are compounds for activating invariant natural killer T (INKT) cells. In one embodiment, activation of iNKT cells results in reduction of senescent cells. According to certain disclosed embodiments activation of iNKT cells induces production of one or more cytokines, such as IFNγ, IL-2, IL-4, IL-6 and TNFα. In some embodiments, activated iNKT cells are used to selectively reduce the presence of inflammatory senescent cells, such as senescent cells having an inflammatory secretome (SASP). Methods for activating iNKT cells by contacting an iNKT cell with an amount of a compound disclosed herein are also described. In one embodiment, contacting the iNKT cells indirectly selectively reduces the presence of senescent cells. Pharmaceutical compositions for practicing the disclosed methods also are described.

In some embodiments, disclosed compounds are represented by the formula

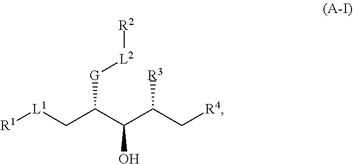

wherein variable groups are as herein described.

In some embodiments, disclosed compounds are represented by the formula

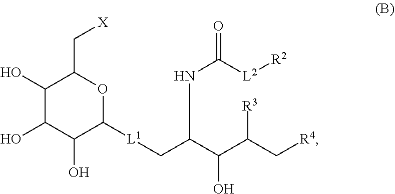

wherein variable groups are as herein described.

In some embodiments, disclosed compounds are represented by the formula

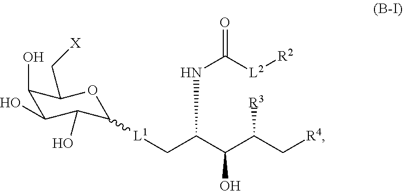

wherein variable groups are as herein described.

### DETAILED DESCRIPTION

The following description sets forth exemplary compositions, methods, parameters and the like. It should be recognized, however, that such description is not intended as a limitation on the scope of the present disclosure but is instead provided as a description of exemplary embodiments.

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

Compounds herein can include all stereoisomers, enantiomers, diastereomers, mixtures, racemates, atropisomers, and tautomers of the structures illustrated.

In some variations, “acyl” includes the groups HC(O)—, alkyl-C(O)—, cycloalkyl-C(O)—, cycloalkenyl-C(O)—, aryl-C(O)—, heteroaryl-C(O)— and heterocyclyl-C(O)-where alkyl, cycloalkyl, cycloalkenyl, aryl, heteroaryl, and heterocyclyl are as described herein. In one variation, by way of example, acyl groups include acetyl and benzoyl groups.

In some variations, “alkyl” refers to an optionally substituted straight-chain, or optionally substituted branched-chain saturated hydrocarbon, wherein an sp³-hybridized carbon of the alkyl residue is attached to the rest of the molecule by a single bond. In one variation, non-limiting examples of alkyl groups include straight, branched and alkylene groups.

In some variations, “alkylene” or “alkylene chain” refers to a branched or unbranched divalent saturated hydrocarbon chain, linking the rest of the molecule to a radical group, and in some variations, having from 1 to 40 carbon atoms, such as from 2 to 20 carbon atoms, such as from 7 to 15 carbon atoms, or from 1 to 6 carbon atoms. In one variation, this term is exemplified by groups such as methylene (—CH₂—), ethylene (—CH₂CH₂—), the propylene isomers (e.g., —CH₂CH₂CH₂— and —CH(CH₃)CH₂—) and the like. In certain variations, further examples may include lipid chains. An alkyl group or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

In certain variations, alkyl groups may include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, tridecyl, pentadecyl, octadecyl, and the like. Whenever it appears herein, a numerical range such as “C₁₋₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₂₋₂₀ alkyl, C₇₋₁₅ alkyl, C₁₋₁₀ alkyl, a C₁₋₉ alkyl, a C₁₋₈ alkyl, a C₁₋₇ alkyl, a C₁₋₆ alkyl, a C₁₋₅ alkyl, a C₁₋₄ alkyl, a C₁₋₃ alkyl, a C₁₋₂ alkyl, or a C₁ alkyl. Unless stated otherwise specifically in the specification, in some variations, an alkyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen. In one embodiment, non-limiting examples of substituted alkyl groups include hydroxymethyl, chloromethyl, trifluoromethyl, aminomethyl, 1-chloroethyl, 2-hydroxyethyl, 1,2-difluoroethyl, and 3-carboxypropyl.

In some variations, “cycloalkyl” by itself or as part of another substituent refers to a saturated or unsaturated cyclic alkyl radical. Where a specific level of saturation is intended, the nomenclature “cycloalkanyl” or “cycloalkenyl” is used. In some variations, cycloalkyl groups also include fused-, bridged-, and spiro-bicycles and higher fused-, bridged-, and spiro-systems. In certain variations, a cycloalkyl group can be substituted with any number of straight, branched, or cyclic alkyl groups. In one variation, non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3] undecanyl. In certain embodiments, the cycloalkyl group is C₃₋₁₀ cycloalkyl, such as C₃₋₇ cycloalkyl.

In some variations, “alkenyl” refers to an optionally substituted straight-chain, or optionally substituted branched-chain hydrocarbon having one or more carbon-carbon double-bonds and having from two to about ten carbon atoms, and in certain variations, having from two to about six carbon atoms, wherein an sp²-hybridized carbon of the alkenyl residue is attached to the rest of the molecule by a single bond. The group may be in either the cis or trans conformation about the double bond(s), and should be understood to include both isomers. In one variation, examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂₋₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂₋₁₀ alkenyl, a C₂₋₉ alkenyl, a C₂₋₈ alkenyl, a C₂₋₇ alkenyl, a C₂₋₆ alkenyl, a C₂₋₅ alkenyl, a C₂₋₄ alkenyl, a C₂₋₃ alkenyl, or a C₂ alkenyl. In some variations, unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

In some variations, non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

In some variations, non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1. 1] hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3] undecanyl.

In some variations, non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Alkenylene refers to a branched or unbranched divalent hydrocarbon chain having at least one alkene in the chain, linking the rest of the molecule to a radical group, and in certain variations, having from 1 to 40 carbon atoms, such as from 2 to 20 carbon atoms, such as from 7 to 15 carbon atoms, or from 1 to 6 carbon atoms. In one variation, non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

In certain variations, non-limiting examples of alkynyl groups include straight, branched, and cyclic alkynyl groups. The triple bond of an alkynyl group can be internal or terminal. An alkynyl or alkynylene group can be, for example, a C₂, C₃, C₄, C₅, C₆, C₇, C₈, C₉, C₁₀, C₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. In one variation, non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A haloalkyl group is any alkyl group substituted with any number of halogen atoms, for example, those selected from fluorine, chlorine, bromine, and iodine atoms. A haloalkenyl group can be any alkenyl group substituted with any number of halogen atoms. A haloalkynyl group can be any alkynyl group substituted with any number of halogen atoms.

In some variations, an alkoxy group can be, for example, an oxygen atom substituted with any alkyl, alkenyl, or alkynyl group. An ether or an ether group comprises an alkoxy group. In one variation, non-limiting examples of alkoxy groups include methoxy, ethoxy, propoxy, isopropoxy, and isobutoxy.

“Halo” or “halogen” refers to bromo, chloro, fluoro, iodo, or bromine, chlorine, fluorine or iodine. In some embodiments, halo is fluoro or chloro. In some embodiments, halo is fluoro.

In some variations, “heteroalkyl” refers to an alkyl group in which one or more skeletal atoms of an alkyl moiety are selected from an atom other than carbon, such as oxygen, nitrogen (for example, —NH—, —N(alkyl)-), sulfur, or combinations thereof. A heteroalkyl is attached to the rest of the molecule at a carbon atom of the heteroalkyl. In one variation, a heteroalkyl is a C₁₋₆ heteroalkyl wherein the heteroalkyl has 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. In one variation, examples of such heteroalkyl are, for example, —CH₂OCH₃, —CH₂CH₂OCH₃, —CH₂CH₂OCH₂CH₂OCH₃, or —CH(CH₃) OCH₃. In certain variations, unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

In some variations, a heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. In one variation, non-limiting examples of heterocycles include carboranes, pyrrole, pyrrolidine, pyridine, pyrimidine, pyrazine, pyridazine, piperidine, succinimide, maleimide, morpholine, imidazole, thiophene, furan, tetrahydrofuran, pyran, and tetrahydropyran.

In other variations, non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1 H-azepinyl, 2,3-dihydro-1 H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1 H-indolyl, 1,2,3,4-tetrahydroquinolinyl, and decahydro-1H-cycloocta [b] pyrrolyl.

In some variations, “heterocyclylalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. In certain variations, unless stated otherwise specifically in the specification, the heterocyclylalkyl radical may be a monocyclic, bicyclic, tricyclic, or tetracyclic ring system, which may include fused (when fused with an aryl or a heteroaryl ring, the heterocyclylalkyl is bonded through a non-aromatic ring atom) or bridged ring systems; and the nitrogen, carbon, or sulfur atoms in the heterocyclylalkyl radical may be optionally oxidized; the nitrogen atom may be optionally quaternized.

In some variations, representative heterocyclylalkyls include, but are not limited to, heterocyclylalkyls having from two to fifteen carbon atoms (C₂₋₁₅ heterocyclylalkyl), from two to ten carbon atoms (C₂₋₁₀ heterocyclylalkyl), from two to eight carbon atoms (C₂₋₈ heterocyclylalkyl), from two to six carbon atoms (C₂₋₆ heterocyclylalkyl), from two to five carbon atoms (C₂₋₅ heterocyclylalkyl), or two to four carbon atoms (C₂₋₄ heterocyclylalkyl). In some embodiments, the heterocyclylalkyl is a 3- to 6-membered heterocyclylalkyl. In some embodiments, the heterocyclylalkyl is a 5- to 6-membered heterocyclylalkyl. In one variation, examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). In certain variations, unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen.

In some variations, “heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocyclylalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b] [1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1 H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

“Substituted” refers to a group in which one or more hydrogen atoms are independently replaced with the same or different substituent(s). In some variations, non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, haloalkyl groups, alkenyl groups, haloalkenyl groups, alkynyl groups, haloalkynyl groups, alkoxy groups, aryl groups, aryloxy groups, aralkyl groups, arylalkoxy groups, heterocyclylalkyl groups, heteroaryl groups, cycloalkyl groups, acyl groups, acyloxy groups, carbamate groups, amide groups, ureido groups, epoxy groups, and ester groups.

“Pharmaceutically acceptable carrier” refers to a diluent, adjuvant, excipient or vehicle with, or in which a compound is administered.

“Treating” or “treatment” of any condition, such as an autoimmune, metabolic, allergic, cancer or infectious disease, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition. In some variations, “treating” or “treatment”, as used herein, refer to a method or procedure for obtaining beneficial or desired results—for example, clinical results. Beneficial or desired results may include: (1) alleviating one or more symptoms caused by or associated with a disease, disorder, or condition; (2) reducing the extent of the disease, disorder, or condition; (3) slowing or stopping the development or progression of one or more symptoms caused by or associated with the disease, disorder, or condition (for example, stabilizing the disease, disorder, or condition); and (4) relieving the disease, for example, by causing the regression of one or more clinical symptoms (e.g., ameliorating the disease state, enhancing the effect of another medication, delaying or stopping the progression of the disease, increasing the quality of life, and/or prolonging survival rates).

“Therapeutically effective amount” means the amount of a compound that, when administered to a patient for preventing or treating a condition such as an autoimmune, metabolic, allergic, cancer or infectious disease, is sufficient to effect such treatment. In some variations, “therapeutically effective” applied to dose or amount refers to that quantity of a compound or pharmaceutical formulation, such as those described elsewhere herein, that is sufficient to result in a desired clinical benefit after administration to a patient in need thereof. It is to be understood that an effective amount may be in one or more doses, e.g., a single dose or multiple doses may be needed to achieve the desired treatment endpoint. The “therapeutically effective amount” will vary depending on the compound, the condition and its severity and the age, weight, etc., of the patient.

### II. Compounds

In some aspects, provided herein are compounds that may act as iNKT activators. Compounds of Formulas (A-I)-(A-IX)

In one embodiment, compounds disclosed herein have Formula (A-I)

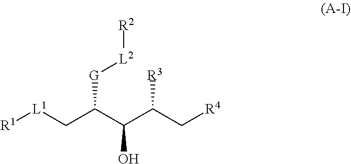

or a pharmaceutically acceptable salt thereof, wherein:
R¹ is selected from

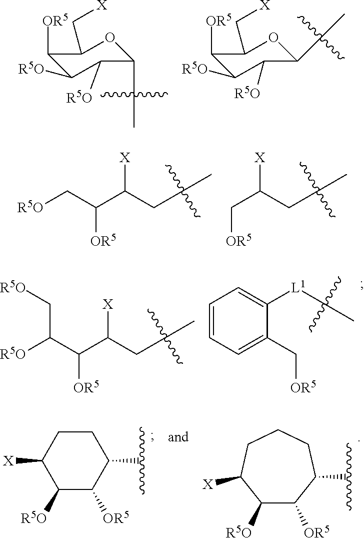

R² is selected from —Si(R^{a})₄, cycloalkyl and heterocyclylalkyl, wherein the cycloalkyl and heterocyclylalkyl groups are optionally substituted with one or more R^{a}, R^{b}, R^{d} substituted with one or more of the same or different R^{b}, and —OR^{a} substituted with one or more of the same or different R^{b};
R³ is hydrogen or —OR^{a};
R⁴ is C₈-C₁₅ alkyl;
R⁵ is, for each occurrence, independently selected from hydrogen, C₁₋₆ alkyl, —C(O)OR^{a}, —C(O)R^{a}, —C(O)NR^{c}R^{c};
X is selected from —OH, —OC(O)OR⁶, —OC(O)R⁷, —OC(O)OR⁸, —OC(O)NR⁹R¹⁰; —SR¹¹, —S(O)₂R¹²; —NR⁹R¹⁰; —NC(O)R¹³, —NC(O)OR¹⁴, —NC(O)NR⁹R¹⁰, 5-membered heterocyclyl, and 5-membered heteroaryl;
L¹ is —O—; —(CH₂)ₘ—; —S—, —S(O)₂—; —N(H)—; —O—(CH₂)ₘ—; or —(CH₂)ₘ—O—;
G is selected from —NR^{a}C(O)—; N(R^{a})C(R^{c})₂—; —C═C(R^{b})—; —NS(O) (R^{a})—; 5-membered heterocyclylalkyl, and 5-membered heteroaryl;
L² is a C₈-C₃₀ alkylene or alkenylene chain, optionally having one or more methylene replaced with —O—, —S—, —S(O)₂—, —N(H)—, —N(R^{L})— and Si(R^{c})₂—; and optionally having two or more adjacent methylene groups linked by a cyclopropyl moiety;
R^{a} is, for each occurrence, independently selected from hydrogen and C₁₋₆ alkyl;
R^{b} is, for each occurrence, independently selected from halogen and —CF₃,
R^{c} is, for each occurrence, hydrogen, alkyl, haloalkyl, or two Re together with the atom to which they are attached form a heterocyclylalkyl ring that is unsubstituted or substituted with one or more halogen, C₁₋₆ alkyl, C(O)OR^{a}, —C(O)R^{a};
R^{d} is, for each occurrence, acyl, alkyl, heteroalkyl, cycloalkyl, heterocyclylalkyl, aryl, heteroaryl, each optionally substituted with one or more R^{B};
R^{L} is, for each occurrence, selected from C₁₋₁₅ alkyl, cycloalkyl and heterocyclylalkyl; m is, for each occurrence, 1 or 2;
each of R⁶, R⁷, R⁸, R⁹, R¹¹, R¹², R¹³, and R¹⁴ is independently hydrogen, alkyl, alkenyl, haloalkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A},
each of R⁹ and R¹⁰ is independently alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A}, or R⁹ and R¹⁰ together with the atom to which they are attached form a heterocyclylalkyl ring or a heteroaryl ring that is unsubstituted or substituted with one or more R^{A};
each R^{A} is independently alkyl, heteroalkyl, cycloalkyl, heterocyclylalkyl, aryl, heteroaryl, oxo, —OR¹⁵, —N(R¹⁶)R¹⁷, —C(O)OR¹⁵, —N(R^{a})C(O)OR¹⁵, —N(R^{a})C(O)R¹⁵, —C(O)R¹⁵, —OC(O)R¹⁵, —OC(O)OR¹⁵, —C(O)N(R¹⁶)R¹⁷, —OC(O)N(R¹⁶)R¹⁷, wherein alkyl, heteroalkyl, cycloalkyl, heterocyclylalkyl, aryl, or heteroaryl is unsubstituted or substituted with alkyl, halogen, oxo, —OR^{a}, —NR(R¹⁶)R¹⁷, —C(O)R¹⁵, —OC(O)R¹⁵, —OC(O)OR¹⁵, or —OC(O)N(R¹⁶)R¹⁷;
each R¹⁵ is independently alkyl, heteroalkyl, cycloalkyl, or hydrogen, wherein alkyl, heteroalkyl, cycloalkyl is unsubstituted or substituted with one or more R^{B};
each of R¹⁶ and R¹⁷ is independently alkyl, or hydrogen, wherein alkyl, is unsubstituted or substituted with one or more R^{B}; or R¹⁶ and R¹⁷ together with the atom to which they are attached form a heterocyclylalkyl ring or heteroaryl ring, each of which is unsubstituted or substituted with one or more R^{B};
each R^{B} is independently halogen, amino, cyano, hydroxyl, alkyl, cycloalkyl, acyl wherein each cycloalkyl and acyl is unsubstituted or substituted with one or more halogen, amino, cyano, hydroxyl, alkyl, acetyl, or benzoyl;
provided that, when X is OH, L¹ is —(CH₂)ₘ—; —S—, —S(O)₂—; —N(H)—; —O—(CH₂)ₘ—; or —(CH₂)ₘ—O—.

In one embodiment, compounds disclosed herein have Formula (A-II)

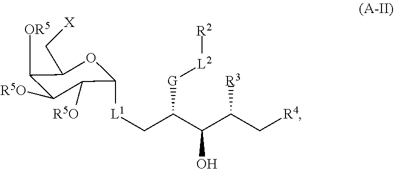

or a pharmaceutically acceptable salt thereof.

In one embodiment, compounds disclosed herein have Formula (III)

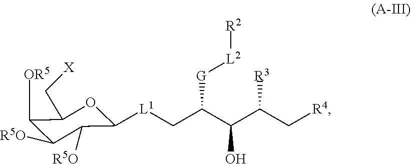

or a pharmaceutically acceptable salt thereof.

In one embodiment, compounds disclosed herein have Formula (A-IV)

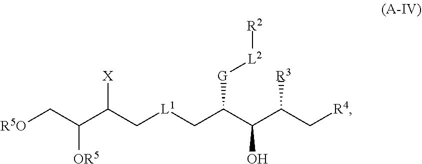

or a pharmaceutically acceptable salt thereof.

In one embodiment, compounds disclosed herein have Formula (A-V)

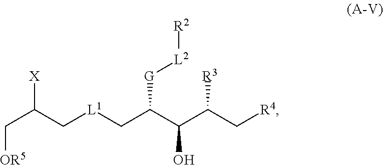

or a pharmaceutically acceptable salt thereof.

In one embodiment, compounds disclosed herein have Formula (A-VI)

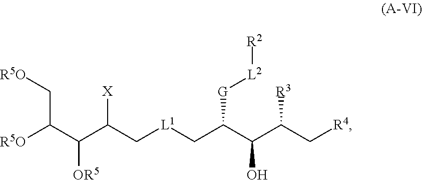

or a pharmaceutically acceptable salt thereof.

In one embodiment, compounds disclosed herein have Formula (A-VII)

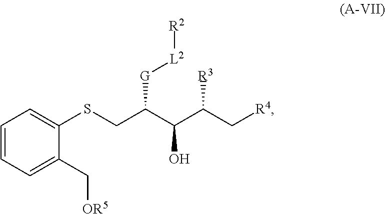

or a pharmaceutically acceptable salt thereof.

In one embodiment, compounds disclosed herein have Formula (A-VIII)

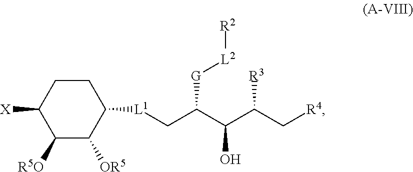

or a pharmaceutically acceptable salt thereof.

In one embodiment, compounds disclosed herein have Formula (A-IX)

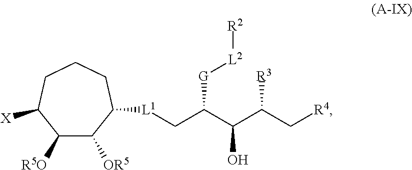

or a pharmaceutically acceptable salt thereof.

In one embodiment of compounds disclosed herein, including compounds of Formulas (A-I), (A-II) and (A-III), X is a 5-membered heterocyclyl. In certain aspects of Formulas (A-I), (A-II) and (A-III), X is selected from

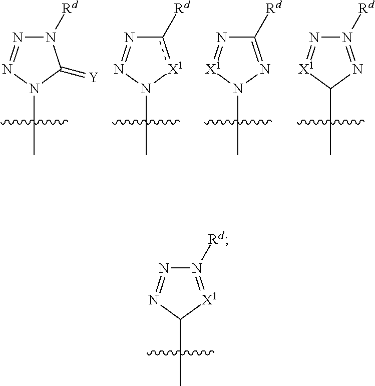

wherein
Y is O or S; and
X¹ is selected from —N—, —CH—, —C(R^{d})— and —C(R⁴)₂—.

In embodiments of compounds disclosed herein, including compounds of Formulas (A-I)-(A-IX), L¹ is selected from —(CH₂)ₘ—; —S—, —S(O)₂—; —N(H)—; —O—(CH₂)ₘ—; or —(CH₂)ₘ—O—. In such compounds m is, for each occurrence, 1 or 2. In certain embodiments of compounds according to Formulas (A-I)-(A-IX), L¹ is —O—. In additional embodiment of Formulas (A-I)-(A-IX), L¹ is —(CH₂)—.

In embodiments of compounds disclosed herein, wherein the compound has a structure of Formula (A-I), (A-II), (A-III), (A-IV), (A-V), (A-VI), (A-VII), (A-VIII) or (A-IX), G is selected from

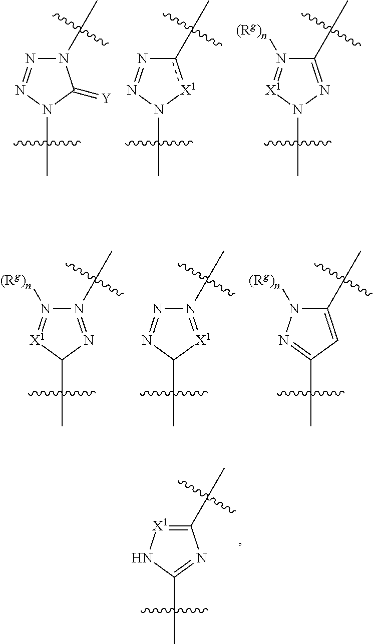

wherein
Y is O or S;
X¹ is selected from —N—, —CH—, —C(R^{d})— and —C(R^{d})₂—;
n is 0 or 1; and
R^{g} is hydrogen, C₁₋₆ alkyl, —C(O)OR^{a}, —C(O)R^{a}, or —C(O)NR^{c}R^{c}.

In particular examples of compounds according to Formulas (A-I)-(A-IX), G is selected from

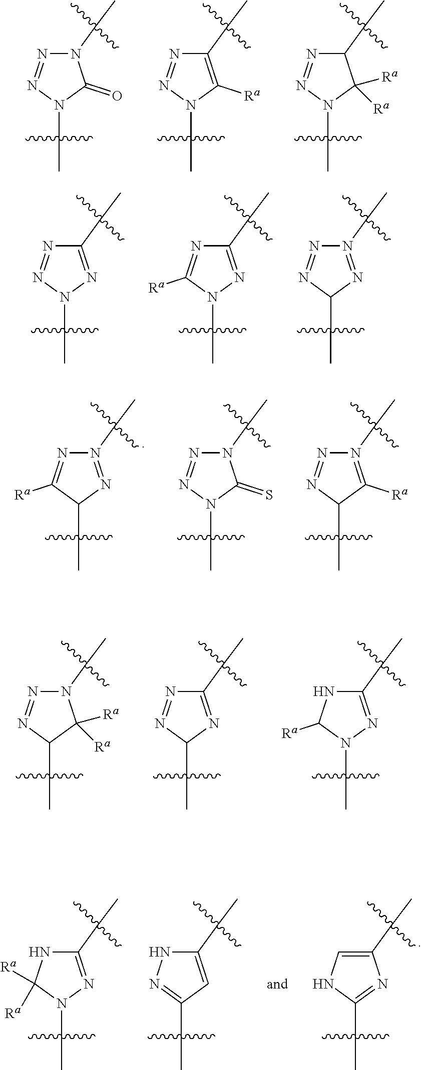

In other examples of compounds according to Formulas (A-I)-(A-IX), G is selected from —NRC(O)—; —N(R^{a})C(R^{c})₂—; —C═C(R^{b})—; —NS(O) (R^{a})—. In one such embodiment, a compound has G being-C═C(R^{b})—, wherein R^{b} is halo. In certain such examples of compounds according to Formulas (A-I)-(A-IX), G is —C═C(R^{b})—, wherein R^{b} is fluoro.

In embodiments of compounds according to Formulas (A-I)-(A-IX), G is —N(R^{a})C(R^{c})₂—. In certain examples of compounds according to Formulas (A-I)-(A-IX), G is —N(R^{a})C(R^{c})₂—, wherein one Re is trifluoromethyl and the other is hydrogen. In other examples of compounds according to Formulas (A-I)-(A-IX), G is —N(R^{a})C(R^{c})₂—, wherein two Re together with the carbon atom to which they are attached form a heterocyclylalkyl ring, and in particular examples the heterocyclylalkyl ring formed by two Re together with the carbon atom to which they are attached is an oxetane ring.

In embodiments of compounds according to Formulas (A-I)-(A-IX), L² is C₈-C₃₀ alkylene or alkenylene chain, optionally having one or more methylene replaced with —O—, —S—, —S(O)₂—, —N(H)—, —N(R^{L})—, —Si(R^{c})₂—; and optionally having two or more adjacent methylene groups linked by a cyclopropyl moiety. In certain embodiments of compounds according to Formulas (A-I)-(A-IX), L² is C₈-C₃₀ alkylene or alkenylene chain, having one methylene replaced with —O—, —S—, —S(O)₂—, —N(H)— or —N(R^{L})—. In one embodiment, a compound of L² is C₈-C₃₀ alkylene having two or more methylenes replaced with —O—, —S—, —S(O)₂—, —N(H)—, —N(R^{L})— or a combination thereof. In one embodiment, a compound has Formula (A-I), (A-II), (A-III), (A-IV), (A-V), (A-VI), (A-VII), (A-VIII) and/or (A-IX) L² is C₈-C₃₀ alkylene or alkenylene chain, having one methylene replaced with —N(R^{L})—. In certain such embodiments wherein L² is C₈-C₃₀ alkylene or alkenylene chain having one methylene replaced with —N(R^{L})—, R^{L} is selected from C₁₋₁₅ alkyl, C₃-8 cycloalkyl and C₂₋₇ heterocyclylalkyl. In one such embodiment, R^{L} is C₃₋₈ cycloalkyl, such as bicyclo[1.1.1]pentanyl. In another embodiment of compounds wherein L² is C₈-C₃₀ alkylene or alkenylene chain, having one methylene replaced with —N(R^{L})—, R^{L} is C₁₋₁₅ alkyl, such as C₁₋₆ alkyl or C₃₋₁₀ alkyl that is substituted or unsubstituted. In one such embodiment wherein R^{L} is C₁₋₁₅ alkyl, R^{L} is unsubstituted C₁₋₁₅ alkyl, such as —C₆H₁₃, —C₇H₁₅, —C₈H₁₇ or —C₁₅H₃₁. In embodiments of compounds according to Formulas (A-I)-(A-IX), L² is C₈-C₃₀ alkylene or alkenylene chain at least one cyclopropanyl. In embodiments of compounds according to Formulas (A-I)-(A-IX), L² is C₁₀-C₂₀ alkylene, such as C₁₀-C₁₅ alkylene.

In embodiments of compounds according to Formulas (A-I)-(A-IX), R² is selected from

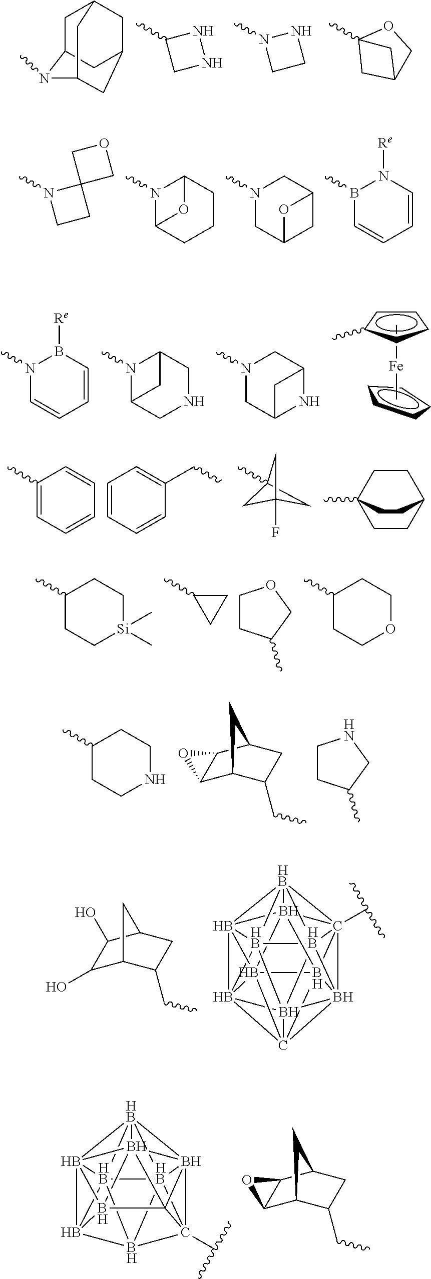

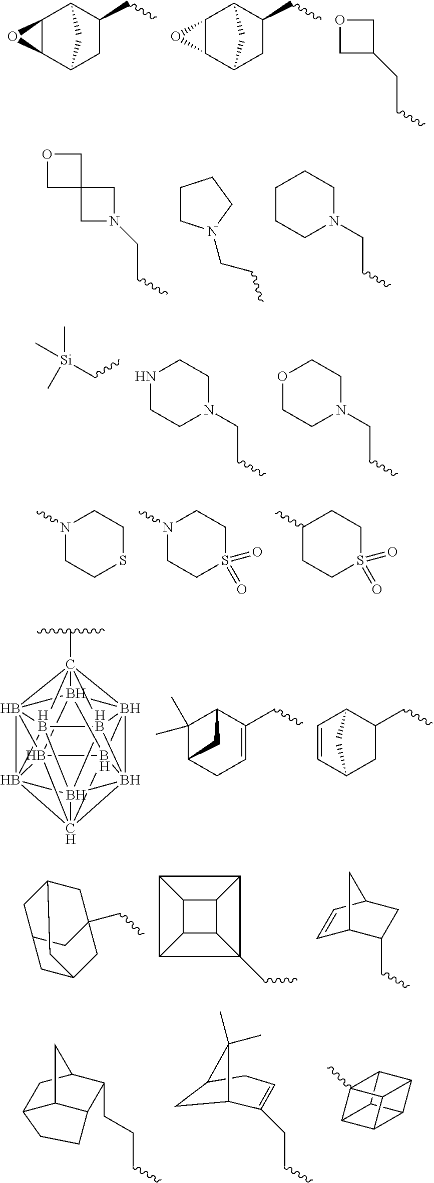

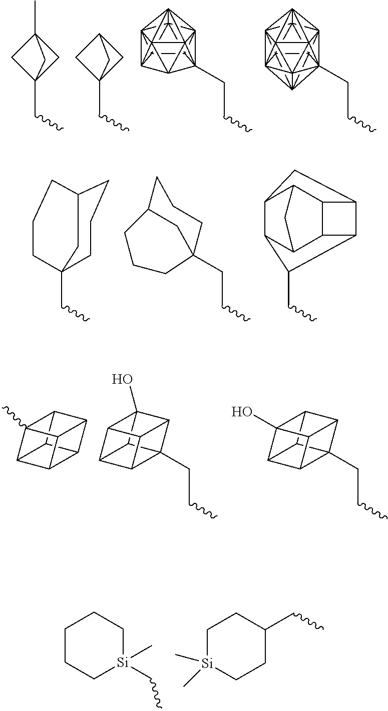

wherein [symbol] indicates the bond to L² and each R² is optionally substituted with one or more R^{e}, wherein R^{e} is, for each occurrence, selected from halogen, C₁₋₁₅ alkyl, C₁₋₆ haloalkyl, —C(O)OR^{a}, —C(O)R^{a} and —C(O)NR^{c}R^{c}.

In some embodiments of compounds according to Formulas (A-I)-(A-IX), R² is selected from —OR^{a}, —SR^{a}, —NR^{c}R^{c} and haloalkyl. In some embodiments of compounds according to Formulas (A-I)-(A-IX), R² is haloalkyl.

In certain embodiments of compounds according to Formulas (A-I)-(A-IX), R² is selected from

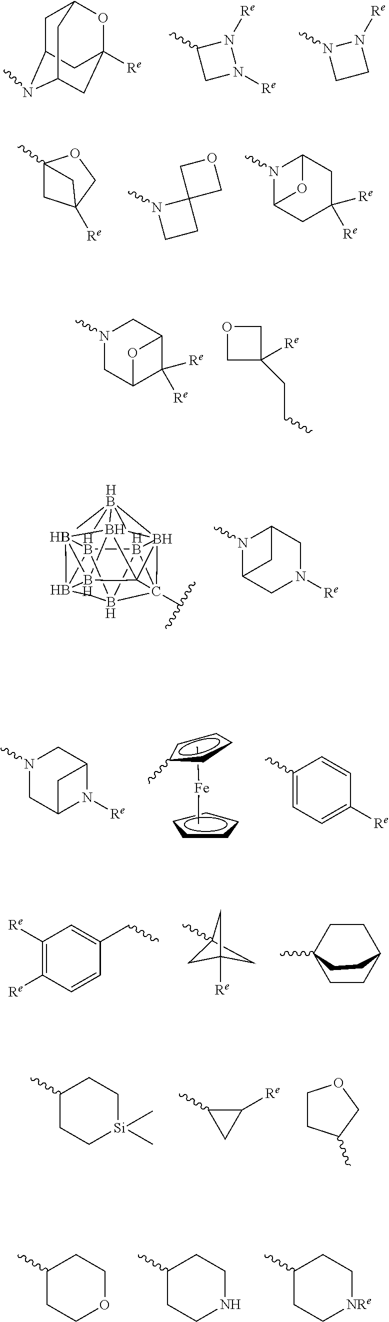

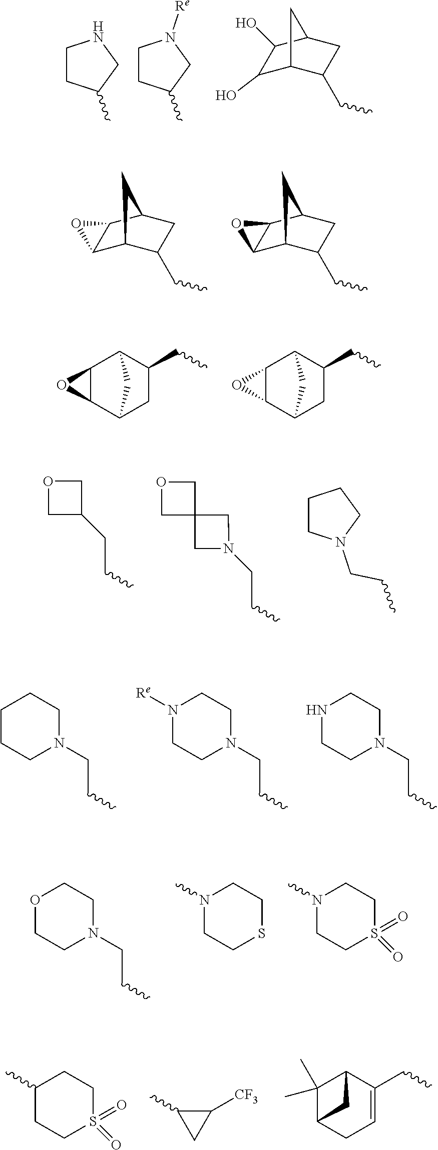

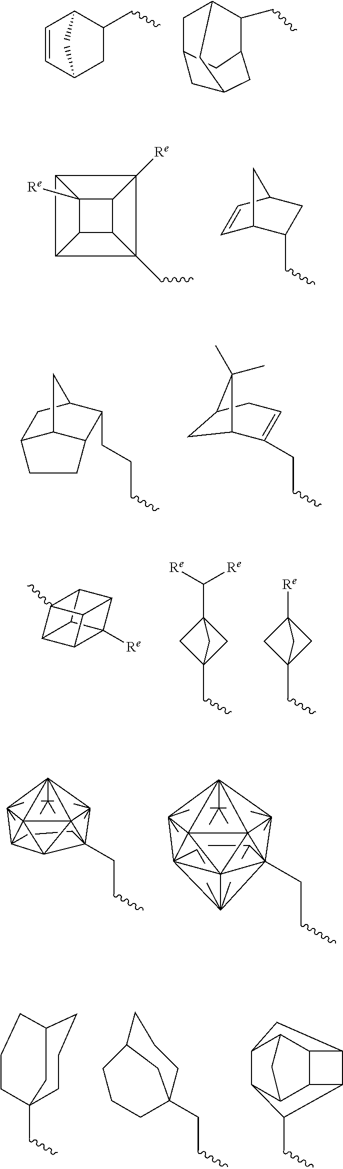

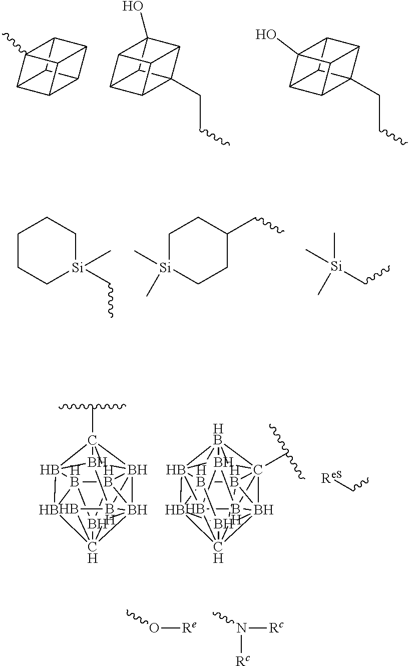

In certain embodiments of compounds according to Formulas (A-I)-(A-IX), R² is selected from

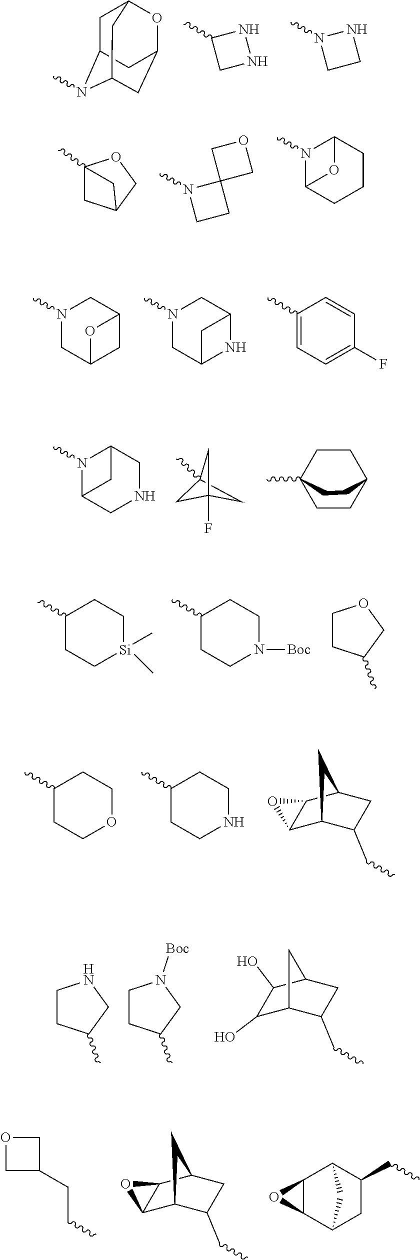

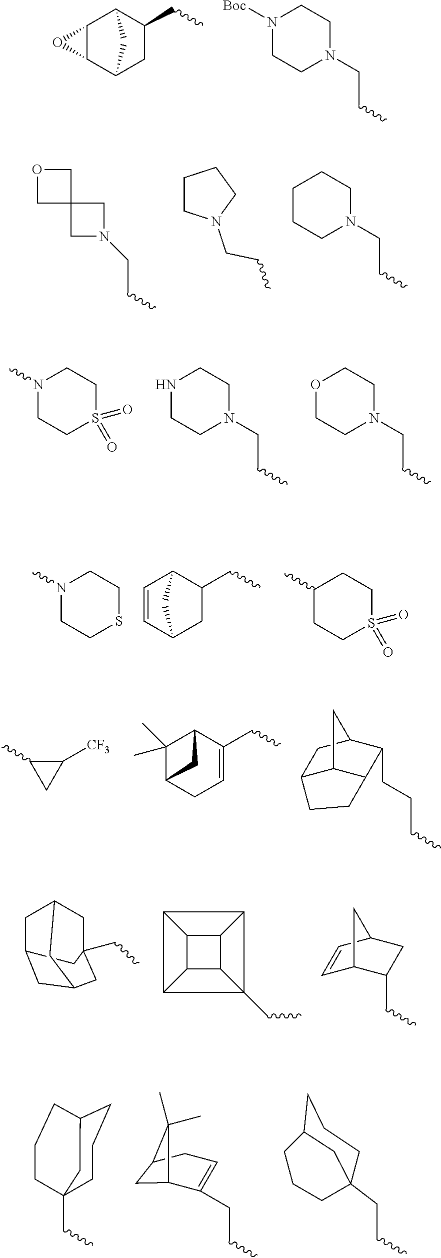

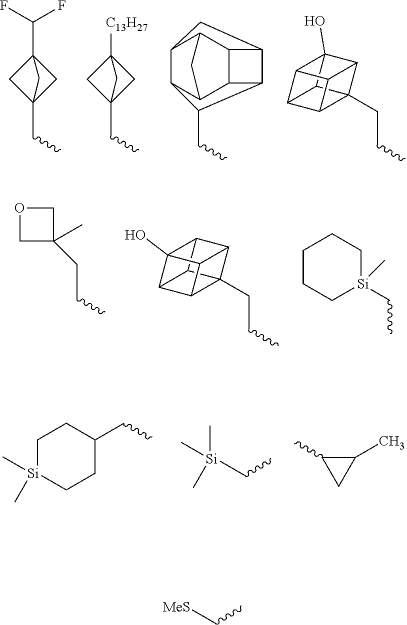

In one embodiment of compounds according to Formulas (A-I)-(A-IX), R³ is hydrogen. In another embodiment of compounds according to Formulas (A-I)-(A-IX), R³ is —OH.

In particular embodiments of compounds according to Formulas (A-I)-(A-IX), R⁴ is C₈₋₁₅ alkyl, such as in compounds wherein R⁴ is —C₁₂H₂₅, —C₁₃H₂₇, or —C₁₄H₂₉. In certain embodiments of compounds according to Formulas (A-I)-(A-IX), R⁴ is —C₁₃H₂₇.

In particular embodiments of compounds according to Formulas (A-I)-(A-IX), R⁵ is selected from hydrogen, C₁₋₆ alkyl, —C(O)OR^{a}, —C(O)R^{a}, —C(O)NR^{c}R^{c}. In some embodiments of compounds according to Formulas (A-I)-(A-IX), at least one R⁵ is hydrogen, and in certain such embodiments, each R⁵ is hydrogen.

### Compounds of Formulas (B) and (B-I)-(B-IV)

In some aspects, provided is a compound of Formula (B):

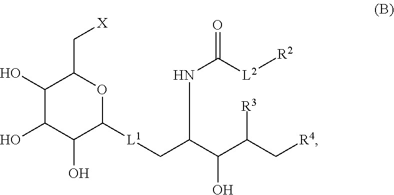

or a pharmaceutically acceptable salt thereof, wherein:
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —O— or —(CH₂)ₘ—;
L² is alkylene;
X is —OH, —OC(O)R⁷, —OC(O)NR⁹R¹⁰, or —N(R^{a})C(O)R¹³, wherein
   when X is OH or —N(R^{a})C(O)R¹³, either
      L¹ is —(CH₂)ₘ—, or
      R³ is hydrogen;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl, wherein the cycloalkyl is optionally substituted with one or more halo;
R¹³ is cycloalkyl optionally substituted with one or more halo; and
each R^{A} is independently aryl.

In one aspect, provided herein is a compound of Formula (B-I):

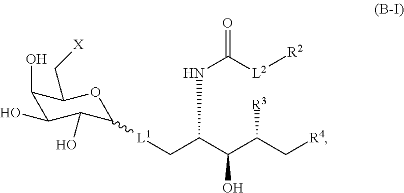

or a pharmaceutically acceptable salt thereof, wherein:
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —O— or —(CH₂)ₘ—;
L² is alkylene;
X is —OH, —OC(O)R⁷, —OC(O)NR⁹R¹⁰, or —N(R^{a})C(O)R¹³, wherein
   when X is OH or —N(R^{a})C(O)R¹³, either
      L¹ is —(CH₂)ₘ—, or
      R³ is hydrogen;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl, wherein the cycloalkyl is optionally substituted with one or more halo;
R¹³ is cycloalkyl; and
each R^{A} is independently aryl.

In some embodiments of a compound of Formula (B) or (B-I), or a pharmaceutically acceptable salt thereof,
R² is cycloalkyl or heterocyclylalkyl, wherein the cycloalkyl or heterocyclylalkyl is optionally substituted with one or more R^{b};
R³ is hydrogen or —OH;
R⁴ is C₈-C₁₅ alkyl;
L¹ is —O— or —(CH₂)ₘ—;
L² is C₈-C₃₀ alkylene;
X is —OH, —OC(O)R⁷, —OC(O)NR⁹R¹⁰, or —N(R^{a})C(O)R¹³, wherein
   when X is OH or —N(R^{a})C(O)R¹³, either
      L¹ is —(CH₂)ₘ—, or
      R³ is hydrogen;
R^{a} is, for each occurrence, independently hydrogen or C₁₋₆ alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl;
R¹³ is cycloalkyl; and
each R^{A} is independently aryl.

In some embodiments of a compound of Formula (B) or (B-I), or a pharmaceutically acceptable salt thereof,
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —(CH₂)ₘ—;
L² is alkylene;
X is —OH or —N(R^{a})C(O)R¹³;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl;
R¹³ is cycloalkyl; and
each R^{A} is independently aryl.

In some embodiments of a compound of Formula (B) or (B-I), or a pharmaceutically acceptable salt thereof,
R² is cycloalkyl or heterocyclylalkyl, wherein the cycloalkyl or heterocyclylalkyl is optionally substituted with one or more R^{b};
R³ is hydrogen;
R⁴ is alkyl;
L¹ is —O— or —(CH₂)ₘ—;
L² is alkylene;
X is —OH or —N(R^{a})C(O)R¹³;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl;
R¹³ is cycloalkyl; and
each R^{A} is independently aryl.

In some embodiments of a compound of Formula (B) or (B-I), or a pharmaceutically acceptable salt thereof,
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —O— or —(CH₂)ₘ—;
L² is alkylene;
X is —OC(O)R⁷ or —OC(O)NR⁹R¹⁰;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R^{c} and R¹⁰ is independently hydrogen or cycloalkyl; and
each R^{A} is independently aryl.

In one aspect, provided herein is a compound of Formula (B-I-i) or (B-I-ii):

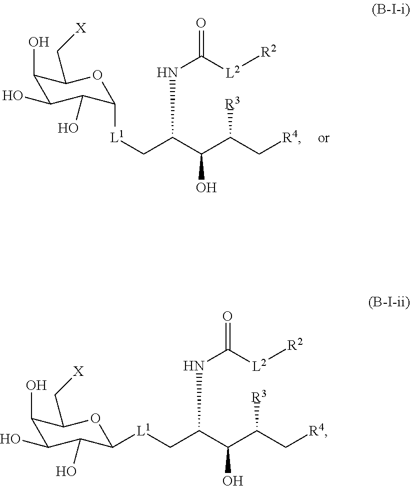

or a pharmaceutically acceptable salt thereof.

In some embodiments, the compound of Formula (B-I) is a compound of Formula (B-II):

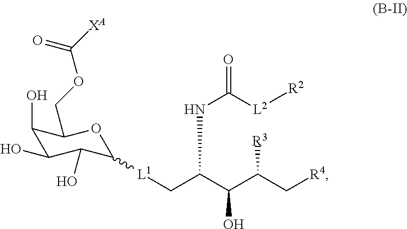

wherein XA is —R⁷ or —NR⁹R¹⁰, or a pharmaceutically acceptable salt thereof. In some embodiments, R⁷ is alkyl optionally substituted with one or more R^{A}; and each R^{A} is independently aryl. In some embodiments, R⁷ is alkyl substituted with one or more aryl. In some embodiments, R⁷ is ethyl substituted with one or more aryl. In some embodiments, R⁷ is

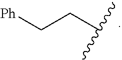 [SMILES: *CCc1ccccc1]

In some embodiments each of R⁹ and R¹⁰ is independently hydrogen or cycloalkyl. In some embodiments, one of R⁹ and R¹⁰ is hydrogen and the other of R⁹ and R¹⁰ cycloalkyl. In some embodiments, one of R⁹ and R¹⁰ is hydrogen and the other of R⁹ and R¹⁰ is

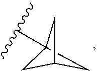 [SMILES: *C12CC(C1)C2]

optionally substituted with halo. In one variation, one of R⁹ and R¹⁰ is hydrogen and the other of R⁹ and R¹⁰ is

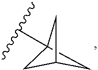 [SMILES: *C12CC(C1)C2]

optionally substituted with fluoro. In another variation, one of R⁹ and R¹⁰ is hydrogen and the other of R⁹ and R¹⁰ is

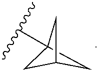 [SMILES: *C12CC(C1)C2]

In some embodiments, the compound of Formula (B-I) is a compound of Formula (B-III):

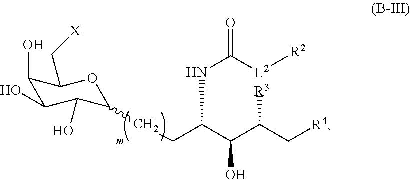

or a pharmaceutically acceptable salt thereof.

In one aspect, provided herein is a compound of Formula (B-IV):

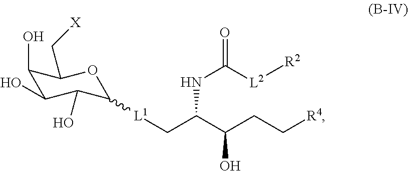

or a pharmaceutically acceptable salt thereof.

In some embodiments of a compound of Formula (B) or (B-I), or a pharmaceutically acceptable salt thereof, R² is cycloalkyl or heterocyclylalkyl, wherein the cycloalkyl or heterocyclylalkyl is optionally substituted with one or more R^{b}; and R^{b} is, for each occurrence, independently oxo or halo. In some embodiments, R² is heterocyclylalkyl. In some embodiments, R² is cycloalkyl. In some embodiments, R² is propellane. In some embodiments, R² is [1.1.1]-propellane. In some embodiments, R² is cycloalkyl or heterocyclylalkyl substituted with one or more R^{b}; and R^{b} is, for each occurrence, independently oxo or halo. In some embodiments, R² is

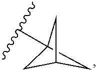 [SMILES: *C12CC(C1)C2]

optionally substituted with halo. In certain variations, R² is

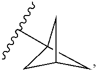 [SMILES: *C12CC(C1)C2]

optionally substituted with fluoro. In some variations, R² is

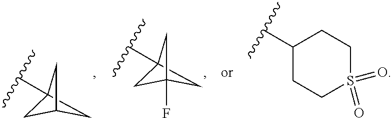 [SMILES: *C12CC(C1)C2 ; *C12CC(F)(C1)C2 ; *C1CCS(=O)(=O)CC1]

In some embodiments of a compound of Formula (B) or (B-I), such as a compound of formula (B-II), or (B-III), or a pharmaceutically acceptable salt thereof, R³ is hydrogen or —OH. In some embodiments, R³ is hydrogen. In some embodiments, R³ is —OH.

In some embodiments of a compound of Formula (B) or (B-I), such as a compound of formula (B-II), (B-III), or (B-IV), or a pharmaceutically acceptable salt thereof, R⁴ is alkyl. In some embodiments, R⁴ is C₈-C₁₅ alkyl. In some embodiments, R⁴ is C₁₃H₂₇.

In some embodiments of a compound of Formula (B) or (B-I), such as a compound of formula (B-II), or (B-IV), or a pharmaceutically acceptable salt thereof, L¹ is —O— or —(CH₂)ₘ—. In some embodiments, L¹ is —O—. In some embodiments, L¹ is —(CH₂)ₘ—; and m is 1 or 2. In some embodiments, L¹ is —(CH₂)ₘ—; and m is 1. In some embodiments, L¹ is —(CH₂)ₘ—; and m is 2.

In some embodiments of a compound of Formula (B) or (B-I), such as a compound of formula (B-II), (B-III), or (B-IV), or a pharmaceutically acceptable salt thereof, L² is alkylene. In some embodiments, L² is C₈-C₃₀ alkylene. In some embodiments, L² is —(CH₂) 23-.

In some embodiments of a compound of Formula (B) or (B-I), or a pharmaceutically acceptable salt thereof, X is —OH, —OC(O)R⁷, —OC(O)NR⁹R¹⁰, or —N(R^{a})C(O)R¹³, wherein, when X is OH or —N(R^{a})C(O)R¹³, either L¹ is —(CH₂)ₘ—or R³ is hydrogen.

In some embodiments of a compound of Formula (B) or (B-I), such as a compound of formula (B-III), or (B-IV), or a pharmaceutically acceptable salt thereof, X is —OH and L¹ is —(CH₂)ₘ—. In some embodiments, X is —OH and R³ is hydrogen.

In some embodiments of a compound of Formula (B) or (B-I), such as a compound of formula (B-III), or (B-IV), or a pharmaceutically acceptable salt thereof, X is —N(R^{a})C(O)R¹³ and L¹ is —(CH₂)ₘ—. In some embodiments, X is —N(R^{a})C(O)R¹³ and R³ is hydrogen. In some embodiments, X is —N(R^{a})C(O)R¹³; R^{a} is hydrogen or C₁₋₆ alkyl; and R¹³ is cycloalkyl. In some embodiments, X is —N(R^{a})C(O)R¹³; R^{a} is hydrogen; and R¹³ is cycloalkyl. In some embodiments, X is

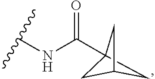 [SMILES: *NC(=O)C12CC(C1)C2]

wherein

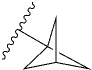 [SMILES: *C12CC(C1)C2]

is optionally substituted with halo. In other variations,

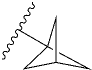 [SMILES: *C12CC(C1)C2]

is optionally substituted with fluoro. In other variations,

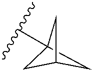 [SMILES: *C12CC(C1)C2]

is unsubstituted.

In some embodiments of a compound of Formula (B) or (B-I), such as a compound of formula (B-II), (B-III), or (B-IV), or a pharmaceutically acceptable salt thereof, X is —OC(O)R⁷; R⁷ is alkyl optionally substituted with one or more R^{A}; and each R^{A} is independently aryl. In some embodiments, X is —OC(O)R⁷; and R⁷ is alkyl substituted with one or more aryl. In some embodiments, X is —OC(O)R⁷; and R⁷ is ethyl substituted with one or more aryl. In some embodiments, X is

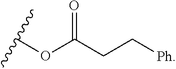 [SMILES: *OC(=O)CCc1ccccc1]

In some embodiments of a compound of Formula (B) or (B-I), such as a compound of formula (B-II), (B-III), or (B-IV), or a pharmaceutically acceptable salt thereof, X is —OC(O)NR⁹R¹⁰; and each of R⁹ and R¹⁰ is independently hydrogen or cycloalkyl. In some embodiments, X is —OC(O)NR⁹R¹⁰; one of R⁹ and R¹⁰ is hydrogen and the other of R⁹ and R¹⁰ cycloalkyl. In some embodiments, X is

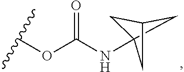 [SMILES: *OC(=O)NC12CC(C1)C2]

wherein

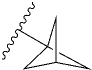 [SMILES: *C12CC(C1)C2]

is optionally substituted with halo. In other variations,

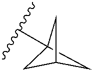 [SMILES: *C12CC(C1)C2]

is optionally substituted with fluoro. In other variations,

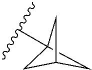 [SMILES: *C12CC(C1)C2]

is unsubstituted. In some embodiments of a compound of formula (I), such as a compound of formula (B), (B-I), (B-II), (B-III), or (B-IV), or a pharmaceutically acceptable salt thereof, the compound is selected from Table 1. In some embodiments, the compound is selected from Compounds I-1 to I-5 of Table 1. In some embodiments, the compound is selected from Compounds I-I to I-12 of Table 1. In some embodiments, exemplary compounds of Formulas (B), (B-I), (B-II), (B-III), and/or (B-IV) disclosed herein are illustrated in Table 1.

TABLE 1

| | |
|---|---|
| I-1 | 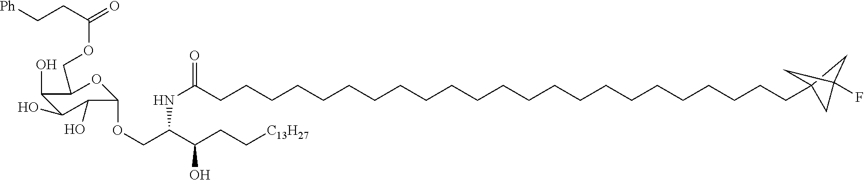 |
| | ((2R,3R,4S,5R,6S)-6-(((2S,3R)-2-(24-(3-fluorobicyclo[1.1.1]pentan-1- |
| | yl)tetracosanamido)-3-hydroxyoctadecyl)oxy)-3,4,5-trihydroxytetrahydro-2H- |
| | pyran-2-yl)methyl 3-phenylpropanoate |
| I-2 | 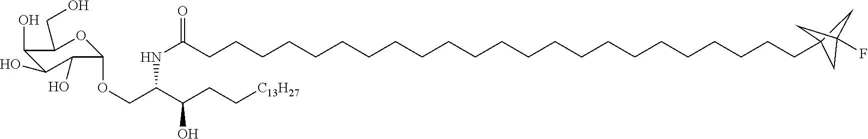 |
| | 24-(3-fluorobicyclo[1.1.1]pentan-1-yl)-N-((2S,3R)-3-hydroxy-1- |
| | (((2S,3R,4S,5R,6R)-3,4,5-trihydroxy-6-(hydroxymethyl)tetrahydro-2H-pyran-2- |
| | yl)oxy)octadecan-2-yl)tetracosanamide |
| I-3 | 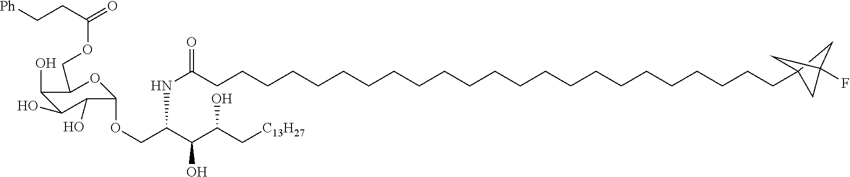 |
| | ((2R,3R,4S,5R,6S)-6-(((2S,3S,4R)-2-(24-(3-fluorobicyclo[1.1.1]pentan-1- |
| | yl)tetracosanamido)-3,4-dihydroxyoctadecyl)oxy)-3,4,5-trihydroxytetrahydro- |
| | 2H-pyran-2-yl)methyl 3-phenylpropanoate |
| I-4 | 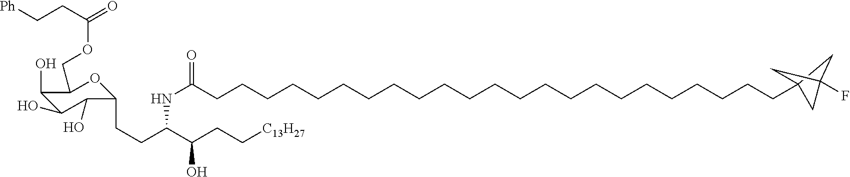 |
| | ((2R,3R,4R,5R,6R)-6-((3S,4R)-3-(24-(3-fluorobicyclo[1.1.1]pentan-1- |
| | yl)tetracosanamido)-4-hydroxynonadecyl)-3,4,5-trihydroxytetrahydro-2H-pyran- |
| | 2-yl)methyl 3-phenylpropanoate |
| I-5 | 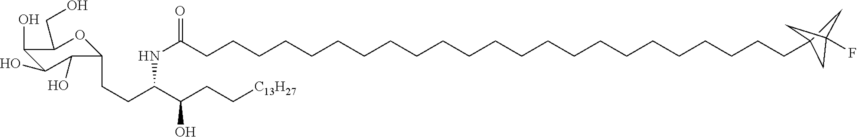 |
| | 24-(3-fluorobicyclo[1.1.1]pentan-1-yl)-N-((3S,4R)-4-hydroxy-1- |
| | ((2R,3R,4R,5R,6R)-3,4,5-trihydroxy- |
| | 6-(hydroxymethyl)tetrahydro-2H-pyran-2-yl)nonadecan-3-yl)tetracosanamide |
| I-6 | 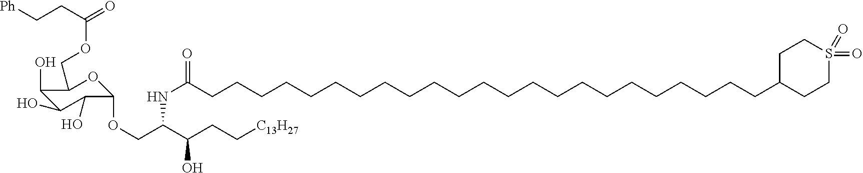 |
| | ((2R,3R,4S,5R,6S)-6-(((2S,3R)-2-(24-(1,1-dioxidotetrahydro-2H-thiopyran-4- |
| | yl)tetracosanamido)-3-hydroxyoctadecyl)oxy)-3,4,5-trihydroxytetrahydro-2H- |
| | pyran-2-yl)methyl 3-phenylpropanoate |
| I-7 | 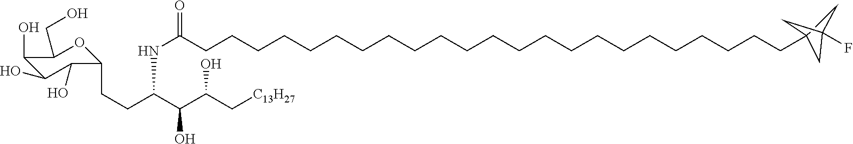 |
| | ((2R,3R,4R,5R,6R)-6-((3S,4R)-3-(24-(3-fluorobicyclo[1.1.1]pentan-1- |
| | yl)tetracosanamido)-4-hydroxynonadecyl)-3,4,5-trihydroxytetrahydro-2H-pyran- |
| | 2-yl)methyl 3-phenylpropanoate |
| I-8 | 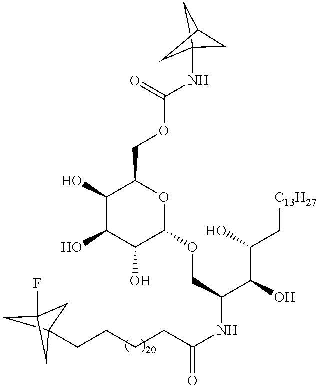 |
| | ((2R,3R,4S,5R,6S)-6-(((2S,3S,4R)-2-(24-(3-fluorobicyclo[1.1.1]pentan-1- |
| | yl)tetracosanamido)-3,4-dihydroxyoctadecyl)oxy)-3,4,5-trihydroxytetrahydro- |
| | 2H-pyran-2-yl)methylbicyclo[1.1.1]pentan-1-ylcarbamate |
| I-9 | 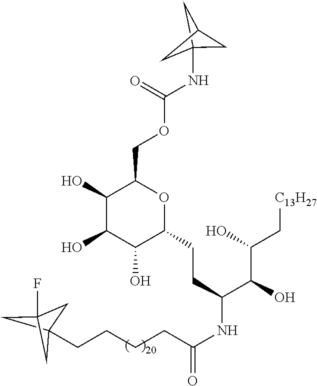 |
| | ((2R,3R,4R,5R,6R)-6-((3S,4S,5R)-3-(24-(3-fluorobicyclo[1.1.1]pentan-1- |
| | yl)tetracosanamido)-4,5-dihydroxynonadecyl)-3,4,5-trihydroxytetrahydro-2H- |
| | pyran-2-yl)methyl bicyclo[1.1.1]pentan-1-ylcarbamate |
| I-10 | 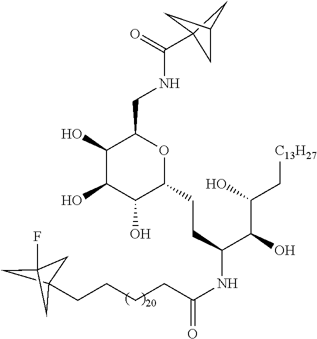 |
| | N-(((2R,3R,4R,5R,6R)-6-((3S,4S,5R)-3-(24-(3-fluorobicyclo[1.1.1]pentan-1- |
| | yl)tetracosanamido)-4,5-dihydroxynonadecyl)-3,4,5-trihydroxytetrahydro-2H- |
| | pyran-2-yl)methyl)bicyclo[1.1.1]pentane-1-carboxamide |
| I-11 | 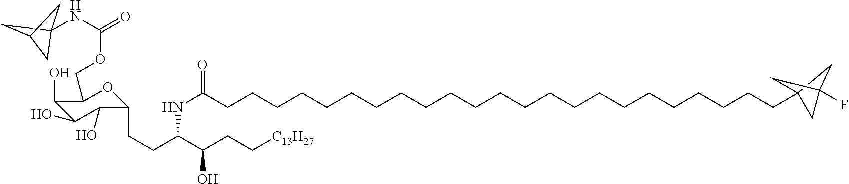 |
| | ((2R,3R,4R,5R,6R)-6-((3S,4R)-3-(24-(3-fluorobicyclo[1.1.1]pentan-1- |
| | yl)tetracosanamido)-4-hydroxynonadecyl)-3,4,5-trihydroxytetrahydro-2H-pyran- |
| | 2-yl)methyl bicyclo[1.1.1]pentan-1-ylcarbamate |
| I-12 | 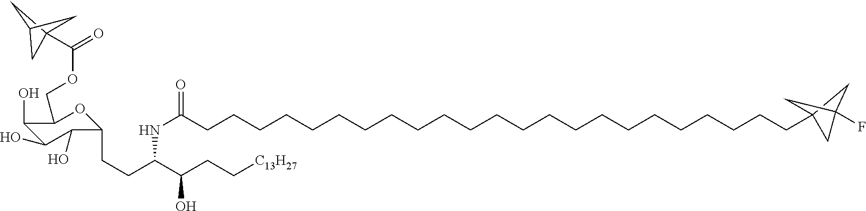 |
| | N-(((2R,3R,4R,5R,6R)-6-((3S,4R)-3-(24-(3-fluorobicyclo[1.1.1]pentan-1- |
| | yl)tetracosanamido)-4-hydroxynonadecyl)-3,4,5-trihydroxytetrahydro-2H-pyran- |
| | 2-yl)methyl)bicyclo[1.1.1]pentane-1-carboxamide |

In some embodiments of a compound of formula (I), such as a compound of formula (B), (B-I), (B-II), (B-III), or (B-IV), or a pharmaceutically acceptable salt thereof, the compound is selected from Table 1. In some embodiments, the compound is selected from Compounds II-1 to II-5 of Table 2. In some embodiments, the compound is selected from Compounds II-I to II-12 of Table 2. In some embodiments, exemplary compounds of Formulas (B), (B-I), (B-II), (B-III), and/or (B-IV) disclosed herein are illustrated in Table 2.

TABLE 2

| | |
|---|---|
| II-1 | 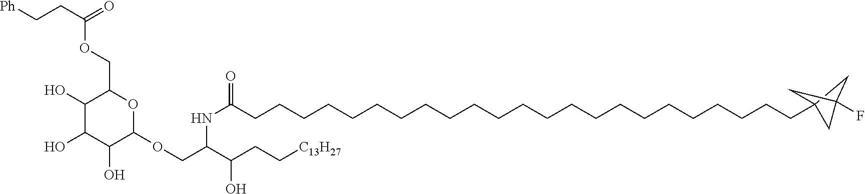 |
| | (6-((2-(24-(3-fluorobicyclo[1.1.1]pentan-1-yl)tetracosanamido)-3- |
| | hydroxyoctadecyl)oxy)-3,4,5-trihydroxytetrahydro-2H-pyran-2-yl)methyl 3- |
| | phenylpropanoate |
| II-2 | 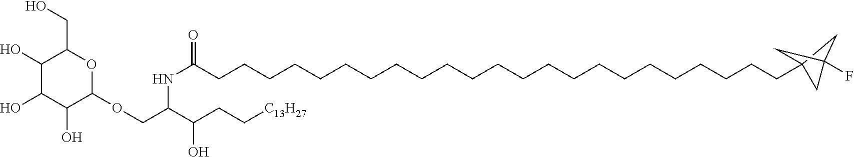 |
| | 24-(3-fluorobicyclo[1.1.1]pentan-1-yl)-N-(3-hydroxy-1-((3,4,5-trihydroxy-6- |
| | (hydroxymethyl)tetrahydro-2H-pyran-2-yl)oxy)octadecan-2-yl)tetracosanamide |
| II-3 | 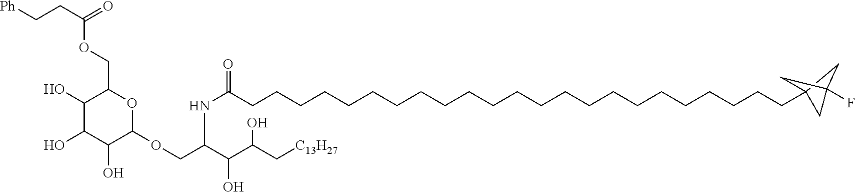 |
| | (6-((2-(24-(3-fluorobicyclo[1.1.1]pentan-1-yl)tetracosanamido)-3,4- |
| | dihydroxyoctadecyl)oxy)-3,4,5-trihydroxytetrahydro-2H-pyran-2-yl)methyl 3- |
| | phenylpropanoate |
| II-4 | 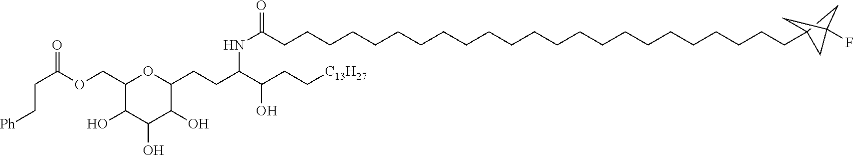 |
| | (6-(3-(24-(3-fluorobicyclo[1.1.1]pentan-1-yl)tetracosanamido)-4- |
| | hydroxynonadecyl)-3,4,5-trihydroxytetrahydro-2H-pyran-2-yl)methyl 3- |
| | phenylpropanoate |
| II-5 | 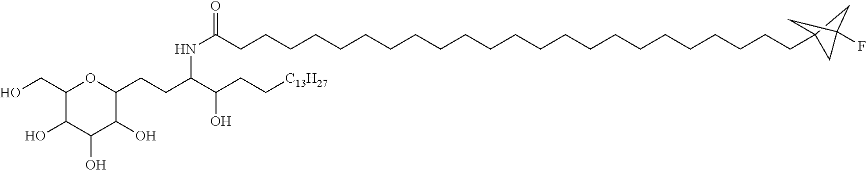 |
| | 24-(3-fluorobicyclo[1.1.1]pentan-1-yl)-N-(4-hydroxy-1-(3,4,5-trihydroxy-6- |
| | (hydroxymethyl)tetrahydro-2H-pyran-2-yl)nonadecan-3-yl)tetracosanamide |
| II-6 | 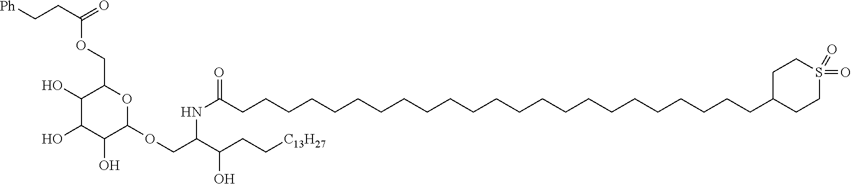 |
| | (6-((2-(24-(1,1-dioxidotetrahydro-2H-thiopyran-4-yl)tetracosanamido)-3- |
| | hydroxyoctadecyl)oxy)-3,4,5-trihydroxytetrahydro-2H-pyran-2-yl)methyl 3- |
| | phenylpropanoate |
| II-7 | 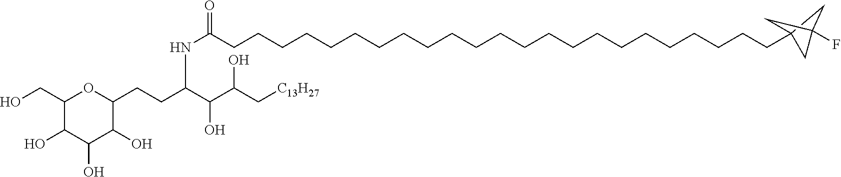 |
| | N-(4,5-dihydroxy-1-(3,4,5-trihydroxy-6-(hydroxymethyl)tetrahydro-2H-pyran- |
| | 2-yl)nonadecan-3-yl)-24-(3-fluorobicyclo[1.1.1]pentan-1-yl)tetracosanamide |
| I-8 | 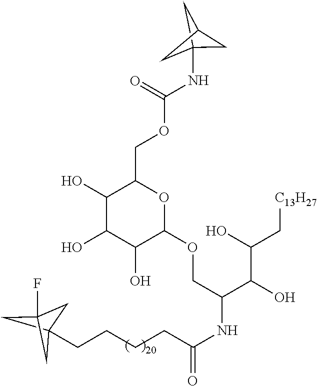 |
| | (6-((2-(24-(3-fluorobicyclo[1.1.1]pentan-1-yl)tetracosanamido)-3,4- |
| | dihydroxyoctadecyl)oxy)-3,4,5-trihydroxytetrahydro-2H-pyran-2-yl)methyl |
| | bicyclo[1.1.1]pentan-1-ylcarbamate |
| II-9 | 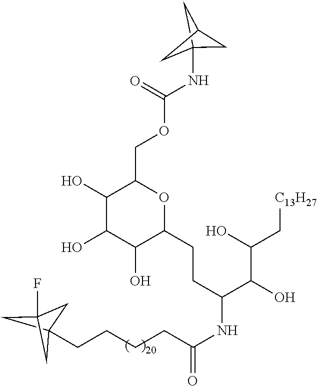 |
| | (6-(3-(24-(3-fluorobicyclo[1.1.1]pentan-1-yl)tetracosanamido)-4,5- |
| | dihydroxynonadecyl)-3,4,5-trihydroxytetrahydro-2H-pyran-2-yl)methyl |
| | bicyclo[1.1.1]pentan-1-ylcarbamate |
| II-10 | 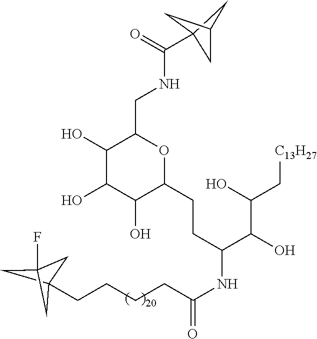 |
| | N-((6-(3-(24-(3-fluorobicyclo[1.1.1]pentan-1-yl)tetracosanamido)-4,5- |
| | dihydroxynonadecyl)-3,4,5-trihydroxytetrahydro-2H-pyran-2- |
| | yl)methyl)bicyclo[1.1.1]pentane-1-carboxamide |
| II-11 | 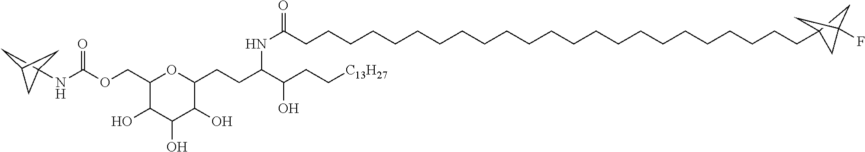 |
| | (6-(3-(24-(3-fluorobicyclo[1.1.1]pentan-1-yl)tetracosanamido)-4- |
| | hydroxynonadecyl)-3,4,5-trihydroxytetrahydro-2H-pyran-2-yl)methyl |
| | bicyclo[1.1.1]pentan-1-ylcarbamate |
| II-12 | 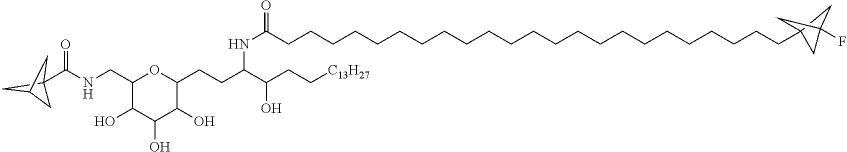 |
| | N-((6-(3-(24-(3-fluorobicyclo[1.1.1]pentan-1-yl)tetracosanamido)-4- |
| | hydroxynonadecyl)-3,4,5-trihydroxytetrahydro-2H-pyran-2- |
| | yl)methyl)bicyclo[1.1.1]pentane-1-carboxamide |

In one aspect, provided is a compound as set forth in Tables 1 and 2 above, or a pharmaceutically acceptable salt thereof. “Pharmaceutically acceptable salts” include, when appropriate, pharmaceutically acceptable base addition salts and acid addition salts. As used herein, the term “pharmaceutically acceptable” infers that the salt is not biologically or otherwise undesirable; for example, the material may be added to a pharmaceutical composition and administered to a subject without causing significant undesirable effects.

In another aspect, provided is a pharmaceutical composition, comprising any of the compounds described herein, including the compounds as set forth in Tables 1 and 2 above, or a pharmaceutically acceptable salt thereof; and at least one pharmaceutically acceptable carrier.

### III. Methods for Activating INKT Cells

As summarized above, aspects of the present disclosure also include methods for activating an iNKT cell. In embodiments, methods include contacting an iNKT cell with an amount of one or more of the compounds or a pharmaceutically acceptable salt thereof described herein sufficient to activate the iNKT cell. In some instances, a source of the iNKT cell is contacted in vitro. In other instances, a source of the iNKT cell is contacted in vivo (e.g., by administering to a subject as described in greater detail below). In still other instances, a source of the iNKT cell is contacted ex vivo.

In some embodiments provided herein, is a method for activating invariant natural killer T (INKT) cells in a subject in need thereof, comprising administering to the subject a compound of Formulas (B-I)-(B-IV), or a pharmaceutically composition comprising a compound of Formulas (B-I)-(B-IV) and at least one pharmaceutically acceptable carrier. In some embodiments, the method comprises contacting invariant natural killer T (iNKT) cells with a compound of Formulas (B-I)-(B-IV), or a pharmaceutically composition comprising a compound of Formulas (B-I)-(B-IV) and at least one pharmaceutically acceptable carrier. In some embodiments, contacting iNKT cells activates the iNKT cells. In some embodiments, the activated iNKT cells reduce the presence of senescent cells. In some embodiments, contacting the iNKT cells occurs in vitro. In some embodiments, contacting the iNKT cells occurs in vivo.

In some embodiments, methods include contacting one or more of the compounds described herein with iNKT cells in a manner sufficient to activate the iNKT cells, where the activated iNKT cells induce a TH1-type cytokine response (e.g., increase production of one or more cytokines selected from IFN-γ, IL-1B, IL-2, IL-3, IL-8, IL-12, IL-15, TNF-α, GM-CSF, RANTES, MIP-1α and MCP-1). In other instances, the activated iNKT cells induce a TH2-type cytokine response (e.g., increase production of one or more cytokines selected from IL-4, IL-6, IL-8, IL-10, IL-13, RANTES, MIP-1α and MCP-1). In some instances, activating the iNKT cells with one or more of the subject compounds is sufficient to increase cytokine production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase interleukin-2 (IL-2) production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase interleukin-4 (IL-4) production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase interleukin-6 (IL-6) production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase interferon gamma (IFNγ) production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase tumor necrosis factor (TNFα) production by 1% or more as compared to a suitable control (e.g., iNKT cells not contacted with the compound or a control compound), such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more.

In certain instances, activating the iNKT cells with one or more of the subject compounds is sufficient to increase cytokine production as compared to contacting the iNKT cells with a-galactosylceramide (α-GalCer), such as where cytokine production (e.g., increasing one or more of IFN-γ, IL-1B, IL-2, IL-3, IL-8, IL-12, IL-15, TNF-α, GM-CSF, RANTES, MIP-la and MCP-1 or IL-4, IL-6, IL-8, IL-10, IL-13, RANTES, MIP-1a and MCP-1) is higher by activating the iNKT cells with one or more of the subject compounds than when the iNKT cells are contacted with a-galactosylceramide.

In some embodiments, the compounds of the present disclosure increase cytokine production by 1% or more as compared to α-galactosylceramide, such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more as compared to α-galactosylceramide.

In some instances, the compound forms a complex with a CD1 molecule on an antigen-presenting cell. In certain instances, the CD1 molecule is a CD1d molecule. In some instances, the receptor on the T lymphocyte is a T cell receptor. In some instances, the compound stimulates at least one other lymphocyte to produce the cytokine response in some instances the at least one other lymphocyte is a T helper cell. In some embodiments, methods include activating iNKT cells with the subject compounds in a manner sufficient to modulate an immune response in a subject.

In practicing the subject methods, the iNKT cells may be contacted with the subject compounds for a duration of 1 minute or more, such as for 2 minutes or more, such as for 3 minutes or more, such as for 4 minutes or more, such as for 5 minutes or more, such as for 10 minutes or more, such as for 15 minutes or more, such as for 30 minutes or more, such as for 60 minutes or more, such as for 2 hours or more, such as for 6 hours or more, such as for 12 hours or more, such as for 18 hours or more and including for 24 hours or more. In certain embodiments, the production of one or more cytokines may be assessed (e.g., quantified) after contacting the compound with the iNKT cells. In some instances, the production of cytokines is assessed in real time (i.e., continuously monitored). In other instances, the production of cytokines is assessed at predetermined time intervals, such as every 1 minute, every 15 minutes, every 30 minutes, every 60 minutes, every 2 hours, every 4 hours, every 6 hours, every 12 hours, every 18 hours, including every 24 hours.

In some embodiments, contacting iNKT cells with one or more of the compounds of the present disclosure is sufficient to activate iNKT cells and to reduce the presence of or induce the killing of senescent cells. In certain embodiments, the senescent cells are senescent cells having an inflammatory secretome. For example, activating iNKT cells with the subject compounds according to these embodiments produces a cytotoxic effect against senescent cells. In some instances, methods include activating iNKT cells with the subject compounds in a manner sufficient to reduce the presence of or induce the killing of senescent cells in vitro. In some instances, methods include activating iNKT cells with the subject compounds in a manner sufficient to reduce the presence of or induce the killing of senescent cells in vivo (such as by administering the compound to a subject as part of a pharmaceutical composition described below). In some instances, activating iNKT cells with the subject compounds is sufficient to reduce the presence of senescent cells by 1% or more, such as by 2% or more, such as by 3% or more, such as by 4% or more, such as by 5% or more, such as by 10% or more, such as by 15% or more, such as by 20% or more, such as by 25% or more, such as by 50% or more, such as by 75% or more, such as by 90% or more, such as by 95% or more and including by 99% or more. In certain instances, the subject compounds eliminate the presence of senescent cells (e.g., where activation of iNKT cells reduces senescent present by 100%). The reduction in the presence of senescent cells may be assessed (e.g., quantified) after contacting the compound with the iNKT cells.

In some embodiments, iNKT cells activated by contact with the compounds described herein selectively reduce the presence of or selectively induce killing of senescent cells while maintaining (i.e., not killing) healthy cells. In some instances, contacting iNKT cells activated with the compounds of the present disclosure is sufficient to reduce the presence of senescent cells while maintaining 75% or more of the healthy cells, such as 80% or more, such as 85% or more, such as 90% or more, such as 95% or more, such as 97% or more, such as 99% or more, such as 99.9% or more and including 99.99% or more. In certain instances, iNKT cells activated by contact with compounds of the present disclosure selectively reduce the presence of senescent cells without any effect on healthy cells (i.e., 100% of healthy cells are maintained).

In some instances, the reduction of senescent cells may be assessed in real time (i.e., continuously monitored). In other instances, the reduction of senescent cells is assessed at predetermined time intervals, such as every 1 minute, every 15 minutes, every 30 minutes, every 60 minutes, every 2 hours, every 4 hours, every 6 hours, every 12 hours, every 18 hours, including every 24 hours.

Aspects of the present disclosure also include administering one or more of the compounds described herein, such as a compound of Formulas (A-I)-(A-IX), or Formulas (B) or (B-I)-(B-IV), including the exemplary compounds of Tables 1 and 2 above, to a subject in need thereof. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

In certain embodiments, the subject is diagnosed as having an autoimmune disease, a fibrotic disorder (lung, kidney, liver), an allergic disease or a metabolic syndrome.

In some embodiments, the presented disclosed compounds, such as a compound of Formulas (A-I)-(A-IX), or Formulas (B) or (B-I)-(B-IV), are administered to the subject to treat a disease, disorder or condition by activating invariant natural killer T (INKT) cells.

In particular embodiments, the disease, disorder or condition is type 1 diabetes, type 2 diabetes, diabetic nephropathy, diabetic kidney disease, diabetic bladder dysfunction, diabetic retinopathy, diabetic macular edema, fatty liver disease, such as non-alcoholic fatty liver disease/metabolic-associated fatty liver disease (NAFLD/MAFLD), non-alcoholic steatohepatitis/metabolic dysfunction-associated steatohepatitis (NASH/MASH), cancer, an eye disease, age related macular degeneration, heart disease, heart failure, atherosclerosis, hypertension, kidney disease, cardiorenal syndrome, pathogen infection, rheumatoid arthritis, ulcerative colitis, multiple sclerosis, familial hypercholesteremia, giant cell arteritis, idiopathic pulmonary fibrosis (IPF), systemic lupus erythematosus, cachexia, Werner syndrome, Fuchs' endothelial dystrophy, glaucoma, cataracts, posterior non-infectious uveitis, chronic obstructive pulmonary disease, systemic sclerosis, pulmonary arterial hypertension, lipodystrophy, sarcopenia, osteoporosis, Duchenne muscular dystrophy, myotonic dystrophy, alopecia, post myocardial infarction, vitiligo, postural orthostatic tachycardia syndrome (POTS), medium-chain acyl-coenzyme A dehydrogenase deficiency (MCAD), Sjögren's syndrome, scleroderma, Hashimoto Disease, ankylosing spondylitis, fibromyalgia, sarcoidosis, hepatitis, Raynaud's Syndrome, chronic inflammatory response syndrome or mold illness, celiac, Crohn's disease, inflammatory bowel disease, pemphigus, Stiff Person Syndrome (SPS), Primary Biliary Cholangitis (PBC) (cholestatic diseases), psoriatic arthritis, chronic inflammatory demyelinating polyradiculoneuropathy (CIDP), motor neuron disease, granulomatosis with polyangiitis (GPA) (Wegener's disease), amyotrophic lateral sclerosis (ALS), primary open angle glaucoma (POAG), myasthenia gravis, or presbyopia, or any combination thereof. In some embodiments, the subject is diagnosed (for example, by clinical laboratory test or by a qualified healthcare professional) as having or exhibiting at least one symptom of multiple sclerosis, articular rheumatism, psoriasis, Crohn's disease, leukoderma vulgaris, Behcet's disease, collagenosis, uveitis, Sjögren's syndrome, autoimmune cardiomyositis, autoimmune liver disease, autoimmune gastritis, pemphigus, Guillain-Barre syndrome, HTLV-1-related myelopathy or fulminant hepatitis.

In some embodiments, methods include administering one or more of the compounds to treat a subject for an infectious disease, such as one caused by a pathogenic microbe, including for example viruses, bacteria, fungi, protozoa and multicellular parasites. In one example, the infectious disease is by a virus selected from Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arena viridae, Reoviridae, Birnaviridae, Hepadnaviridae, Parvoviridae, Papovaviridae, Adenoviridae, Herpesviridae, Poxyiridae and Iridoviridae. In another example, the infectious disease is caused by a bacteria selected from Helicobacter pylori, Borellia burgdorferi, Legionella pneumophilia, Klebsiella Pneumoniae, Mycobacteria sps, Staphylococcus aureus, Neisseria gonorrhoeae, Neisseria meningitidis, Listeria monocytogenes, Streptococcus pyogenes, Streptococcus agalactiae, Streptococcus, Streptococcus faecalis, Streptococcus bovis, Streptococcus pneumoniae, pathogenic Campylobacter sp., Enterococcus sp., Chlamidia sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Actinomyces israelli, Sphingomonas capsulata and Francisella tularensis.

Compounds as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

Depending on the condition being treated, the amount of compound administered to the subject may vary, such as ranging from about 100 mg/day to about 10,000 mg/day, such as from about 10 mg/day to about 9000 mg/day, such as from 50 mg/day to about 8000 mg/day, such as from about 100 mg/day to about 7000 mg/day, such as from about 500 mg/day to about 6000 mg/day, including from about 600 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg.

In certain embodiments, protocols may include multiple dosage intervals. By “multiple dosage intervals” is meant that two or more dosages of the compound is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

The cycles of drug administration may be repeated for 1, 2, 3, 4, 5, 6, 7, 8 or more than 8 dosage cycles, for a total period of 6 months or 1 year or 2 years or 3 years or 4 years or more. In certain embodiments, one or more of the subject compounds are administered for the rest of the subject's lifetime.

In certain embodiments, compounds of the present disclosure can be administered prior to, concurrent with, or subsequent to other therapeutic agents for treating the same or an unrelated condition. If provided at the same time as another therapeutic agent, the present compounds may be administered in the same or in a different composition. Thus, the compounds of interest and other therapeutic agents can be administered to the subject by way of concurrent therapy. By “concurrent therapy” is intended administration to a subject such that the therapeutic effect of the combination of the substances is caused in the subject undergoing therapy. For example, concurrent therapy may be achieved by administering the compounds of the present disclosure with a pharmaceutical composition having at least one other agent, such as an anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Where a compound of the present disclosure is administered concurrently with a second therapeutic agent to treat the same condition (e.g., a chemotherapeutic, an anti-viral drug, etc.) the weight ratio of the subject compound to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the subject compound to second therapeutic agent may range between 1:1 and 1:5; 1:5 and 1:10; 1:10 and 1:15; or 1:15 and 1:25. Alternatively, the weight ratio of the second therapeutic agent to the subject compound ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the second therapeutic agent the subject compound may range between 1:1 and 5:1; 5:1 and 10:1; 10:1 and 15:1; or 15:1 and 25:1.

Aspects of the present disclosure also include compositions having a pharmaceutically acceptable carrier and one or more of the compounds described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The compounds may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In certain embodiments, compositions of interest include liposomal or micellar compositions where the compounds described herein are liposome-based formulations or micelle-based formulations. The liposome-based formulation or micelle-based formulation of the subject compounds may be prepared by any convenient liposome or micelle forming protocol, such as for example by mechanical dispersion, solvent dispersion, or a detergent removal method. In certain instances, liposomes are formed by mechanical dispersion including by sonication, French pressure cell extrusion, freeze-thawing, lipid film hydration (e.g., by hand —shaking, mechanical agitation or freeze drying), micro-emulsification, membrane extrusion or using dried reconstituted vesicles. In certain embodiments, liposome-based formulations of the compounds described herein are prepared by thin-film rehydration followed by extrusion (e.g., through a filter of 5 nm or more, such as 10 nm or more, such as 25 nm or more, such as 50 nm or more, such as 100 nm or more, such as 150 nm or more, such as 200 nm or more, such as 250 nm or more, such as 300 nm or more and including extrusion through a filer of 500 nm or more).

In some embodiments, the liposome-based formulation or micelle-based formulation may be formed from a non-polymeric carrier material including but not limited to: sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monoolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridecanoate, glyceryl trimyristate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphoethanolamine, phosphoethanolamine-PEG (2000), phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols; and combinations and mixtures thereof. In certain embodiments, liposome-formulated compounds include phosphatidylcholine and cholesterol.

Each component used to prepare the liposome formulation or micelle formulation may vary as desired and may be present in an amount of 0.001 wt % or more for the liposome or micelle formulation, such as 0.005 wt % or more, such as 0.010 wt % or more, such as 0.05 wt % or more, such as 0.1 wt % or more, such as 0.5 wt % or more, such as 1 wt % or more, such as 2 wt % or more, such as 3 wt % or more, such as 4 wt % or more and including where each component is present an in amount of 5 wt % or more. Where more than one component is present (e.g., a phospholipid such as phosphatidylcholine and cholesterol), the ratio of the components may range from 0.001:1 to 1:0.001, such as from 0.005:1 to 1:0.005, such as from 0.01:1 to 1:0.01, such as from 0.05:1 to 1:0.05, such as from 0.1:1 to 1:0.1, such as from 0.5:1 to 1:0.5, such as from 0.6:1 to 1:0.6, such as from 0.7:1 to 1:0.7, such as from 0.8:1 to 1:0.8, such as from 0.9:1 to 1:0.9 and including where the ratio of the components is 1:1 (e.g., phosphatidylcholine to cholesterol ratio of 1:1). In one example, the liposome or micelle formulation may include a phospholipid component (e.g., phosphatidylcholine) and cholesterol, such as in a ratio that ranges from 0.001:1 to 1:0.001, such as from 0.005:1 to 1:0.005, such as from 0.01:1 to 1:0.01, such as from 0.05:1 to 1:0.05, such as from 0.1:1 to 1:0.1, such as from 0.5:1 to 1:0.5, such as from 0.6:1 to 1:0.6, such as from 0.7:1 to 1:0.7, such as from 0.8:1 to 1:0.8, such as from 0.9:1 to 1:0.9 and including where the ratio of the components is 1:1. In certain instances, the ratio of phospholipid component to cholesterol is about 2:1.

The compounds described herein, for example a compound according to Formula (A-I), (A-II), (A-III), (A-IV), (A-V), (A-VI), (A-VII), (A-VIII), (A-IX), (B), (B-I), (B-II), (B-III), and/or (B-IV), may be present in the liposome or micelle formulation in an amount of 0.001 wt % or more of the formulation, such as 0.005 wt % or more, such as 0.010 wt % or more, such as 0.05 wt % or more, such as 0.1 wt % or more, such as 0.5 wt % or more, such as 1 wt % or more, such as 2 wt % or more, such as 3 wt % or more, such as 4 wt % or more and including where the active agent compound is present an in amount of 5 wt % or more. The ratio of active agent compound (e.g., compound for activating invariant natural killer T cells such as a compound of Formula (A-I), (A-II), (A-III), (A-IV), (A-V), (A-VI), (A-VII), (A-VIII), (A-IX), (B), (B-I), (B-II), (B-III), and/or (B-IV), to each liposome component may range from 0.001:1 to 1:0.001, such as from 0.005:1 to 1:0.005, such as from 0.01:1 to 1:0.01, such as from 0.05:1 to 1:0.05, such as from 0.1:1 to 1:0.1, such as from 0.5:1 to 1:0.5, such as from 0.6:1 to 1:0.6, such as from 0.7:1 to 1:0.7, such as from 0.8:1 to 1:0.8, such as from 0.9:1 to 1:0.9 and including a ratio of the components is 1:1. In certain instances, the ratio is from 1:0.15 or 2:0.15. For example, a composition may include phosphatidylcholine, cholesterol and an active agent compound at a ratio of 2:1:0.15.

In certain embodiments, liposome formulations or micelle formulations may include an organic solvent, such for example one or more organic solvents selected from sterols such as cholesterol, stigmasterol, β-sitosterol, and estradiol; cholesteryl esters such as cholesteryl stearate; C₁₂₋₂₄ fatty acids such as lauric acid, myristic acid, palmitic acid, stearic acid, arachidic acid, behenic acid, and lignoceric acid; C₁₈₋₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl trimyristate, glyceryl tridecanoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₈₋₃₆ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphoethanolamine, phosphoethanolamine-PEG (2000), phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols; and combinations and mixtures thereof.

In pharmaceutical dosage forms, the compounds may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a single dose of the compound is administered. In other embodiments, multiple doses of the compound are administered. Where multiple doses are administered over a period of time, the compound may be administered, e.g., twice daily (qid), daily (qd), every other day (qod), every third day, three times per week (tiw), or twice per week (biw) over a period of time. For example, the compound may be administered qid, qd, qod, tiw, or biw over a period of from one day to about 2 years or more. For example, the compound may be administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including topical, intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units can comprise components in any relative amounts. For example, dose units can be from about 0.1% to 99% by weight of active ingredients (i.e., compounds described herein) per total weight of dose unit. In some embodiments, dose units can be from 10% to 50%, from 20% to 40%, or about 30% by weight of active ingredients per total weight dose unit.

### IV. Synthesis of INKT Activators

Compounds disclosed herein, including iNKT activator compounds having Formulas (A-I)-(A-IX) or Formulas (B), or (B-I)-(B-IV) can be prepared according to the following representative synthetic schemes, which can be adapted to prepare additional compounds according to Formulas (A-I)-(A-IX) or Formulas (B), or (B-I)-(B-IV), as is known to those of ordinary skill in the art of organic synthesis.

For example, Scheme 1 illustrates an exemplary method for making compound I-1 and analogs thereof.

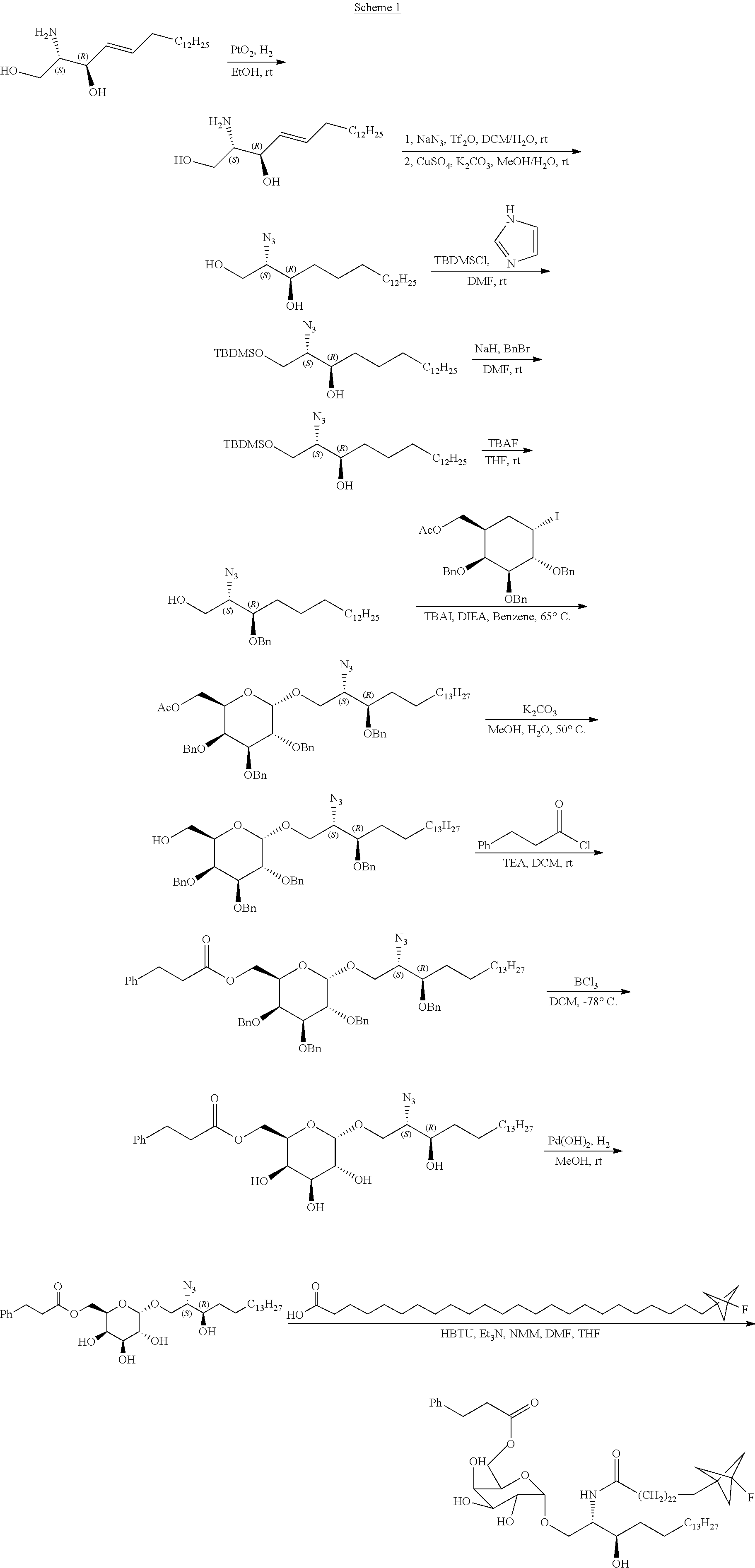

Scheme 2 illustrates an exemplary method for making compound I-4 and analogs thereof.

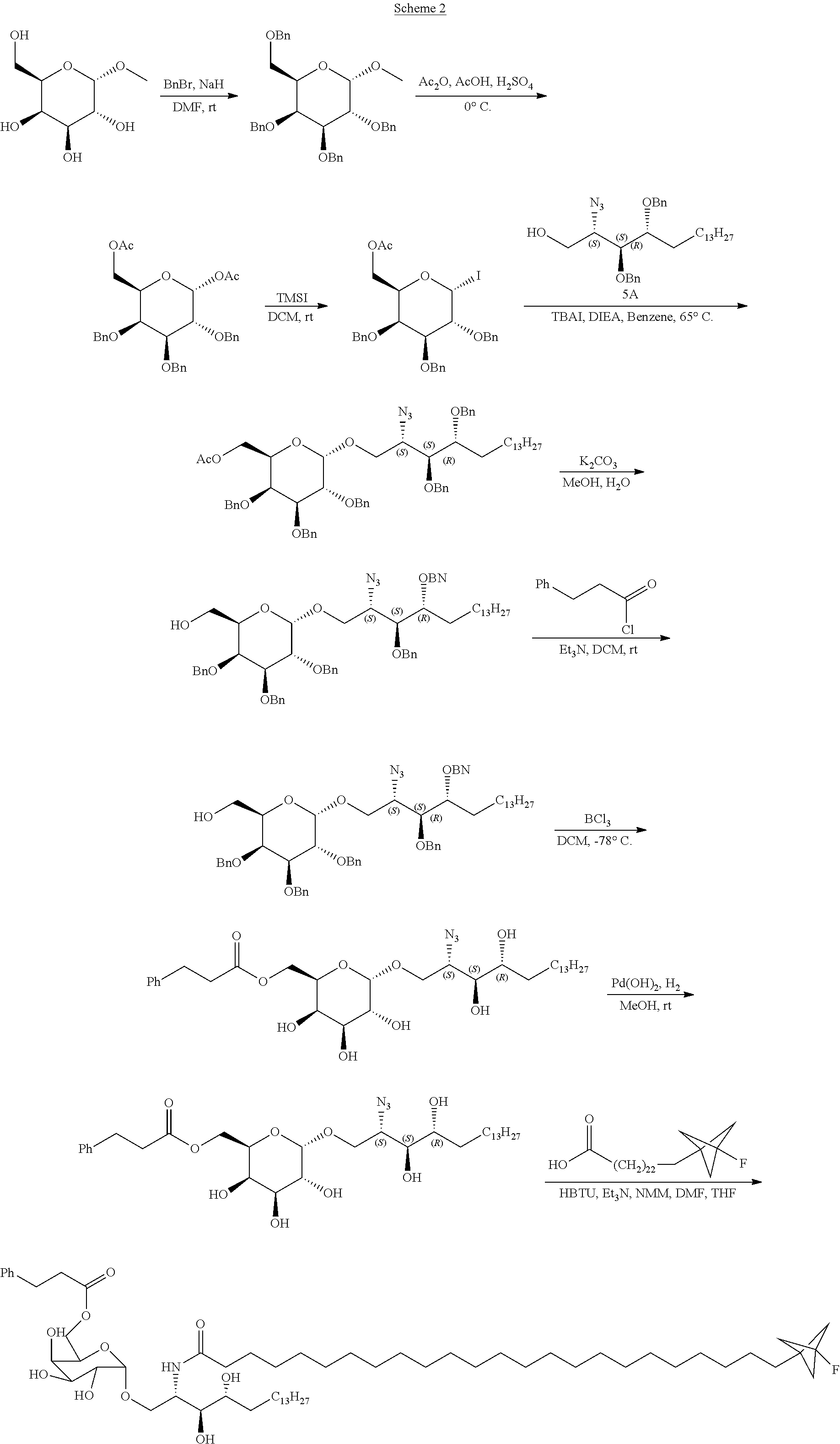

Scheme 3 provides an exemplary synthesis of a compound wherein, with reference to Formula (A-I), G is a five-membered heterocyclyl, such as a 5-membered heteroaryl.

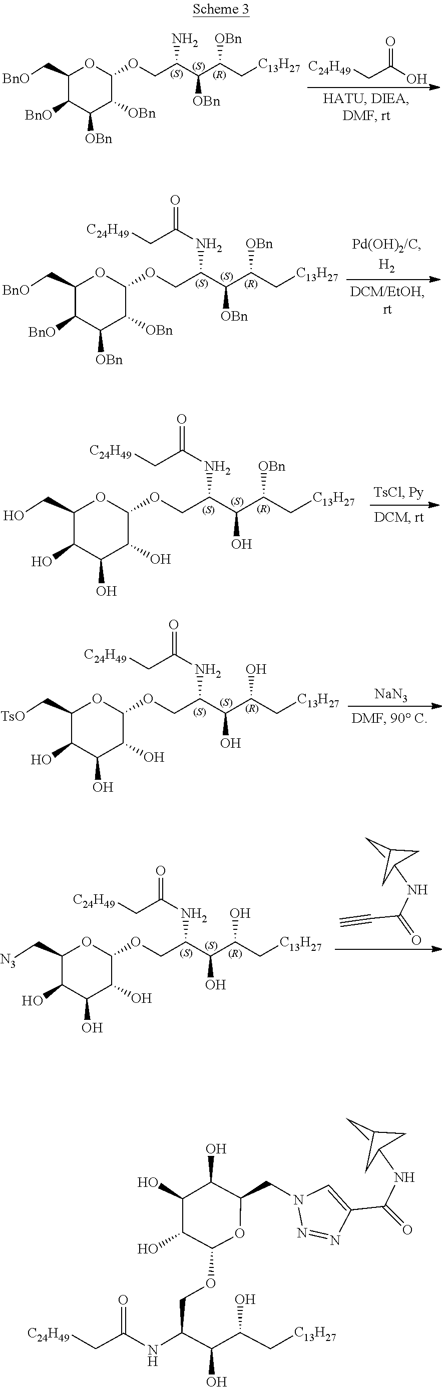

Scheme 4 is an exemplary synthesis of a versatile intermediate for the preparation of C-glycosides. With reference to Formulas (A-I)-(A-IX) or Formulas (B-I)-(B-IV) described herein, such C-glycosides are those wherein L¹ is —(CH₂). —or —(CH₂)ₘ—O-(m is 1 or 2).

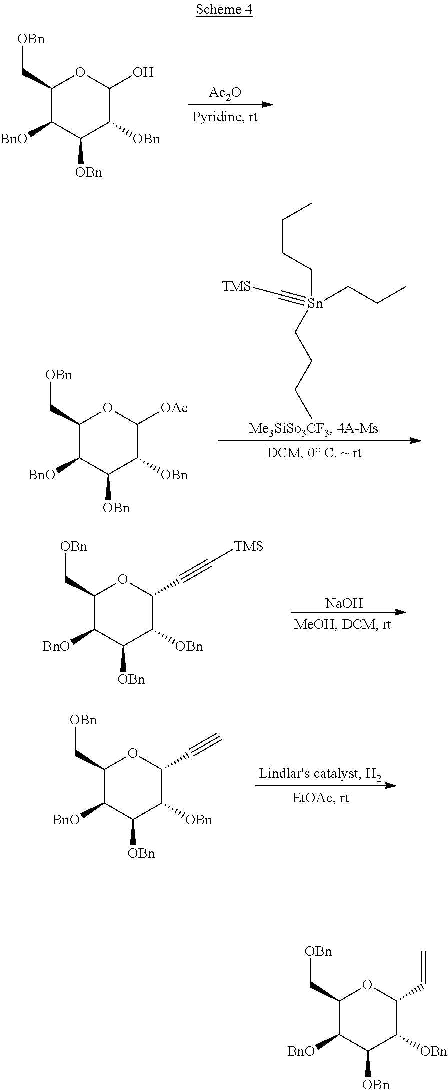

As will be readily apparent to those of skill in the art upon consideration of the present disclosure, the vinyl glycoside prepared according to Scheme 4 can be further elaborated to give compounds of Formulas (A-I)-(A-IX), or Formulas (B-I)-(B-IV), by a variety of techniques, such as cross-metathesis with another alkene-containing molecule. Scheme 5 illustrates one such route to C-glycosides of Formulas (A-I)-(A-IX), or Formulas (B-I)-(B-IV), employing the product of Scheme 4 as an intermediate.

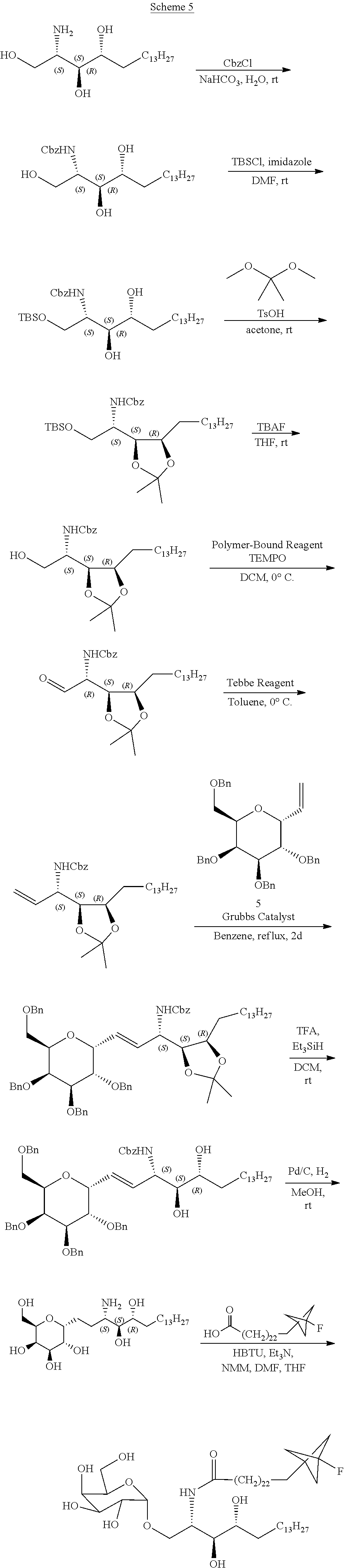

An analogous synthesis to that set forth in Scheme 5 is illustrated in Scheme 6. By using different starting materials as illustrated in Scheme 6, the exemplary method of Scheme 5 is adapted as is known to those of skill in the art to yield compound I-4.

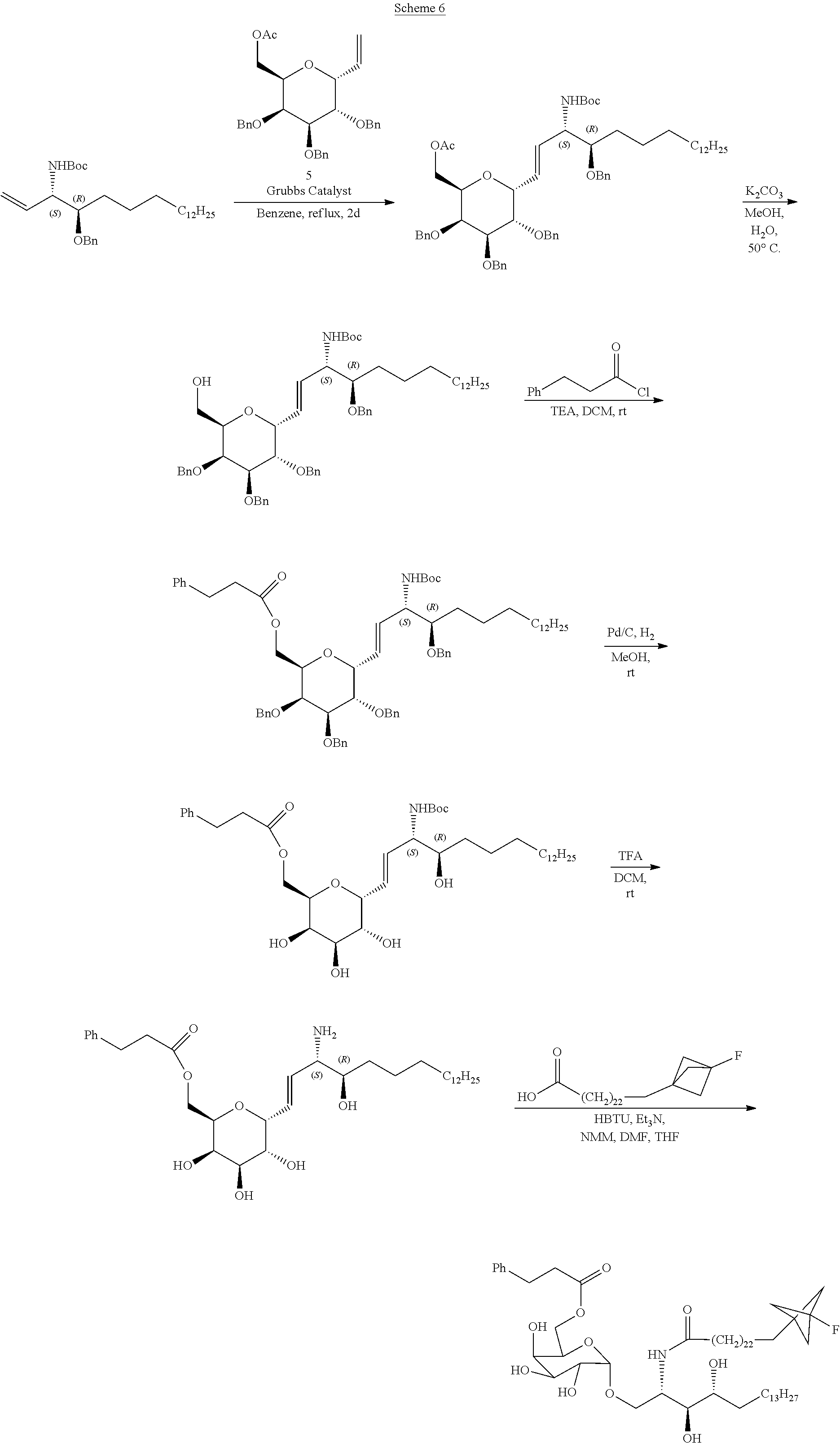

### ENUMERATED EMBODIMENTS

The following enumerated embodiments are representative of some aspects of the invention.
1. A compound of the formula:

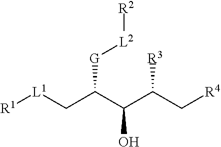

wherein:
R¹ is selected from

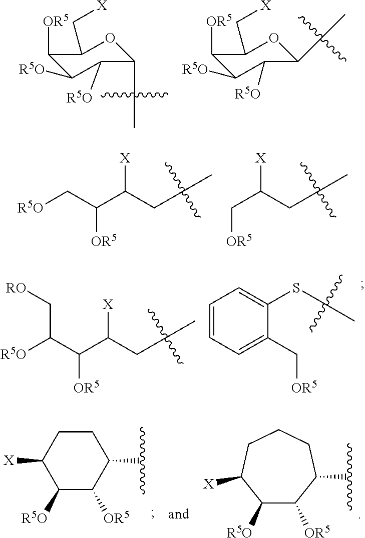

R² is selected from —Si(R^{a})₄, cycloalkyl and heterocyclylalkyl, wherein the cycloalkyl and heterocyclylalkyl groups are optionally substituted with one or more R^{a}, R^{b}, R^{d} substituted with one or more of the same or different R^{b}, and —OR^{a} substituted with one or more of the same or different R^{b};
R³ is hydrogen or —OR^{a};
R⁴ is C₈-C₁₅ alkyl;
R⁵ is selected from hydrogen, C₁₋₆ alkyl, —C(O)OR^{a}, —C(O)R^{a}, —C(O)NR^{c}R^{c};
X is selected from —OH, —OC(O)OR⁶, —OC(O)R⁷, —OC(O)OR⁸, —OC(O)NR⁹R¹⁰; —SR¹¹, —S(O)₂R¹²; —NR⁹R¹⁰; —NC(O)R¹³, —NC(O)OR¹⁴, —NC(O)NR⁹R¹⁰, 5-membered heterocyclyl, and 5-membered heteroaryl;
L¹ is —O—; —(CH₂)ₘ—; —S—, —S(O)₂—; —N(H)—; —O—(CH₂)ₘ—; or —(CH₂)ₘ—O—;
G is selected from —NR^{a}C(O)—; N(R^{a})C(R^{c})₂—; —C═C(R^{b})—; —NS(O) (R^{a})—; 5-membered heterocyclylalkyl, and 5-membered heteroaryl;
L² is a C₈-C₃₀ alkylene or alkenylene chain, optionally having one or more methylene replaced with a group selected from —O—, —S—, —S(O)₂—, —N(H)—, —N(R^{L})—, and Si(R^{c})₂—; and optionally having two or more adjacent methylene groups linked by a cyclopropyl moiety;
R^{a} is, for each occurrence, independently selected from hydrogen and C₁₋₆ alkyl;
R^{b} is, for each occurrence, independently selected from halogen and —CF₃,
R^{c} is, for each occurrence, hydrogen, alkyl, haloalkyl, or two R^{e} together with the atom to which they are attached form a heterocyclylalkyl ring that is unsubstituted or substituted with one or more halogen, C₁₋₆ alkyl, C(O)OR^{a}, —C(O)R^{a};
R^{d} is, for each occurrence, acyl, alkyl, heteroalkyl, cycloalkyl, heterocyclylalkyl, aryl, heteroaryl, each optionally substituted with one or more R^{B};
R^{L} is, for each occurrence, selected from C₁₋₁₅ alkyl, alkyl, C₃-8 cycloalkyl and C₂₋₇ heterocyclylalkyl;
m is, for each occurrence, 1 or 2;
each of R⁶, R⁷, R⁸, R⁹, R¹¹, R¹², R¹³, and R¹⁴ is independently hydrogen, alkyl, alkenyl, haloalkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A},
each of R⁹ and R¹⁰ is independently alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{A}, or R⁹ and R¹⁰ together with the atom to which they are attached form a heterocyclylalkyl ring or a heteroaryl ring that is unsubstituted or substituted with one or more R^{A};
each R^{A} is independently alkyl, heteroalkyl, cycloalkyl, heterocyclylalkyl, aryl, heteroaryl, oxo, —OR¹⁵, —N(R¹⁶)R¹⁷, —C(O)OR¹⁵, —N(R^{a})C(O)OR¹⁵, —N(R^{a})C(O)R¹⁵, —C(O)R¹⁵, —OC(O)R¹⁵, —OC(O)OR¹⁵, —C(O)N(R¹⁶)R¹⁷, —OC(O)N(R¹⁶)R¹⁷, wherein alkyl, heteroalkyl, cycloalkyl, heterocyclylalkyl, aryl, or heteroaryl is unsubstituted or substituted with alkyl, halogen, oxo, —OR^{a}, —NR(R¹⁶)R¹⁷, —C(O)R¹⁵, —OC(O)R¹⁵, —OC(O)OR¹⁵, or —OC(O)N(R¹⁶)R¹⁷;
each R¹⁵ is independently alkyl, heteroalkyl, cycloalkyl, or hydrogen, wherein alkyl, heteroalkyl, cycloalkyl is unsubstituted or substituted with one or more R^{B};
each of R¹⁶ and R¹⁷ is independently alkyl, or hydrogen, wherein alkyl, is unsubstituted or substituted with one or more R^{B}; or R¹⁶ and R¹⁷ together with the atom to which they are attached form a heterocyclylalkyl ring or heteroaryl ring, each of which is unsubstituted or substituted with one or more R^{B};
each R^{B} is independently halogen, amino, cyano, hydroxyl, alkyl, cycloalkyl, acyl wherein each cycloalkyl and acyl is unsubstituted or substituted with one or more halogen, amino, cyano, hydroxyl, alkyl, acetyl, or benzoyl;
provided that, when X is OH, L¹ is —(CH₂)ₘ—; —S—, —S(O)₂—; —N(H)—; —O—(CH₂)ₘ—; or —(CH₂)ₘ—O—.
2. The compound of embodiment 1, wherein R¹ is

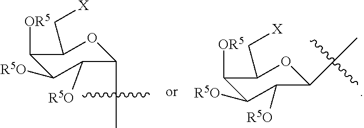

3. The compound of embodiment 1 or embodiment 2, wherein X is selected from

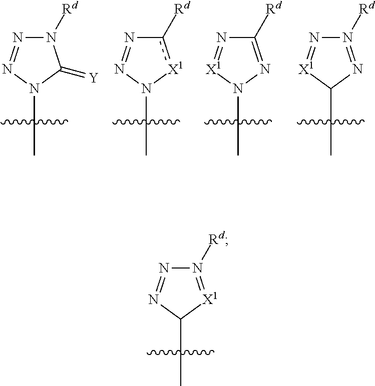

Y is O or S; and
X¹ is selected from —N—, —CH—, —C(R^{d})— and —C(R⁴)₂—.
4. The compound of any one of embodiments 1-3, wherein L¹ is selected from —(CH₂)ₘ—; —S—, —S(O)₂—; —N(H)—; —O—(CH₂)ₘ—; or —(CH₂)ₘ—O—.
5. The compound of any one of embodiments 1-3, wherein L¹ is —O—.
6. The compound of any one of embodiments 1-3, wherein G is selected from

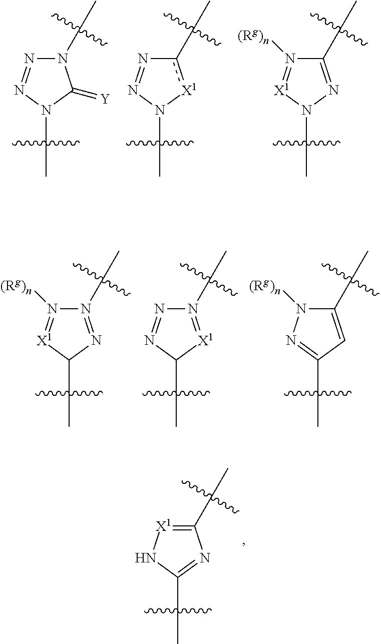

wherein
Y is O or S;
X¹ is selected from —N—, —CH—, —C(R⁴)— and —C(R⁴)₂—;
n is 0 or 1; and
R⁸ is hydrogen, C₁₋₆ alkyl, —C(O)OR^{a}, —C(O)R^{a}, or —C(O)NR^{c}R^{c}.
7. The compound of any one of embodiments 1-3, wherein G is selected from

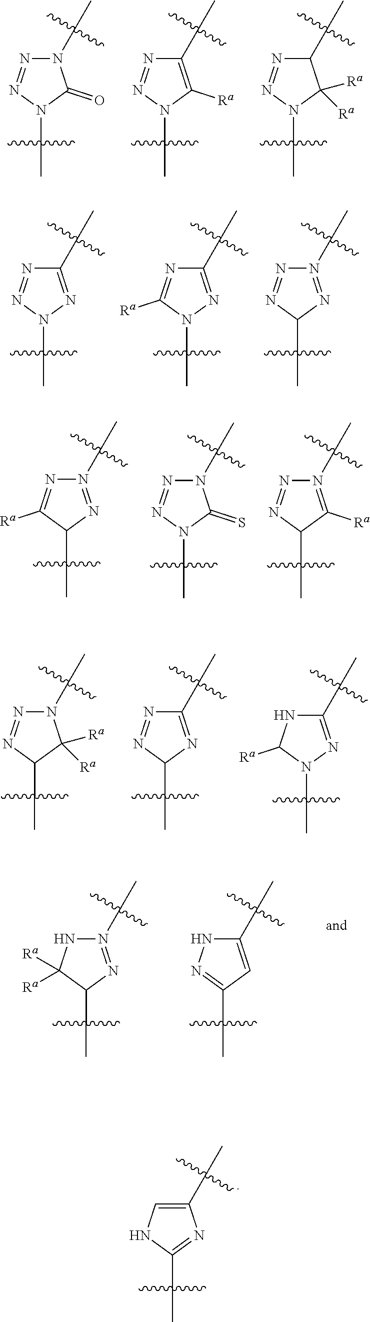

8. The compound of any one of embodiments 1-3, wherein G is selected from —NR^{a}C(O)—; —N(R^{a})C(R^{c})₂—; —C═C(R^{b})—; —NS(O) (R^{a})—.
9. The compound of any one of embodiments 1-3, wherein G is —C═C(R^{b})—, and R^{b} is fluoro.
10. The compound of one of embodiments 1-3, wherein G is —N(R^{a})C(R^{c})₂—.
11. The compound of embodiment 10, wherein one R^{e} is trifluoromethyl and the other is hydrogen.
12. The compound of embodiment 10, wherein two R^{c} together with the carbon atom to which they are attached form a heterocyclylalkyl ring.
13. The compound of embodiment 12, wherein the heterocyclylalkyl ring is an oxetane ring.
14. The compound of any one of embodiments 1-13, wherein L² is unsaturated.
15. The compound of any one of embodiments 1-13, wherein L¹ comprises at least one cyclopropanyl.
16. The compound of embodiment 1, wherein R² is selected from R² is selected from

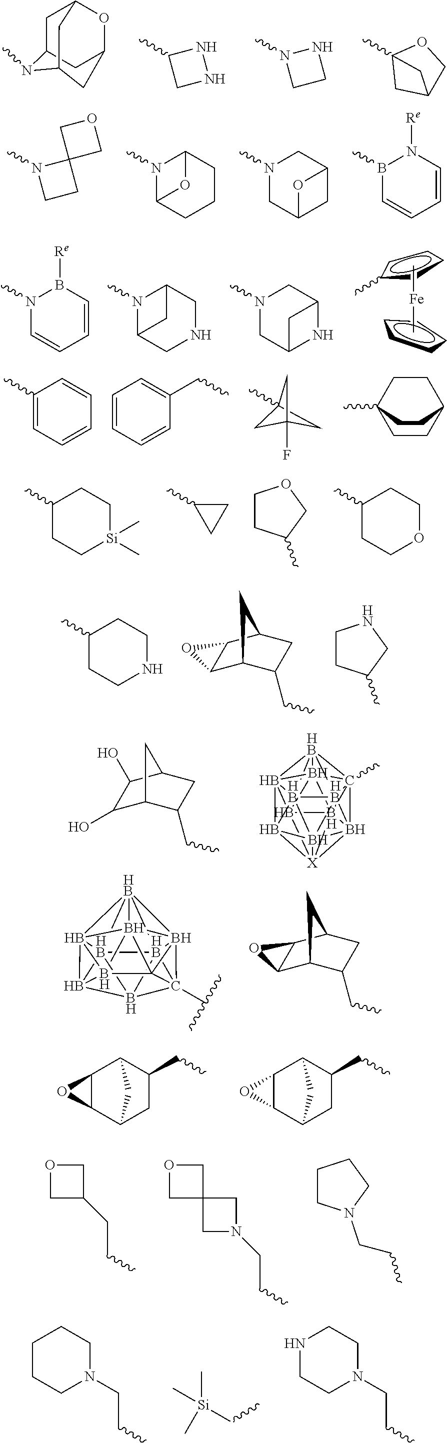

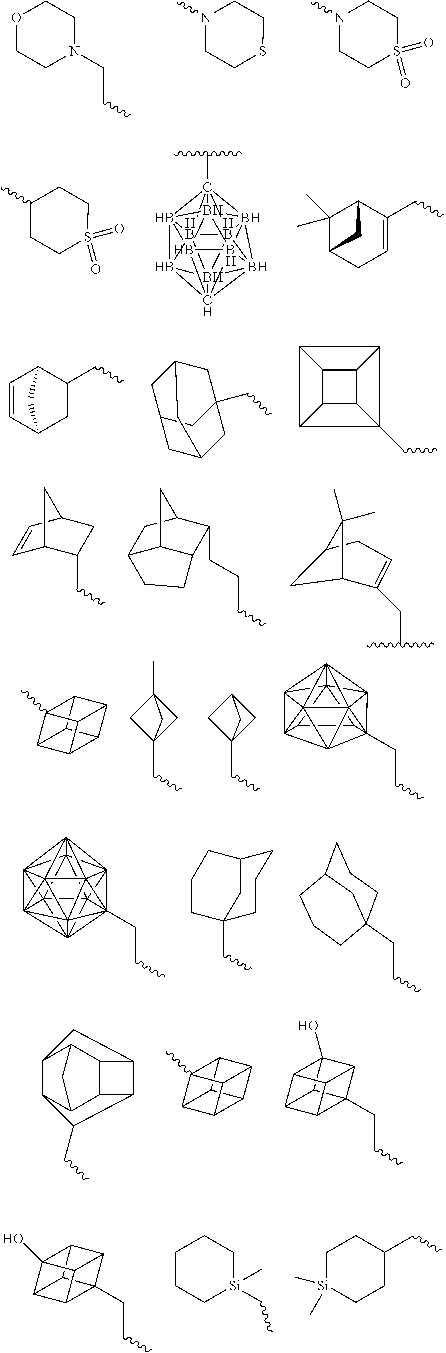

each optionally substituted with one or more R^{e}, wherein R^{e} is, for each occurrence, selected from halogen, C₁₋₁₅ alkyl, C₁₋₆ haloalkyl, —C(O)OR^{a}, —C(O)R^{a} and —C(O)NR^{c}R^{c}.
17. The compound of any one of embodiments 1-16, wherein R³ is hydrogen.
18. The compound of any one of embodiments 1-16, wherein R³ is —OH.
19. The compound of any one of embodiments 1-18, wherein R⁴ is C₁₃H₂₇.
20. The compound of any one of embodiments 1-19, wherein R⁵ is hydrogen.
21. A compound selected from the group consisting of

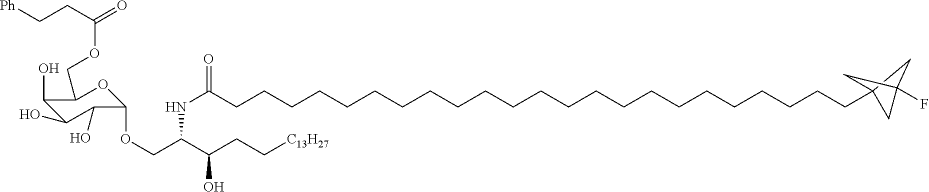

((2R,3R,4S,5R,6S)-6-(((2S,3R)-2-(24-(3-fluorobicyclo[1.1.1]pentan-1-yl)tetracosanamido)-3-hydroxyoctadecyl)oxy)-3,4,5-trihydroxytetrahydro-2H-pyran-2-yl)methyl 3-phenylpropanoate

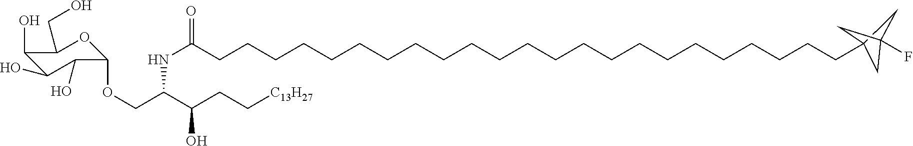

24-(3-fluorobicyclo[1.1.1]pentan-1-yl)-N-((2S,3R)-3-hydroxy-1-(((2S,3R,4S,5R,6R)-3,4,5-trihydroxy-6-(hydroxymethyl)tetrahydro-2H-pyran-2-yl)oxy) octadecan-2-yl)tetracosanamide

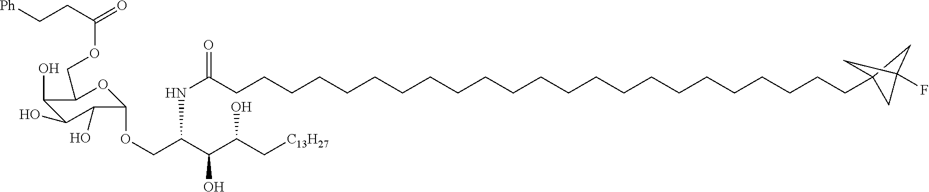

((2R,3R,4S,5R,6S)-6-(((2S,3S,4R)-2-(24-(3-fluorobicyclo[1.1.1]pentan-1-yl)tetracosanamido)-3,4-dihydroxyoctadecyl)oxy)-3,4,5-trihydroxytetrahydro-2H-pyran-2-yl)methyl 3-phenylpropanoate

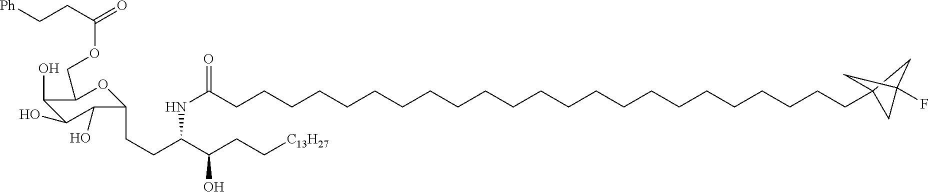

((2R,3R,4R,5R,6R)-6-((3S,4R)-3-(24-(3-fluorobicyclo[1.1.1]pentan-1-yl)tetracosanamido)-4-hydroxynonadecyl)-3,4,5-trihydroxytetrahydro-2H-pyran-2-yl)methyl 3-phenylpropanoate

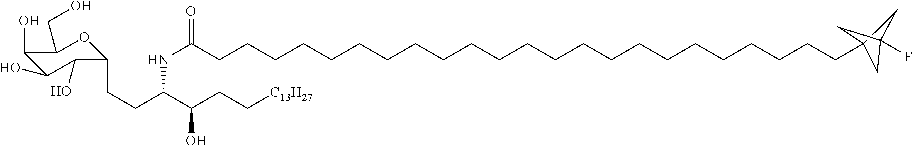

24-(3-fluorobicyclo[1.1.1]pentan-1-yl)-N-((3S,4R)-4-hydroxy-1-((2R,3R,4R,5R,6R)-3,4,5-trihydroxy-6-(hydroxymethyl)tetrahydro-2H-pyran-2-yl) nonadecan-3-yl)tetracosanamide
22. A compound of formula (B):

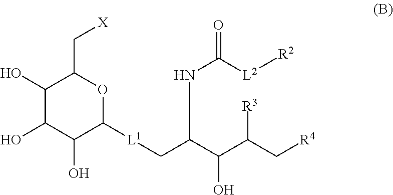

or a pharmaceutically acceptable salt thereof, wherein:
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —O— or —(CH₂)ₘ—;
L² is alkylene;
X is —OH, —OC(O)R⁷, —OC(O)NR⁹R¹⁰, or —N(R^{a})C(O)R¹³, wherein
   when X is OH or —N(R^{a})C(O)R¹³, either
      L¹ is —(CH₂)ₘ—, or
      R³ is hydrogen;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl, wherein the cycloalkyl is optionally substituted with one or more halo;
R¹³ is cycloalkyl optionally substituted with one or more halo; and
each R^{A} is independently aryl.
23. The compound of embodiment 22, or a pharmaceutically acceptable salt thereof, wherein the compound is a compound of formula (B-I):

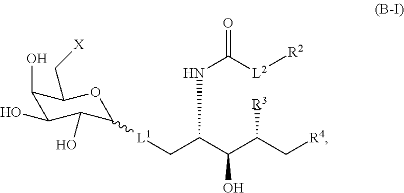

or a pharmaceutically acceptable salt thereof.
24. The compound of embodiment 22 or 23, or a pharmaceutically acceptable salt thereof, wherein the compound is a compound of formula (B-I-i) or (B-I-ii):

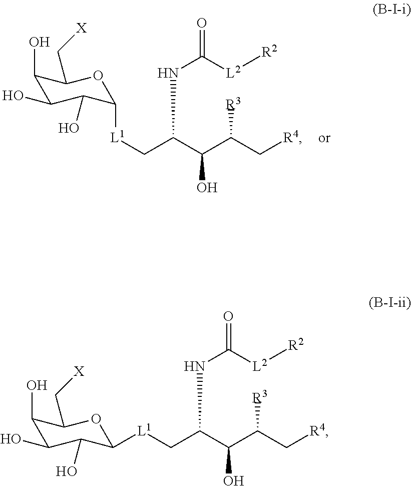

or a pharmaceutically acceptable salt thereof.
25. The compound of any one of embodiments 22 to 24, or a pharmaceutically acceptable salt thereof, wherein the compound is a compound of formula (B-II):

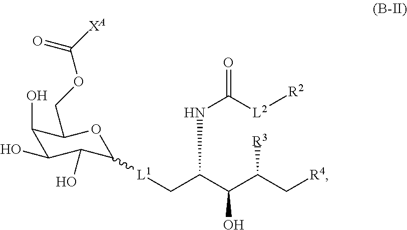

wherein XA is —R⁷ or —NR⁹R¹⁰, or a pharmaceutically acceptable salt thereof.
26. The compound of any one of embodiments 22 to 25, or a pharmaceutically acceptable salt thereof, wherein the compound is a compound of formula (B-III):

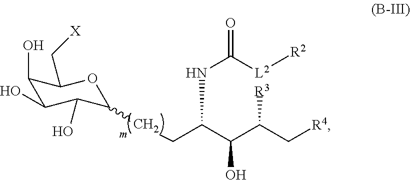

or a pharmaceutically acceptable salt thereof.
27. The compound of any one of embodiments 22 to 26, or a pharmaceutically acceptable salt thereof, wherein R³ is —OH.
28. The compound of any one of embodiments 22 to 25, or a pharmaceutically acceptable salt thereof, wherein the compound is a compound of formula (B-IV):

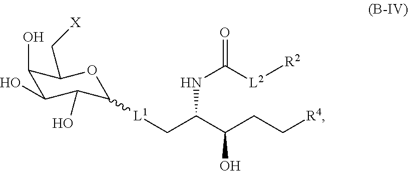

or a pharmaceutically acceptable salt thereof.
29. The compound of any one of embodiments 22 to 28, or a pharmaceutically acceptable salt thereof, wherein R² is

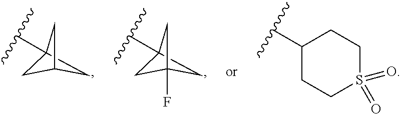 [SMILES: *C12CC(C1)C2 ; *C12CC(F)(C1)C2 ; *C1CCS(=O)(=O)CC1]

30. The compound of any one of embodiments 22 to 29, or a pharmaceutically acceptable salt thereof, wherein R⁴ is C₁₃H₂₇.
31. The compound of any one of embodiments 22 to 25, or 27 to 30, or a pharmaceutically acceptable salt thereof, wherein L¹ is —O—.
32. The compound of any one of embodiments 22 to 31, or a pharmaceutically acceptable salt thereof, wherein L¹ is —(CH₂)ₘ— and m is 1.
33. The compound of any one of embodiments 22 to 32, or a pharmaceutically acceptable salt thereof, wherein L² is —(CH₂) 23.
34. The compound of any one of embodiments 22 to 25, or 27 to 33, or a pharmaceutically acceptable salt thereof, wherein X is —OH.
35. The compound of any one of embodiments 22 to 33, or a pharmaceutically acceptable salt thereof, wherein X is —OC(O)R⁷.
36. The compound of embodiment 35, or a pharmaceutically acceptable salt thereof, wherein R⁷ is

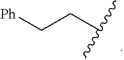 [SMILES: *CCc1ccccc1]

37. The compound of any one of embodiments 22 to 36, or a pharmaceutically acceptable salt thereof, wherein X is —OC(O)NR⁹R¹⁰.

38. The compound of embodiment 37, or a pharmaceutically acceptable salt thereof, wherein one of R⁹ and R¹⁰ is hydrogen and the other of R⁹ and R¹⁰ is

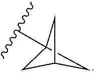 [SMILES: *C12CC(C1)C2]

39. The compound of any one of embodiments 22 to 25, or 27 to 33, or a pharmaceutically acceptable salt thereof, wherein X is —N(R^{a})C(O)R¹³.
40. The compound of embodiment 39, or a pharmaceutically acceptable salt thereof, wherein one of R^{a} is hydrogen and R¹³ is

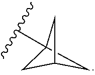 [SMILES: *C12CC(C1)C2]

41. A compound selected from the group consisting of:

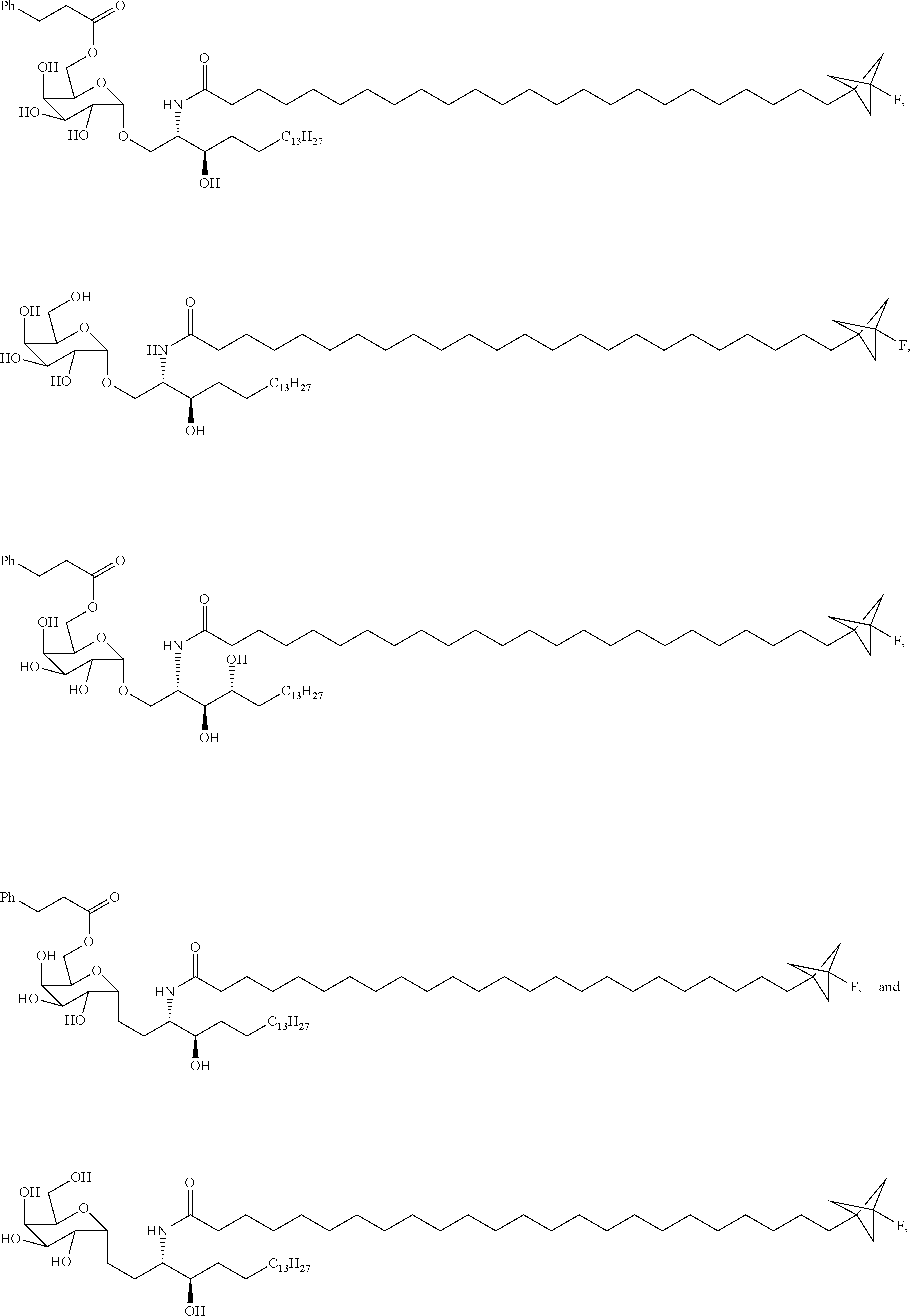

or a pharmaceutically acceptable salt thereof.
42. A compound selected from the group consisting of:

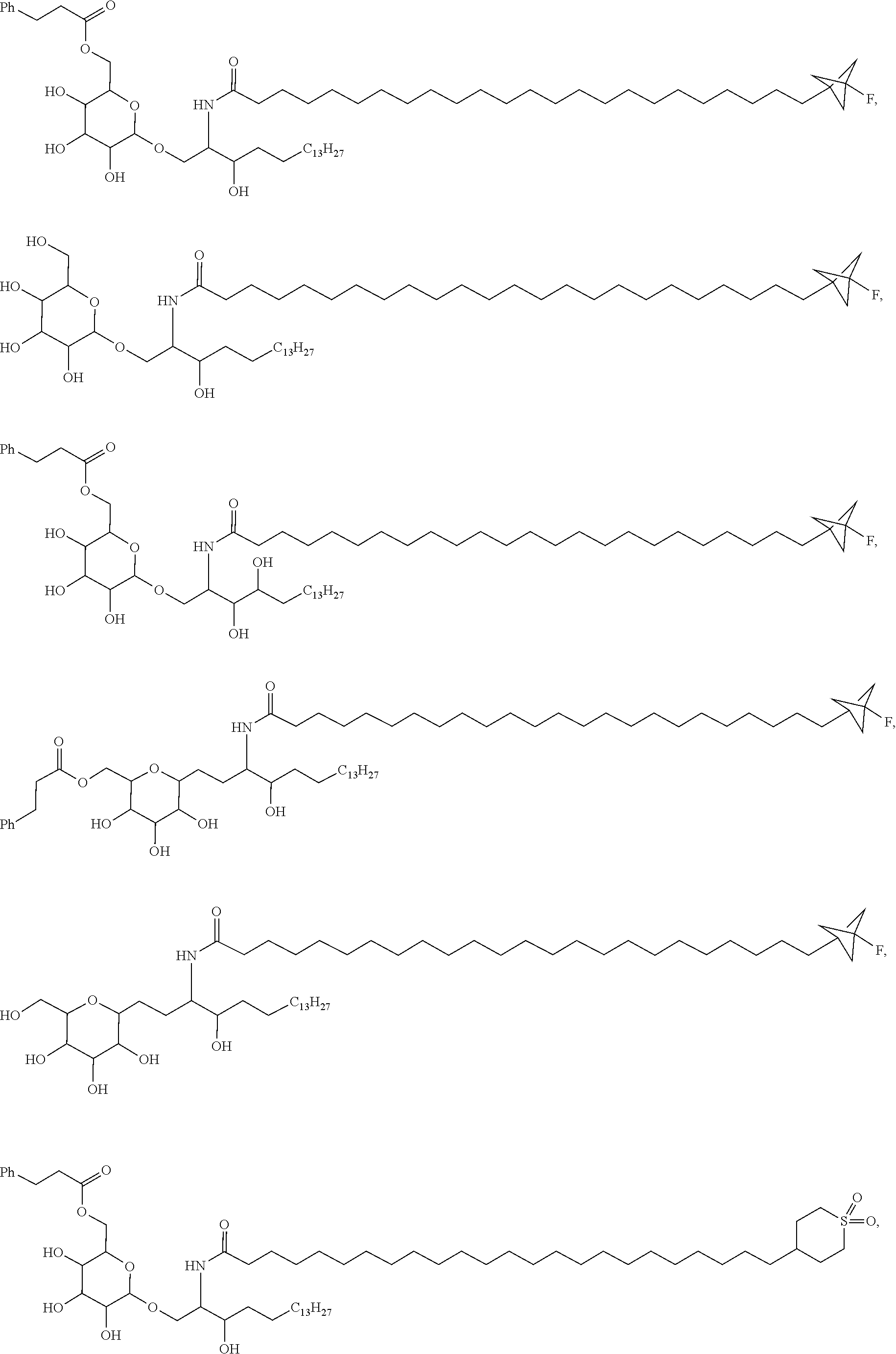

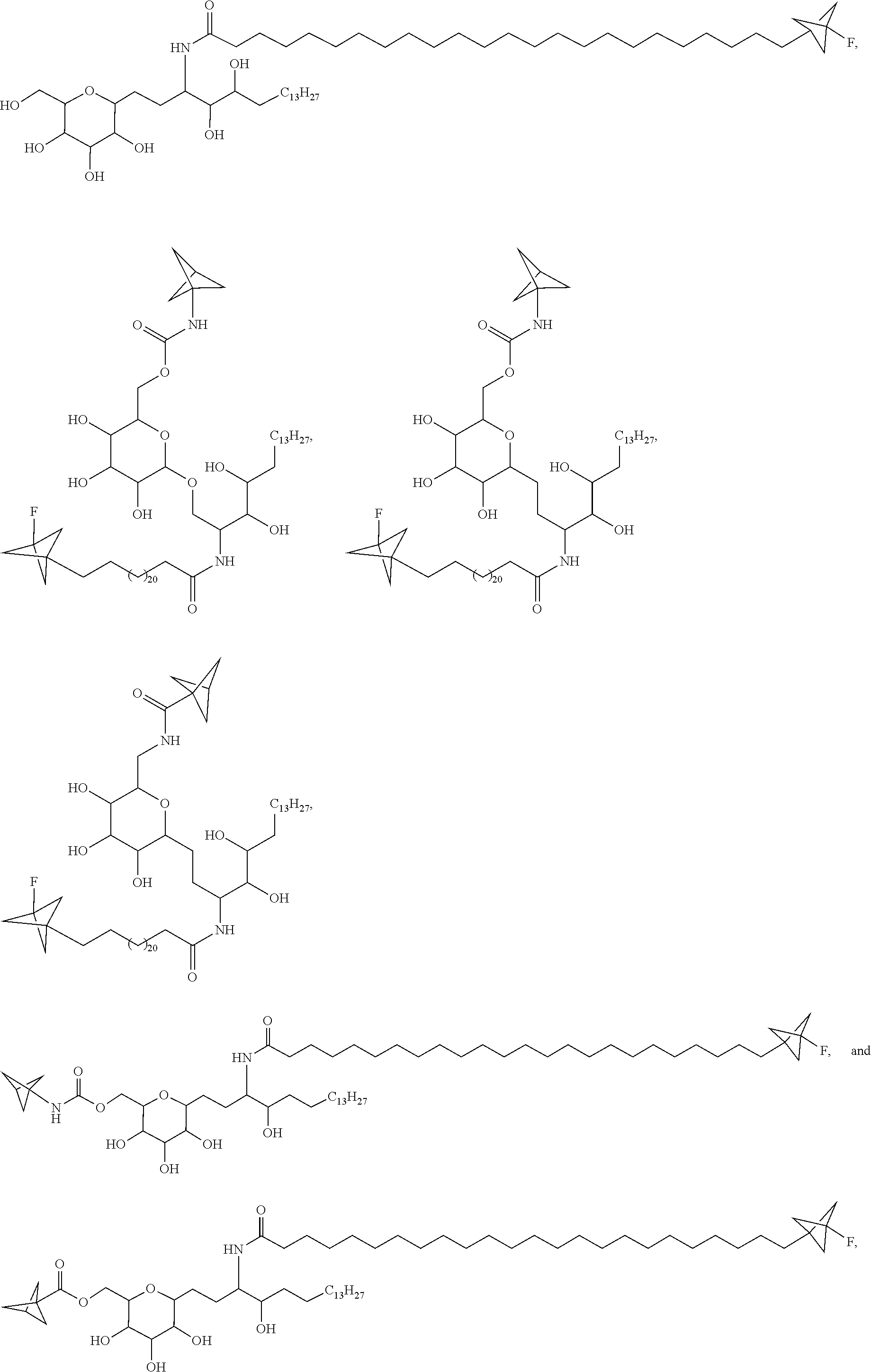

or a pharmaceutically acceptable salt thereof.
43. A compound selected from the group consisting of:

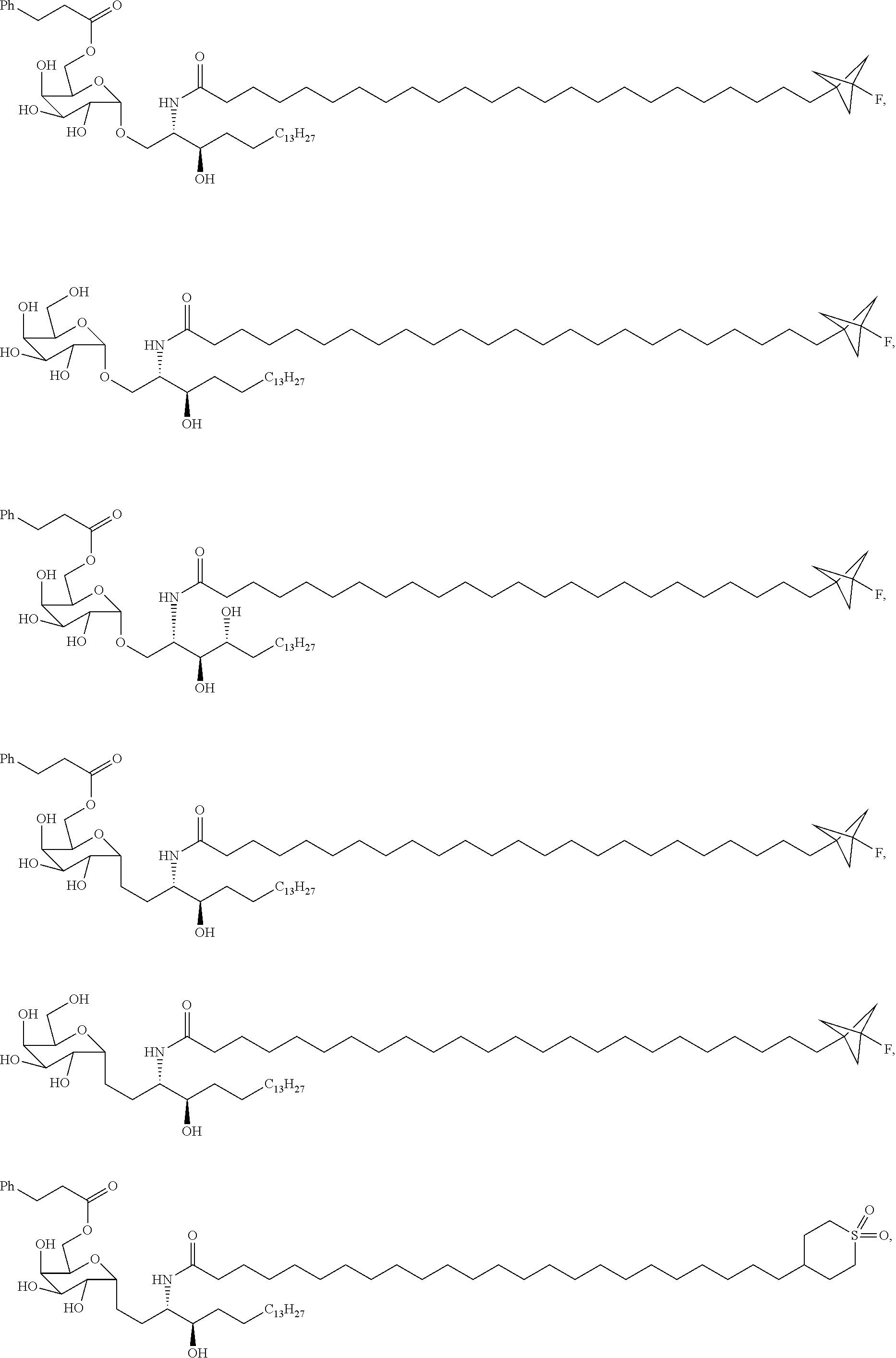

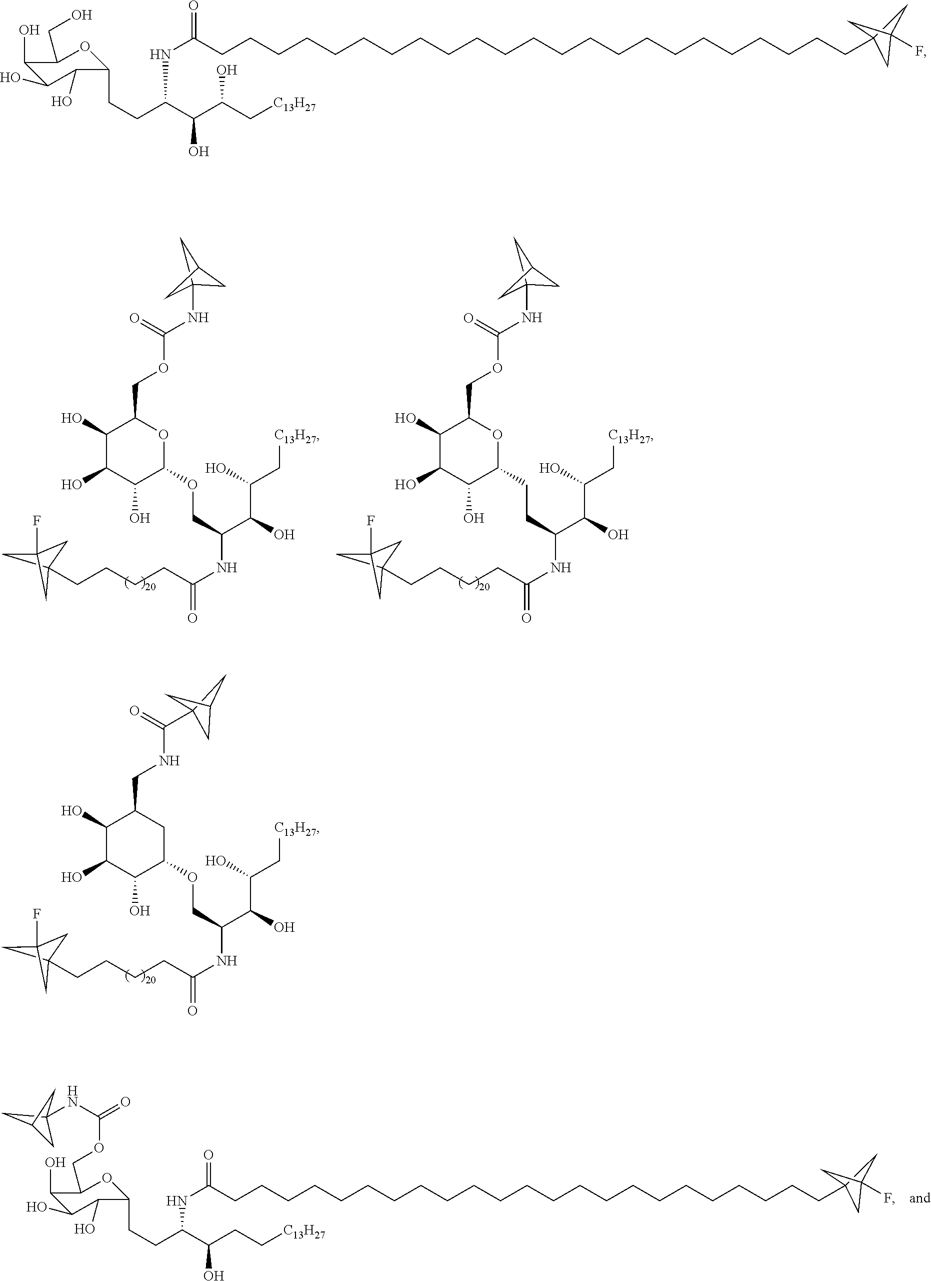

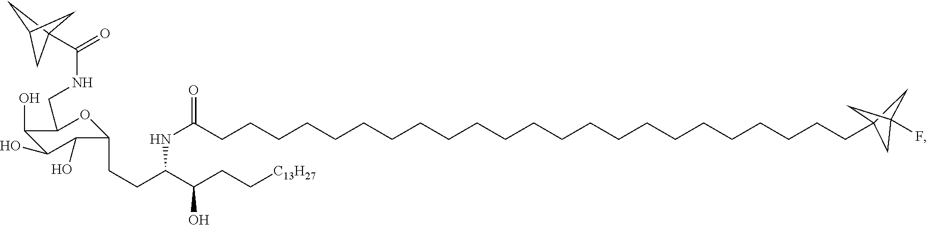

or a pharmaceutically acceptable salt thereof.
44. A pharmaceutical composition, comprising: a compound of any one of embodiments 1 to 43; and at least one pharmaceutically acceptable carrier.
45. A method for activating invariant natural killer T (INKT) cells in a subject in need thereof, comprising administering to the subject a compound of any one of embodiments 1 to 43, or a pharmaceutically composition of embodiment 44.
46. A method, comprising contacting invariant natural killer T (INKT) cells with a compound of any one of embodiments 1 to 43, or a pharmaceutical composition of embodiment 44.
47. The method of embodiment 46, wherein contacting iNKT cells activates the iNKT cells.
48. The method of embodiment 47, wherein the activated iNKT cells reduce the presence of senescent cells.
49. The method of any one of embodiments 46 to 48, wherein contacting the INKT cells occurs in vitro.
50. The method of any one of embodiments 46 to 48, wherein contacting the iNKT cells occurs in vivo.
51. The method according to embodiment 46, wherein contacting comprises administering the compound or composition to a subject.
52. The method according to embodiment 51, wherein the subject has an autoimmune disorder, an allergic disorder, a metabolic disorder, a fibrotic disorder or a combination thereof.
53. The method of embodiment 52, wherein the fibrotic disorder is a disorder of the lung, kidney or liver.
54. The method of embodiment 51, wherein the subject has a disorder selected from type 2 diabetes, diabetic nephropathy, diabetic kidney disease, diabetic bladder dysfunction, diabetic retinopathy, diabetic macular edema, NAFLD, NASH, cancer, an eye disease, age related macular degeneration, heart disease, heart failure, atherosclerosis, hypertension, kidney disease, cardiorenal syndrome, pathogen infection, rheumatoid arthritis, ulcerative colitis, multiple sclerosis, familial hypercholesteremia, giant cell arteritis, idiopathic pulmonary fibrosis, systemic lupus erythematosus, cachexia, Werner syndrome, Fuchs' endothelial dystrophy, glaucoma, cataracts, posterior non-infectious uveitis, chronic obstructive pulmonary disease, systemic sclerosis, pulmonary arterial hypertension, lipodystrophy, sarcopenia, osteoporosis, Duchenne muscular dystrophy, myotonic dystrophy, alopecia, post myocardial infarction, vitiligo, POTS, MCAD, Sjögren's, scleroderma, Hashimoto Disease, ankylosing spondylitis, fibromyalgia, sarcoidosis, hepatitis, Raynaud's Syndrome, mold illness, celiac, Crohn's, inflammatory bowel disease, pemphigus, SPS, PBC/PBC (cholestatic diseases), Psoriatic Arthritis, CIDP, motor neuron disease, GPA, ALS, POAG, myasthenia gravis, presbyopia, or a combination thereof.
55. A method for selectively reducing the presence of or eliminating senescent cells by activating invariant natural killer T (INKT) cells in a subject in need thereof, comprising: administering to the subject a compound of any one of embodiments 1 to 43, or a pharmaceutically composition of embodiment 44.
56. A method of treating a disease, disorder or condition by activating invariant natural killer T (INKT) cells in a subject in need thereof, comprising: administering to the subject a compound of any one of embodiments 1 to 43, or a pharmaceutically composition of embodiment 44.
57. The method of embodiment 56, wherein the disease, disorder or condition is an autoimmune disease, an allergic disease, a metabolic syndrome or disorder, cancer, a pathogen infection, an eye disease, a disease of aging, fibrosis, heart disease, or kidney disease, or any combination thereof.
58. The method of embodiment 57, wherein the fibrosis is lung fibrosis.
59 The method of embodiment 57, wherein the fibrosis is kidney fibrosis.
60. The method of embodiment 57, wherein the fibrosis is liver fibrosis.
61. The method of embodiment 56, wherein the disease, disorder or condition is type 1 diabetes, type 2 diabetes, diabetic nephropathy, diabetic kidney disease, diabetic bladder dysfunction, diabetic retinopathy, diabetic macular edema, fatty liver disease, such as non-alcoholic fatty liver disease/metabolic-associated fatty liver disease (NAFLD/MAFLD), non-alcoholic steatohepatitis/metabolic dysfunction-associated steatohepatitis (NASH/MASH), cancer, an eye disease, age related macular degeneration, heart disease, heart failure, atherosclerosis, hypertension, kidney disease, cardiorenal syndrome, pathogen infection, rheumatoid arthritis, ulcerative colitis, multiple sclerosis, familial hypercholesteremia, giant cell arteritis, idiopathic pulmonary fibrosis (IPF), systemic lupus erythematosus, cachexia, Werner syndrome, Fuchs' endothelial dystrophy, glaucoma, cataracts, posterior non-infectious uveitis, chronic obstructive pulmonary disease, systemic sclerosis, pulmonary arterial hypertension, lipodystrophy, sarcopenia, osteoporosis, Duchenne muscular dystrophy, myotonic dystrophy, alopecia, post myocardial infarction, vitiligo, postural orthostatic tachycardia syndrome (POTS), medium-chain acyl-coenzyme A dehydrogenase deficiency (MCAD), Sjögren's syndrome, scleroderma, Hashimoto Disease, ankylosing spondylitis, fibromyalgia, sarcoidosis, hepatitis, Raynaud's Syndrome, chronic inflammatory response syndrome or mold illness, celiac, Crohn's disease, inflammatory bowel disease, pemphigus, Stiff Person Syndrome (SPS), Primary Biliary Cholangitis (PBC) (cholestatic diseases), psoriatic arthritis, chronic inflammatory demyelinating polyradiculoneuropathy (CIDP), motor neuron disease, granulomatosis with polyangiitis (GPA) (Wegener's disease), amyotrophic lateral sclerosis (ALS), primary open angle glaucoma (POAG), myasthenia gravis, presbyopia, or a combination thereof.
62. The method of embodiment 56, wherein the disease, disorder or condition is Type 1 diabetes.
63. The method of embodiment 56, wherein the disease, disorder or condition is Type 2 diabetes.
64. The method of embodiment 56, wherein the disease, disorder or condition is Idiopathic Pulmonary Fibrosis (IPF).

### EXAMPLES

The presently disclosed subject matter will be better understood by reference to the following Examples, which are provided as exemplary of the invention, and not by way of limitation.

### A. Compound Synthesis and Characterization

### Example A1: Intermediate A1

### Step 1: Synthesis of 8-(1,1-dioxidotetrahydro-4H-thiopyran-4-ylidene) octanoic acid

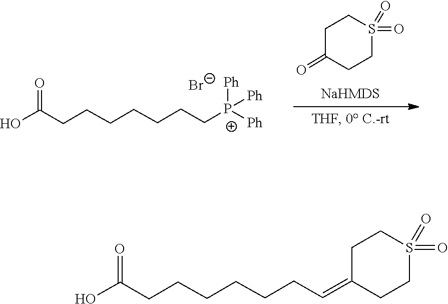 [SMILES: O=C1CCS(=O)(=O)CC1 ; O=C(O)CCCCCCC[P+](c1ccccc1)(c1ccccc1)c1ccccc1 ; [Br-] ; O=C(O)CCCCCCC=C1CCS(=O)(=O)CC1]

To a mixture of (7-carboxyheptyl)triphenylphosphonium bromide (7.86 g, 16.2 mmol, 1.2 equiv) and THF (100 mL) at 0° C. under an atmosphere of N₂ was added NaHMDS, 2M in THF (17 mL, 34 mmol, 2.5 equiv). The mixture was warmed to room temperature (“rt”) and stirred for 1.5 h, then tetrahydro-4H-thiopyran-4-one 1,1-dioxide (2 g, 13.5 mmol, 1.0 equiv) was added to the mixture and the reaction was stirred at rt overnight. H₂O (50 mL) was added, and the mixture was acidified to pH ˜5 with 1N HCl, then extracted with EtOAc (3×100 mL). The combined organic layers were washed with brine (50 mL), dried over anhydrous Na₂SO₄ and concentrated under vacuum. The residue was purified by column chromatography on silica gel eluting with EtOAc/petroleum ether (1:1) to give the product (800 mg, 21%) as a solid. m/z=mass calcd. for C₁₃H₂₂O₄S: 274, found: 273 [M−H]⁻.

### Step 2: Synthesis of 8-(1,1-dioxidotetrahydro-2H-thiopyran-4-yl) octanoic acid

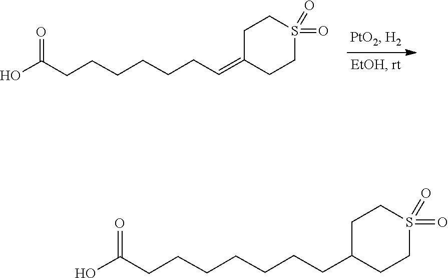 [SMILES: O=C(O)CCCCCCC=C1CCS(=O)(=O)CC1 ; O=C(O)CCCCCCCC1CCS(=O)(=O)CC1]

A mixture of 8-(1,1-dioxidotetrahydro-4H-thiopyran-4-ylidene) octanoic acid (600 mg, 2.2 mmol, 1.0 equiv), EtOH (50 mL) and PtO₂ (60 mg) was stirred under an atmosphere of H₂ for 1.5 h (balloon). The mixture was filtered through a pad of celite and the filtrate was concentrated under vacuum to give the product (510 mg, 84%) as a solid. m/z=mass calcd. for C₁₃H₂₄O₄S: 276, found: 275 [M−H]⁻.

### Step 3: Synthesis of 4-(8-hydroxyoctyl)tetrahydro-2H-thiopyran 1,1-dioxide

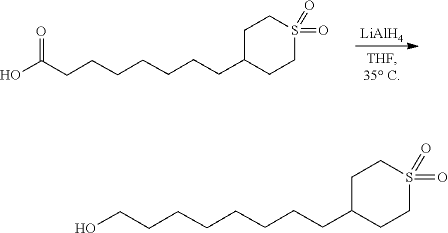 [SMILES: O=C(O)CCCCCCCC1CCS(=O)(=O)CC1 ; "LiAlH4" ; O=S1(=O)CCC(CCCCCCCCO)CC1]

To a mixture of 8-(1,1-dioxidotetrahydro-2H-thiopyran-4-yl) octanoic acid (510 mg, 1.85 mmol, 1.0 equiv) and THF (20 mL) at rt under an atmosphere of N₂ was added LiAlH₄ (140 mg, 3.7 mmol, 2.0 equiv). The mixture was heated to 35° C. and stirred for 1 h, then cooled to 0° C. and quenched with aqueous Na₂SO₄. The mixture was dried over Na₂SO₄, and concentrated under vacuum. The residue was purified by column chromatography on silica gel eluting with EtOAc/petroleum ether (1:2) to give the product (400 mg, 82%) as a solid.

### Step 4: Synthesis of 8-(1,1-dioxidotetrahydro-2H-thiopyran-4-yl) octanal

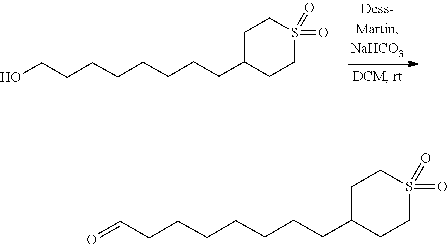 [SMILES: O=S1(=O)CCC(CCCCCCCCO)CC1 ; O=CCCCCCCCC1CCS(=O)(=O)CC1]

To a mixture of 4-(8-hydroxyoctyl)tetrahydro-2H-thiopyran 1,1-dioxide (300 mg, 1.14 mmol, 1.00 equiv), NaHCO₃ (384 mg, 4.57 mmol, 4.00 equiv) and DCM (10 mL) at rt was added Dess-Martin periodinane (969 mg, 2.3 mmol, 2.0 equiv). The mixture was stirred at rt for 2 h, then diluted with H₂O (15 mL) and extracted with DCM (3×20 mL). The combined organic layers were washed with brine (20 mL), dried over anhydrous Na₂SO₄ and concentrated under vacuum. The residue was purified by column chromatography on silica gel eluting with EtOAc/petroleum ether (1:3) to give the product (200 mg, 67%) as a solid.

### Step 5: Synthesis of (E)-24-(1,1-dioxidotetrahydro-2H-thiopyran-4-yl)tetracos-16-enoic acid

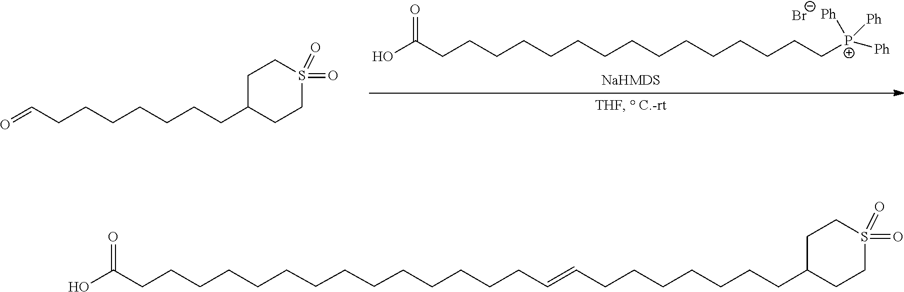 [SMILES: O=CCCCCCCCC1CCS(=O)(=O)CC1 ; O=C(O)CCCCCCCCCCCCCCC[P+](c1ccccc1)(c1ccccc1)c1ccccc1 ; [Br-] ; O=C(O)CCCCCCCCCCCCCC/C=C/CCCCCCCC1CCS(=O)(=O)CC1]

To a mixture of (15-carboxypentadecyl)triphenylphosphonium bromide (551 mg, 0.99 mmol, 1.2 equiv) and THF (10 mL) at 0° C. under an atmosphere of N₂ was added NaHMDS, 2M in THF (1.0 mL, 2.0 mmol, 2.5 equiv). The mixture was stirred at 0° C. for 1 h, then 8-(1,1-dioxidotetrahydro-2H-thiopyran-4-yl) octanal (200 mg, 0.77 mmol, 1.0 equiv) was added at 0° C. The mixture was warmed to rt and stirred overnight, then diluted with H₂O (10 mL), acidified to pH ˜5 with 1N HCl and extracted with EtOAc (3×20 mL). The combined organic layers were washed with brine (20 mL), dried over anhydrous Na₂SO₄ and concentrated under vacuum. The residue was purified by column chromatography on silica gel eluting with EtOAc/petroleum ether (1:2) to give the product (150 mg, 39%), as a solid. m/z=mass calcd. for C₂₉H₅₄O₄S: 498, found: 497 [M−H]⁻.

### Step 6: Synthesis of 24-(1,1-dioxidotetrahydro-2H-thiopyran-4-yl)tetracosanoic acid (Intermediate A-1)

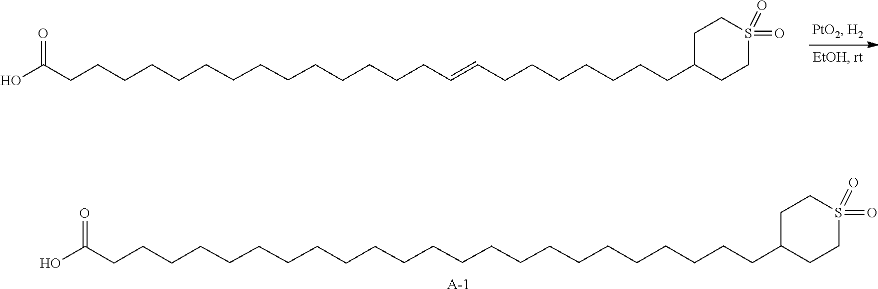

A mixture of (E)-24-(1,1-dioxidotetrahydro-2H-thiopyran-4-yl)tetracos-16-enoic acid (150 mg, 0.3 mmol, 1.0 equiv), EtOH (10 mL) and PtO₂ (20 mg) was stirred under an atmosphere of H₂ (balloon) for 1.5 h. The mixture was filtered through a pad of celite and the filtrate was concentrated under vacuum to give the product (120 mg, 79%) as a solid. m/z=mass calcd. for C₂₉H₅₆O₄S: 500, found: 499 [M−H]⁻.

### Example A2: Compound I-1

### Synthesis of ((2R,3R,4S,5R,6S)-6-(((2S,3R)-2-(24-(3-fluorobicyclo[1.1.1]pentan-1-yl)tetracosanamido)-3-hydroxyoctadecyl)oxy)-3,4,5-trihydroxytetrahydro-2H-pyran-2-yl)methyl 3-phenylpropanoate (Compound I-1)

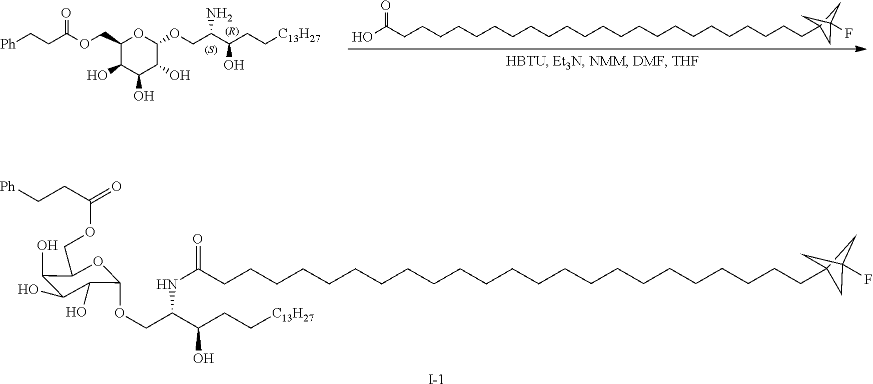

The compound 24-(3-fluorobicyclo[1.1.1]pentan-1-yl)tetracosanoic acid may be obtained from any commercially available sources or prepared according to any methods known in the art. For example, the synthesis of this compound is described in WO2022/187141, which is hereby incorporated by reference, including specifically with respect to page 234 line 14-page 236, line 15 and page 230 line-page 231, line 7, in which the synthesis and characterization of the compound is provided.

To a mixture of 24-(3-fluorobicyclo[1.1.1]pentan-1-yl)tetracosanoic acid (76 mg, 0.168 mmol, 1.0 equiv) and ((2R,3R,4S,5R,6S)-6-(((2S,3R)-2-amino-3-hydroxyoctadecyl)oxy)-3,4,5-trihydroxytetrahydro-2H-pyran-2-yl)methyl 3-phenylpropanoate (100 mg, 0.17 mmol, 1.0 equiv) in DMF (3 mL), THF (3 mL) at rt under an atmosphere of N₂, were added HBTU (95 mg, 0.252 mmol, 1.5 equiv), Et₃N(0.1 mL), and NMM (0.1 mL). The mixture was stirred at rt for 16 h, then diluted with H₂O and extracted with EtOAc. The organic layer was concentrated under vacuum and the residue was purified by column chromatography on silica gel using DCM/MeOH (9:1). The resulting solid was washed with CH₃CN and dried to give the product (9 mg, 4%) as a solid. m/z=mass calcd. for C₆₂H₁₀₈FNO₉:1029.80, found: 1030.75 [M+H]⁺; 1H NMR (400 MHZ, DMSO-d₆) δ 7.56 (d, J=8.9 Hz, 1H), 7.22-7.26 (m, 5H), 4.50-4.73 (m, 4H), 4.24 (dd, J=8.0, 5.3 Hz, 1H), 4.08 (d, J=7.5 Hz, 2H), 3.83 (d, J=6.2 Hz, 1H), 3.73 (s, 1H), 3.40-3.68 (m, 5H), 3.31 (s, 1H), 2.84 (t, J=7.8 Hz, 2H), 2.60-2.72 (m, 2H), 2.23-2.35 (m, 1H), 2.04 (dd, J=14.9, 7.7 Hz, 2H), 1.87 (d, J=2.8 Hz, 6H), 1.54-1.68 (m, 2H), 1.38 (d, J=16.9 Hz, 6H), 1.23 (t, J=3.8 Hz, 63H), 0.89-0.83 (m, 3H).

### Example A3: Compound I-2

### Synthesis of 24-(3-fluorobicyclo[1.1.1]pentan-1-yl)-N-((2S,3R)-3-hydroxy-1-(((2S,3R,4S,5R,6R)-3,4,5-trihydroxy-6-(hydroxymethyl)tetrahydro-2H-pyran-2-yl)oxy) octadecan-2-yl)tetracosanamide (Compound I-2)

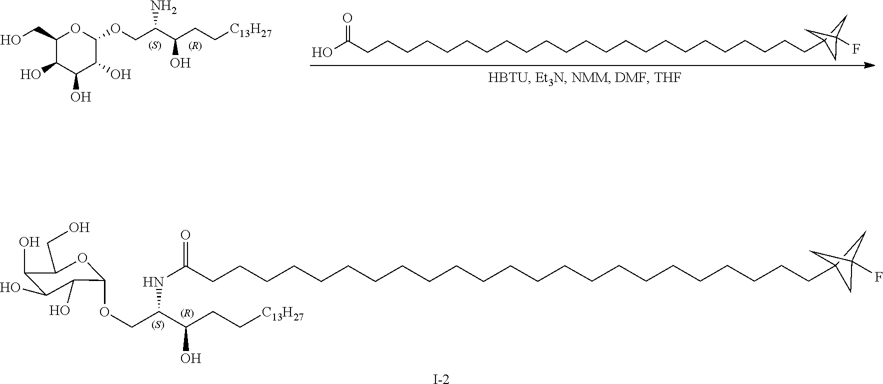

To a mixture of 24-(3-fluorobicyclo[1.1.1]pentan-1-yl)tetracosanoic acid (100 mg, 0.22 mmol, 1.0 equiv) and (2S,3R,4S,5R,6R)-2-(((2S,3R)-2-amino-3-hydroxyoctadecyl)oxy)-6-(hydroxymethyl)tetrahydro-2H-pyran-3,4,5-triol (102 mg, 0.22 mmol, 1.0 equiv) in DMF (3 mL), THF (3 mL) at rt under an atmosphere of N₂ were added HBTU (125 mg, 0.332 mmol, 1.5 equiv), Et₃N(0.1 mL), and NMM (0.1 mL). The mixture was stirred at rt for 16 h, then diluted with H₂O (10 mL) and extracted EtOAc (30 mL×3). The combined organic layers were concentrated under vacuum and the residue was purified by column chromatography on silica gel using DCM/MeOH (9:1). The resulting solid was washed with CH₃CN and dried to give the product (14.6 mg, 6%) as a solid. m/z=mass calcd. for C₅₃H₁₀₀FNO₈: 897.74, found: 898.65 [M+H]⁺; 1H NMR (300 MHz, CD₃OD) δ 3.58-3.97 (m, 10H), 2.22 (t, J=7.5 Hz, 2H), 1.85 (d, J=2.7 Hz, 3H), 1.59-1.61 (m, 6H), 1.27-1.30 (s, 67H), 0.90 (t, J=6.5 Hz, 6H).

### Example A4: Compound I-3

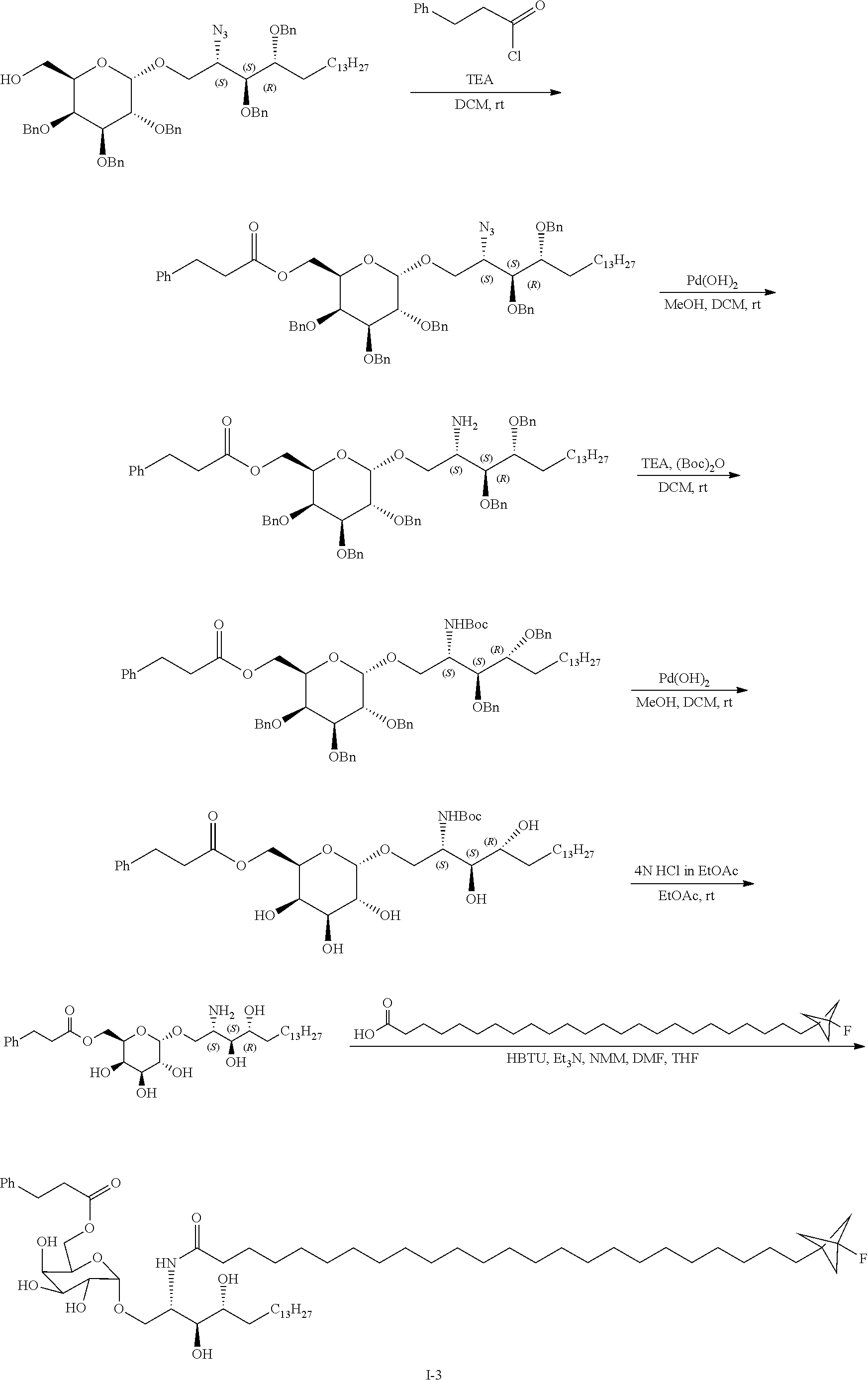

### Step 1: Synthesis of ((2R,3S,4S,5R,6S)-6-(((2S,3S,4R)-2-azido-3,4-bis(benzyloxy) octadecyl)oxy)-3,4,5-tris(benzyloxy) tetrahydro-2H-pyran-2-yl)methyl 3-phenylpropanoate

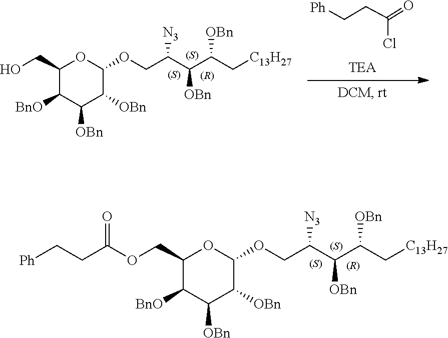

To a mixture of ((2R,3S,4S,5R,6S)-6-(((2S,3S,4R)-2-azido-3,4-bis(benzyloxy) octadecyl)oxy)-3,4,5-tris(benzyloxy) tetrahydro-2H-pyran-2-yl) methanol (1 g, 1.05 mmol, 1 equiv) in DCM (30 mL) at 0° C. was added Et₃N(0.32 g, 3.1 mmol, 3 equiv) and benzenepropanoyl chloride (0.26 g, 1.54 mmol, 1.5 equiv). The mixture was warmed to rt and stirred for 3 h, then diluted with NaHCO₃(aq., 20 mL) and extracted with DCM (3×30 mL). The combined organic layers were dried over Na₂SO₄ and concentrated under vacuum. The residue was purified by column chromatography on silica gel eluting with EtOAc/petroleum ether (1:3) to give the product (800 mg, 70%) as an oil. m/z=mass calcd. for C₆₈H₈₅N₃O₉:1088, found: 1111 [M+Na]⁺.

### Step 2: Synthesis of ((2R,3S,4S,5R,6S)-6-(((2S,3S,4R)-2-amino-3,4-bis(benzyloxy) octadecyl)oxy)-3,4,5-tris(benzyloxy) tetrahydro-2H-pyran-2-yl)methyl 3-phenylpropanoate

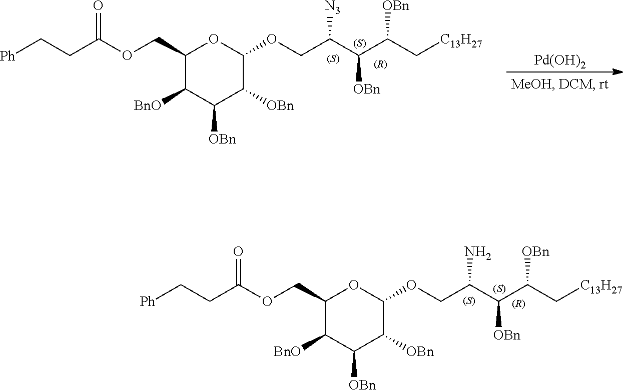

A mixture of ((2R,3S,4S,5R,6S)-6-(((2S,3S,4R)-2-azido-3,4-bis(benzyloxy) octadecyl)oxy)-3,4,5-tris(benzyloxy) tetrahydro-2H-pyran-2-yl)methyl 3-phenylpropanoate (800 mg, 0.74 mmol, 1 equiv), MeOH (20 mL), DCM (4 mL) and Pd(OH)₂ (0.1 g) was stirred at rt under an atmosphere of H₂ for 1 h. The mixture was filtered and the filtrate was concentrated under vacuum to give the product (700 mg, 89%) as an oil. m/z=mass calcd. for C₆₈H₈₇NO₉:1062, found: 1085 [M+Na]⁺.

### Step 3: Synthesis of ((2R,3S,4S,5R,6S)-3,4,5-tris(benzyloxy)-6-(((2S,3S,4R)-3,4-bis(benzyloxy)-2-((tert-butoxycarbonyl)amino) octadecyl)oxy)tetrahydro-2H-pyran-2-yl)methyl 3-phenylpropanoate

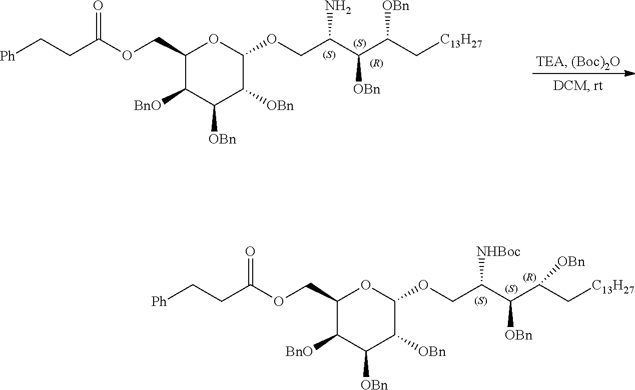

To a mixture of ((2R,3S,4S,5R,6S)-6-(((2S,3S,4R)-2-amino-3,4-bis(benzyloxy) octadecyl)oxy)-3,4,5-tris(benzyloxy) tetrahydro-2H-pyran-2-yl)methyl 3-phenylpropanoate (700 mg, 0.66 mmol, 1 equiv) in DCM (20 mL) was added Et₃N(200 mg, 1.98 mmol, 3 equiv) and (Boc) 20 (216 mg, 0.99 mmol, 1.5 equiv). The mixture was stirred at rt for 4 h, then diluted with NaHCO₃(aq., 20 mL) and extracted with DCM (3×30 mL). The combined organic layers were dried over Na₂SO₄ and concentrated under vacuum. The residue was purified by column chromatography on silica gel eluting with EtOAc/petroleum ether (1:3) to give the product (650 mg, 85%) as an oil. m/z=mass calcd. for C₇₃H₉₅NO₁₁:1162, found: 1185 [M+Na]⁺.

### Step 4: Synthesis of ((2R,3R,4S,5R,6S)-6-(((2S,3S,4R)-2-((tert-butoxycarbonyl)amino)-3,4-dihydroxyoctadecyl)oxy)-3,4,5-trihydroxytetrahydro-2H-pyran-2-yl)methyl 3-phenylpropanoate

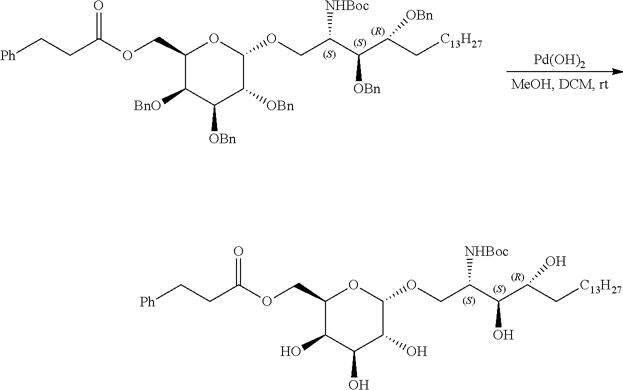

A mixture of ((2R,3S,4S,5R,6S)-3,4,5-tris(benzyloxy)-6-(((2S,3S,4R)-3,4-bis(benzyloxy)-2-((tert-butoxycarbonyl)amino) octadecyl)oxy)tetrahydro-2H-pyran-2-yl)methyl 3-phenylpropanoate (650 mg, 0.56 mmol, 1 equiv) and Pd(OH)₂ (80 mg) in MeOH (20 mL) and DCM (4 mL) was stirred at rt overnight under an atmosphere of H₂. The mixture was filtered and concentrated under vacuum to give the product (300 mg, 75%) as a solid. m/z=mass calcd. for C₃₈H₆₅NO₁₁:711, found: 712 [M+H]⁺.

### Step 5: Synthesis of ((2R,3R,4S,5R,6S)-6-(((2S,3S,4R)-2-amino-3,4-dihydroxyoctadecyl)oxy)-3,4,5-trihydroxytetrahydro-2H-pyran-2-yl)methyl 3-phenylpropanoate

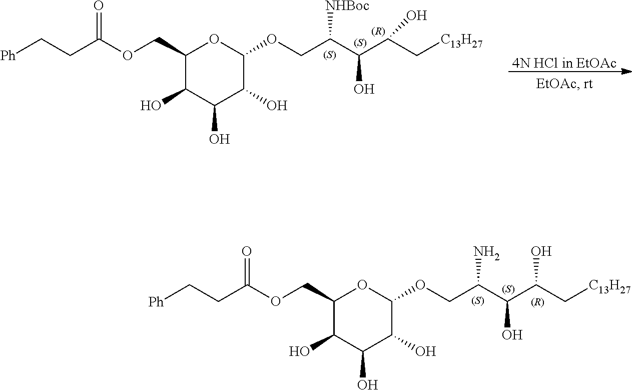

To a mixture of ((2R,3R,4S,5R,6S)-6-(((2S,3S,4R)-2-((tert-butoxycarbonyl)amino)-3,4-dihydroxyoctadecyl)oxy)-3,4,5-trihydroxytetrahydro-2H-pyran-2-yl)methyl 3-phenylpropanoate (300 mg, 0.42 mmol, 1 equiv) in EtOAc (5 mL) at 0° C. was added HCl, 4N in EtOAc (5 mL). The reaction was warmed to rt for 4 h, then concentrated under vacuum to give the product (230 mg, 89%) as a solid. m/z=mass calcd. for C₃₃H₅₇NO₉:611, found: 612 [M+H]⁺.

### Step 6: Synthesis of ((2R,3R,4S,5R,6S)-6-(((2S,3S,4R)-2-(24-(3-fluorobicyclo[1.1.1]pentan-1-yl)tetracosanamido)-3,4-dihydroxyoctadecyl)oxy)-3,4,5-trihydroxytetrahydro-2H-pyran-2-yl)methyl 3-phenylpropanoate (Compound I-3)

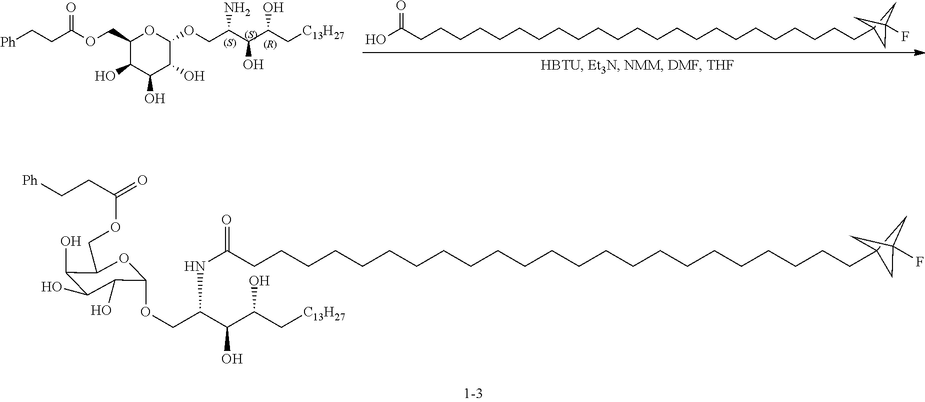

A mixture of 24-(3-fluorobicyclo[1.1.1]pentan-1-yl)tetracosanoic acid (60 mg, 0.13 mmol, 1 equiv), ((2R,3R,4S,5R,6S)-6-(((2S,3S,4R)-2-amino-3,4-dihydroxyoctadecyl)oxy)-3,4,5-trihydroxytetrahydro-2H-pyran-2-yl)methyl 3-phenylpropanoate (81 mg, 0.13 mmol, 1.0 equiv), Et₃N(0.1 mL) and NMM (0.1 mL) in DMF (4 mL) and THF (4 mL) was stirred at rt overnight. The mixture was diluted with brine (30 mL) and extracted with THF (3×30 mL). The combined organic layers were concentrated under vacuum and the residue was purified by column chromatography on silica gel eluting with MeOH/DCM (1:9), and then purified further by preparative HPLC (Agela C₁₈ reverse column; eluent: 5% to 80% (v/v) ACN and H₂O, then THF) to give the product (22.9 mg, 16%) as a solid. m/z=mass calcd. for C₆₂H₁₀₈FNO₁₀:1045. 80, found: 1046.70 [M+H]⁺; 1H NMR (400 MHZ, DMSO-d₆) δ 7.60 (d, J=8.6 Hz, 1H), 7.11-7.31 (m, 5H), 4.51-4.72 (m, 4H), 4.39 (d, J=7.3 Hz, 1H), 4.23 (d, J=6.8 Hz, 1H), 4.01-4.10 (m, 2H), 3.91-4.01 (m, 1H), 3.77-3.85 (m, 1H), 3.41-3.70 (m, 6H), 3.21-3.28 (m, 1H), 2.83 (t, J=7.7 Hz, 2H), 2.59 (t, J=7.7 Hz, 2H), 1.98-2.10 (m, 2H), 1.86 (d, J=2.7 Hz, 6H), 1.52-1.61 (m, 2H), 1.37-1.49 (m, 5H), 1.20-1.25 (m, 63H), 0.85 (t, J=6.4 Hz, 3H).

### Example A5: Compound I-4

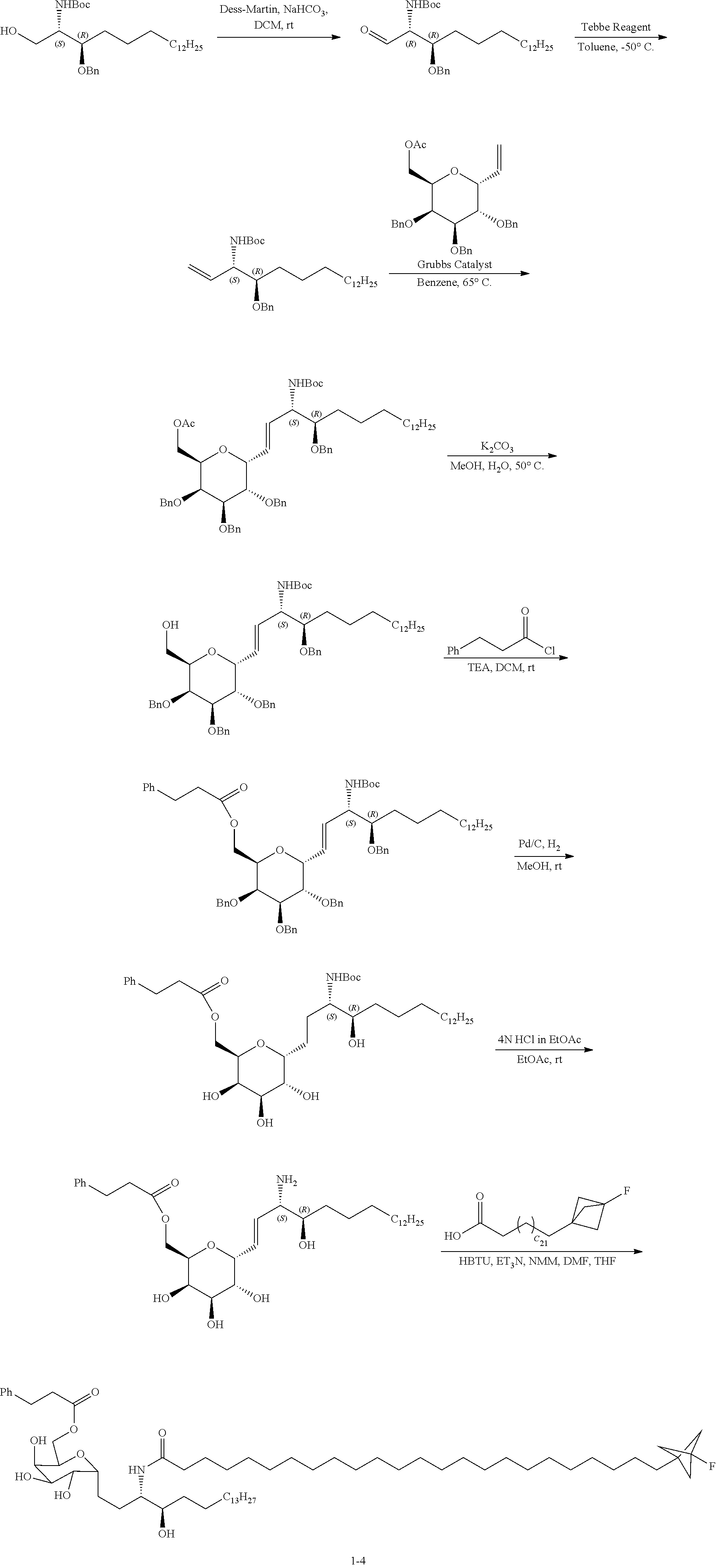

### Step 1: Synthesis of tert-butyl ((2R,3R)-3-(benzyloxy)-1-oxooctadecan-2-yl) carbamate

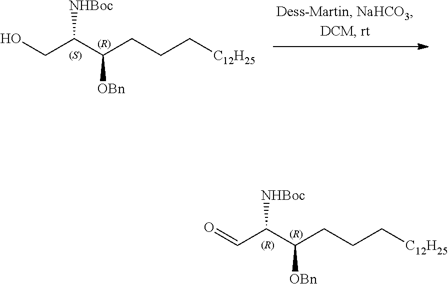

To a mixture of tert-butyl ((2S,3R)-3-(benzyloxy)-1-hydroxyoctadecan-2-yl) carbamate (1 g, 2.0 mmol, 1.0 equiv) in DCM (10 mL) at rt was added NaHCO₃ (683 mg, 8.14 mmol, 4.0 equiv) and Dess-Martin periodinane (1.7 g, 4.1 mmol, 2.0 equiv). The mixture was stirred at rt for 2 h, then diluted with H₂O (50 mL) and extracted with DCM (3×50 mL). The combined organic layers were washed with brine (50 mL), dried over anhydrous Na₂SO₄ and concentrated under vacuum. The residue was purified by column chromatography on silica gel eluting with EtOAc/petroleum ether (1:3) to give the product (600 mg, 60%) as an oil. m/z=mass calcd. for C₃₀H₅₁NO₄:489, found: 490 [M+H]⁺.

### Step 2: Synthesis of tert-butyl ((3S,4R)-4-(benzyloxy) nonadec-1-en-3-yl) carbamate

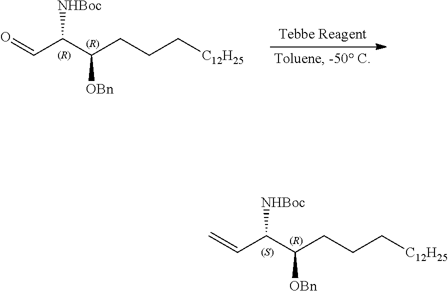

To a mixture of tert-butyl ((2R,3R)-3-(benzyloxy)-1-oxooctadecan-2-yl) carbamate (600 mg, 1.23 mmol, 1.0 equiv) and toluene (10 mL) at −50° C. under an atmosphere of N₂ was added Tebbe reagent, 0.5 M in toluene (3.7 mL, 1.84 mmol, 1.5 equiv). The mixture was stirred at −50° C. for 2 h, then another aliquot of Tebbe reagent, 0.5 M in toluene (3.7 mL, 1.84 mmol, 1.5 equiv) was added. The mixture was warmed to rt and stirred for 16 h, then diluted with H₂O (30 mL) and extracted with EtOAc (3×30 mL). The combined organic layers were washed with brine (30 mL), dried over anhydrous Na₂SO₄ and concentrated under vacuum. The residue was purified by column chromatography on silica gel eluting with EtOAc/petroleum ether (1:3) to give the product (360 mg, 60%) as an oil. m/z=mass calcd. for C₃₁H₅₃NO₃:487, found: 488 [M+H]⁺.

### Step 3: Synthesis of ((2R,3S,4R,5S,6R)-3,4,5-tris(benzyloxy)-6-((3S,4R,E)-4-(benzyloxy)-3-((tert-butoxycarbonyl)amino) nonadec-1-en-1-yl)tetrahydro-2H-pyran-2-yl)methyl acetate

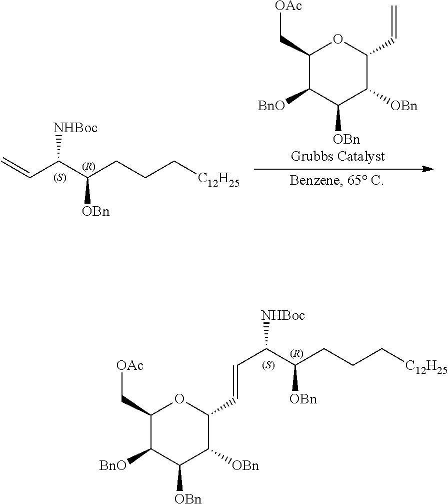

To a mixture of tert-butyl ((3S,4R)-4-(benzyloxy) nonadec-1-en-3-yl) carbamate (360 mg, 0.74 mmol, 1.0 equiv) and ((2R,3S,4R,5S,6R)-3,4,5-tris(benzyloxy)-6-vinyltetrahydro-2H-pyran-2-yl)methyl acetate (11.1 g, 2.21 mmol, 3.0 equiv) in benzene (20 mL) under an atmosphere of N₂ was added Grubbs Catalyst (1.25 g, 1.48 mmol, 2.0 equiv). The mixture was heated to 65° C. and stirred for 48 h, then concentrated under vacuum. The residue was purified by column chromatography on silica gel eluting with EtOAc/petroleum ether (1:4) to give the product (510 mg, crude) as an oil. LC/MS: mass calcd. for C₆₀H₈₃NO₉:962, found: 985 [M+Na]⁺.

### Step 4: Synthesis of tert-butyl ((3S,4R,E)-4-(benzyloxy)-1-((2R,3S,4R,5S,6R)-3,4,5-tris(benzyloxy)-6-(hydroxymethyl)tetrahydro-2H-pyran-2-yl) nonadec-1-en-3-yl) carbamate

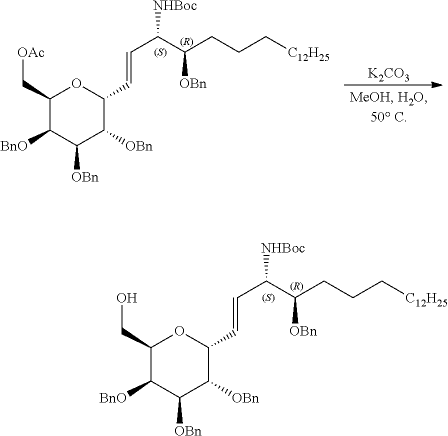

A mixture of ((2R,3S,4R,5S,6R)-3,4,5-tris(benzyloxy)-6-((3S,4R,E)-4-(benzyloxy)-3-((tert-butoxycarbonyl)amino) nonadec-1-en-1-yl)tetrahydro-2H-pyran-2-yl)methyl acetate (510 mg, crude), K₂CO₃ (738 mg), MeOH (15 mL) and H₂O (5 mL) was stirred at 50° C. overnight. After cooling, the mixture was diluted with H₂O (30 mL) and extracted with EtOAc (3×30 mL). The combined organic layers were washed with brine (50 mL), dried over anhydrous Na₂SO₄ and concentrated under vacuum. The residue was purified by column chromatography on silica gel eluting with EtOAc/petroleum ether (1:3) to give the product (445 mg, crude) as an oil. m/z=mass calcd. for C₅₈H₈₁NO₈:920, found: 943 [M+Na]⁺.

### Step 5: Synthesis of ((2R,3S,4R,5S,6R)-3,4,5-tris(benzyloxy)-6-((3S,4R,E)-4-(benzyloxy)-3-((tert-butoxycarbonyl)amino) nonadec-1-en-1-yl)tetrahydro-2H-pyran-2-yl)methyl 3-phenylpropanoate

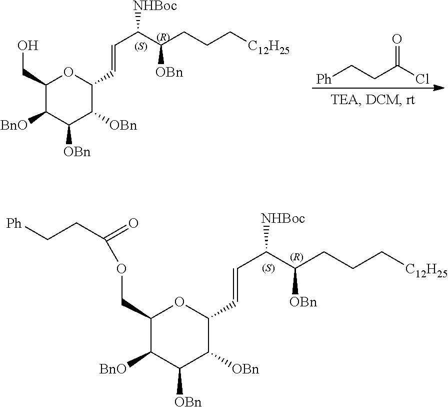

To a mixture of tert-butyl ((3S,4R,E)-4-(benzyloxy)-1-((2R,3S,4R,5S,6R)-3,4,5-tris(benzyloxy)-6-(hydroxymethyl)tetrahydro-2H-pyran-2-yl) nonadec-1-en-3-yl) carbamate (300 mg, crude), Et₃N and DCM (5 mL) at rt was added benzenepropanoyl chloride (165 mg). The mixture was stirred at rt overnight, then diluted with H₂O (20 mL) and extracted with EtOAc (3×20 mL). The combined organic layers were washed with brine (20 mL), dried over anhydrous Na₂SO₄ and concentrated under vacuum. The residue was purified by column chromatography on silica gel eluted with EtOAc/petroleum ether (1:1) to give the product (300 mg) as an oil. m/z=mass calcd. for C₆₇H₈₉NO₉:1052, found: 1075 [M+Na]⁺.

### Step 6: Synthesis of ((2R,3R,4R,5R,6R)-6-((3S,4R)-3-((tert-butoxycarbonyl)amino)-4-hydroxynonadecyl)-3,4,5-trihydroxytetrahydro-2H-pyran-2-yl)methyl 3-phenylpropanoate

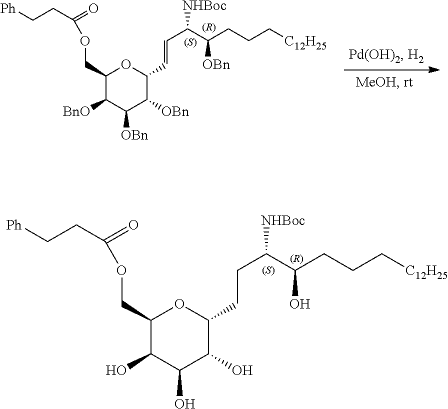

A mixture of ((2R,3S,4R,5S,6R)-3,4,5-tris(benzyloxy)-6-((3S,4R,E)-4-(benzyloxy)-3-((tert-butoxycarbonyl)amino) nonadec-1-en-1-yl)tetrahydro-2H-pyran-2-yl)methyl 3-phenylpropanoate (300 mg, 0.29 mmol, 1.0 equiv), MeOH (15 mL) and Pd(OH)₂ (30 mg) was stirred under an atmosphere of H₂ (balloon) for 16 h. The mixture was filtered through a pad of celite and the filtrate was concentrated under vacuum to give the product (100 mg, 51%) as a solid. m/z=mass calcd. for C₃₉H₆₇NO₉:693, found: 694 [M+H]⁺.

### Step 7: Synthesis of ((2R,3R,4R,5R,6R)-6-((3S,4R)-3-amino-4-hydroxynonadecyl)-3,4,5-trihydroxytetrahydro-2H-pyran-2-yl)methyl 3-phenylpropanoate

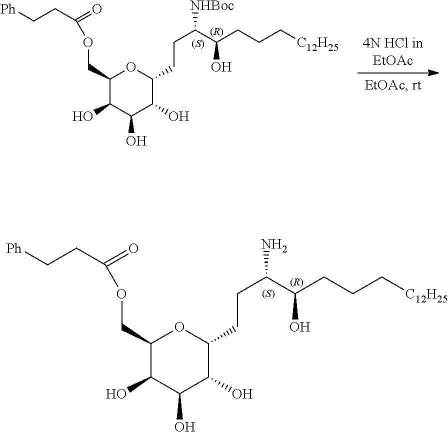

To a mixture of ((2R,3R,4R,5R,6R)-6-((3S,4R)-3-((tert-butoxycarbonyl)amino)-4-hydroxynonadecyl)-3,4,5-trihydroxytetrahydro-2H-pyran-2-yl)methyl 3-phenylpropanoate (100 mg, 0.14 mmol, 1.0 equiv) and EtOAc (4 mL) at rt was added HCl, 4N in EtOAc (4 mL). The mixture was stirred at rt for 2 h, then concentrated under vacuum to give the product (50 mg, 58%) as a solid. m/z=mass calcd. for C₃₄H₅₉NO₇:593, found: 594 [M+H]⁺.

### Step 8: Synthesis of ((2R,3R,4R,5R,6R)-6-((3S,4R)-3-(24-(3-fluorobicyclo[1.1.1]pentan-1-yl)tetracosanamido)-4-hydroxynonadecyl)-3,4,5-trihydroxytetrahydro-2H-pyran-2-yl)methyl 3-phenylpropanoate (Compound I-4)

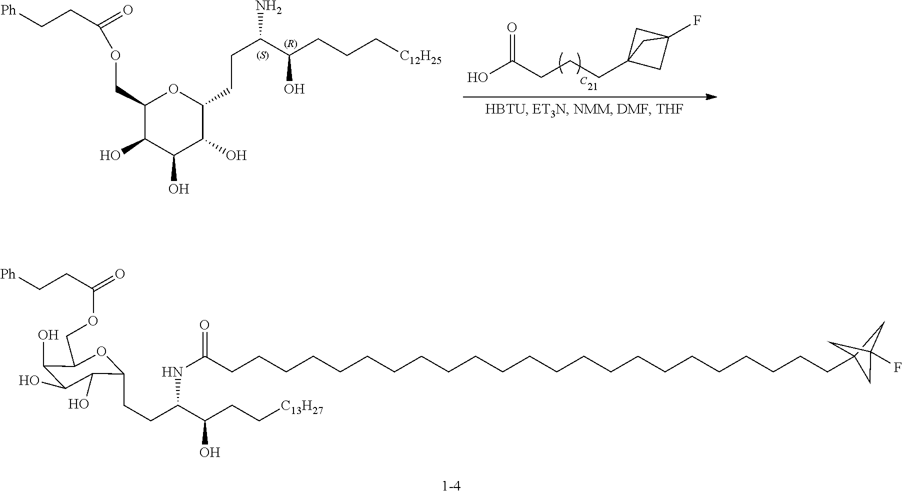

To a mixture of 24-(3-fluorobicyclo[1.1.1]pentan-1-yl)tetracosanoic acid (38 mg, 0.084 mmol, 1.0 equiv) and ((2R,3R,4R,5R,6R)-6-((3S,4R)-3-amino-4-hydroxynonadecyl)-3,4,5-trihydroxytetrahydro-2H-pyran-2-yl)methyl 3-phenylpropanoate (50 mg, 0.084 mmol, 1.0 equiv) in DMF (3 mL) and THF (3 mL) at rt under an atmosphere of N₂ were added HBTU (48 mg, 0.126 mmol, 1.5 equiv), Et₃N(0.1 mL), and NMM (0.1 mL). The mixture was stirred at rt for 16 h, then diluted with H₂O and extracted with EtOAc. The organic layer was concentrated under vacuum and the residue was purified by column chromatography on silica gel eluting with DCM/MeOH (9:1), then purified further by reverse-phase HPLC to give the product (7.3 mg, 8%) of the product as a solid. m/z=mass calcd. for C₆₃H₁₁₀FNO₈: 1027.82, found: 1028.95 [M+H]⁺; 1H NMR (400 MHZ, DMSO-d₆) δ 7.42 (d, J=8.9 Hz, 1H), 7.20-7.25 (m, 5H), 4.72-4.81 (m, 1H), 4.68 (dd, J=8.0, 5.3 Hz, 2H), 4.52 (d, J=7.5 Hz, 1H), 4.41 (d, J=6.2 Hz, 1H), 4.01 (s, 1H), 3.61-3.81 (m, 3H), 3.49-3.60 (m, 3H), 3.21 (t, J=7.8 Hz, 1H), 2.82-2.91 (m, 2H), 2.57 -2.67 (m, 2H), 2.01-2.10 (m, 2H), 1.89 (d, J=2.8 Hz, 5H), 1.30-1.35 (m, 76H), 0.82-0.89 (m, 3H).

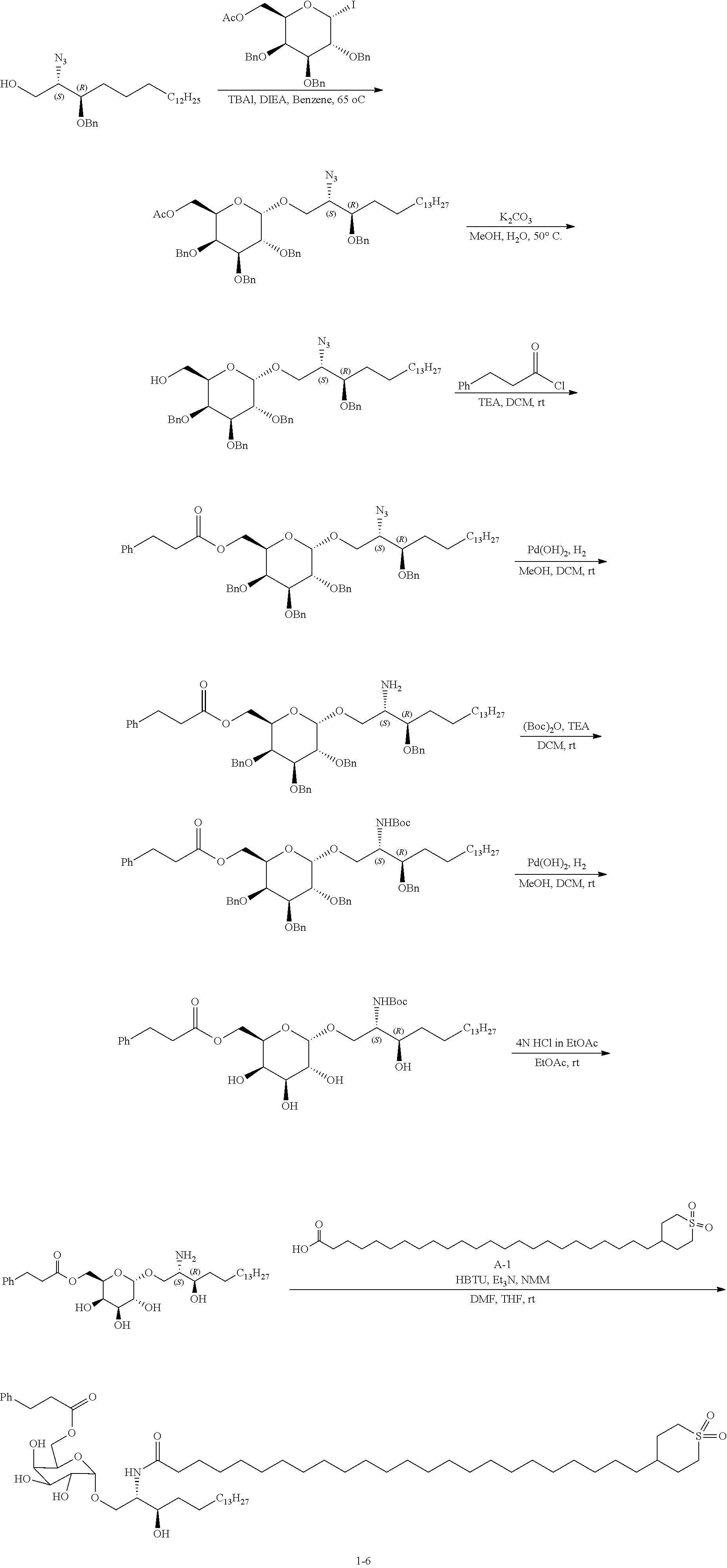

### Step 1: Synthesis of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl)methyl acetate

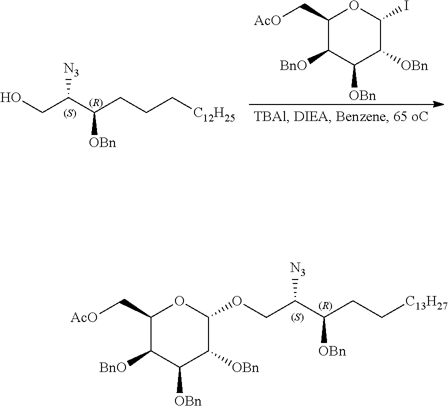

To a mixture of (2S,3R)-2-azido-3-(benzyloxy) octadecan-1-ol (700 mg, 1.68 mmol, 1.0 equiv) in benzene (20 mL) under an atmosphere of N₂ was added TBAI (55.57 g, 15.1 mmol, 9.0 equiv) and DIPEA (6.5 g, 5.0 mmol, 3.0 equiv). The mixture was heated to 65° C. and stirred for 0.5 h, then ((2R,3S,4S,5R,6R)-3,4,5-tris(benzyloxy)-6-iodotetrahydro-2H-pyran-2-yl)methyl acetate (3.0 g, 5.0 mmol, 3.0 equiv) was added and the mixture was stirred at 65° C. overnight. After cooling, the mixture was diluted with H₂O (30 mL) and extracted with EtOAc (3×30 mL). The combined organic layers were washed with brine (50 mL), dried over anhydrous Na₂SO₄ and concentrated under vacuum. The residue was purified by column chromatography on silica gel elututing with EtOAc/petroleum ether (1:3) to afford the product (1.3 g, 87%) as an oil. m/z=mass calcd. for C₅₄H₇₃N₃O₈:892, found: 915 [M+Na]⁺.

### Step 2: Synthesis of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl) methanol

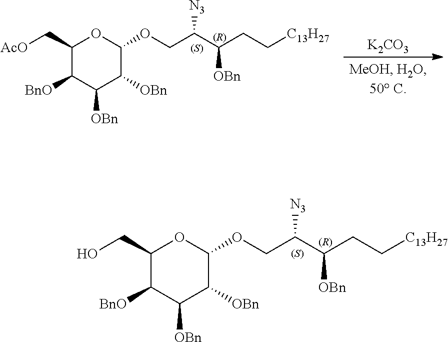

A mixture of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl)methyl acetate (1.3 g, 1.46 mmol, 1.0 equiv), K₂CO₃ (11.8 g, 13.1 mmol, 9.0 equiv), MeOH (15 mL) and H₂O (5 mL) was stirred at 50° C. overnight. H₂O (50 mL) was added and the mixture was extracted with EtOAc (3×20 mL). The combined organic layers were washed with brine (50 mL), dried over anhydrous Na₂SO₄ and concentrated under vacuum. The residue was purified by column chromatography on silica gel eluting with EtOAc/petroleum ether (1:2) to give the product (750 mg, 60%) as an oil. m/z=mass calcd. for C₅₂H₇₁N₃O₇:850, found: 873 [M+Na]⁺.

### Step 3: Synthesis of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl)methyl 3-phenylpropanoate

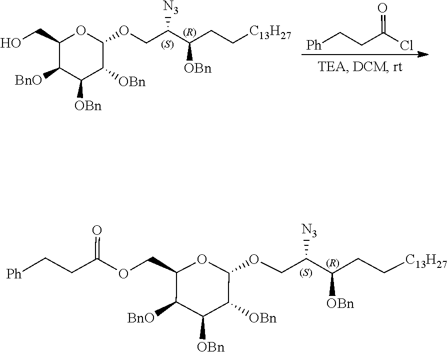

To a mixture of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl) methanol (750 mg, 0.88 mmol, 1.0 equiv), Et₃N (357 mg, 3.53 mmol, 4.0 equiv) and DCM (10 mL) at rt under an atmosphere of N₂ was added benzenepropanoyl chloride (446 mg, 2.65 mmol, 3.0 equiv). The mixture was stirred at rt overnight, then diluted with H₂O (30 mL) and extracted with EtOAc (3×30 mL). The combined organic layers were washed with brine (30 mL), dried over anhydrous Na₂SO₄ and concentrated under vacuum. The residue was purified by column chromatography on silica gel eluting with EtOAc/petroleum ether (1:3) to give the product (700 mg, 81%) as an oil. m/z=mass calcd. for C₆₁H₇₉N₃O₈:982, found: 1005 [M+Na]⁺.

### Step 4: Synthesis of ((2R,3S,4S,5R,6S)-6-(((2S,3R)-2-amino-3-(benzyloxy) octadecyl)oxy)-3,4,5-tris(benzyloxy) tetrahydro-2H-pyran-2-yl)methyl 3-phenylpropanoate

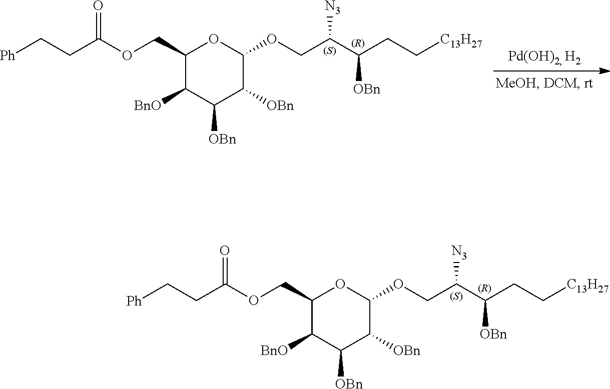

A mixture of ((2R,3S,4S,5R,6S)-6-(((2S,3R)-2-azido-3-(benzyloxy) octadecyl)oxy)-3,4,5-tris(benzyloxy) tetrahydro-2H-pyran-2-yl)methyl 3-phenylpropanoate (600 mg, 0.61 mmol, 1.0 equiv), MeOH (15 mL), DCM (3 mL) and Pd(OH)₂ (60 mg) was stirred under an atmosphere of H₂ (balloon) for 1 h. The mixture was filtered through a pad of celite and the filtrate was concentrated under vacuum to give the product (400 mg, 68%) as a solid. m/z=mass calcd. for C₆₁H₈₁NO₈:956, found: 979 [M+Na]⁺.

### Step 5: Synthesis of ((2R,3S,4S,5R,6S)-3,4,5-tris(benzyloxy)-6-(((2S,3R)-3-(benzyloxy)-2-((tert-butoxycarbonyl)amino) octadecyl)oxy)tetrahydro-2H-pyran-2-yl)methyl 3-phenylpropanoate

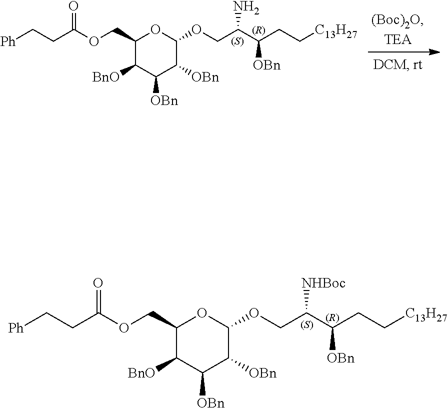

To a mixture of ((2R,3S,4S,5R,6S)-6-(((2S,3R)-2-amino-3-(benzyloxy) octadecyl)oxy)-3,4,5-tris(benzyloxy) tetrahydro-2H-pyran-2-yl)methyl 3-phenylpropanoate (360 mg, 0.38 mmol, 1.0 equiv), Et₃N(46 mg, 0.45 mmol, 1.2 equiv) and DCM (3 mL) was added (Boc) 20 (87 mg, 0.4 mmol, 1.05 equiv). The mixture was stirred at rt for 3 h, then diluted with H₂O (10 mL) and extracted with EtOAc (3×20 mL). The combined organic layers were washed with brine (20 mL), dried over anhydrous Na₂SO₄ and concentrated under vacuum. The residue was purified by column chromatography on silica gel eluting with EtOAc/petroleum ether (1:3) to give the product (350 mg, 88%) as a solid. m/z=mass calcd. for C₆₆H₈₉NO₁₀:1056, found: 1079 [M+Na]⁺.

### Step 6: Synthesis of ((2R,3R,4S,5R,6S)-6-(((2S,3R)-2-((tert-butoxycarbonyl)amino)-3-hydroxyoctadecyl)oxy)-3,4,5-trihydroxytetrahydro-2/-pyran-2-yl)methyl 3-phenylpropanoate

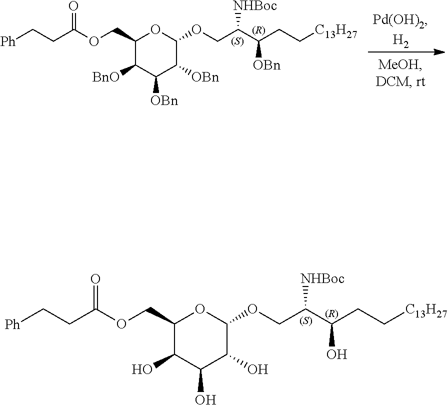

A mixture of ((2R,3S,4S,5R,6S)-3,4,5-tris(benzyloxy)-6-(((2S,3R)-3-(benzyloxy)-2-((tert-butoxycarbonyl)amino) octadecyl)oxy)tetrahydro-2H-pyran-2-yl)methyl 3-phenylpropanoate (350 mg, 0.33 mmol, 1.0 equiv), MeOH (10 mL), DCM (2 mL) and Pd(OH)₂ (35 mg) was stirred under an atmosphere of H₂ (balloon) for 16 h. The mixture was filtered and the filter cake was washed with MeOH. The filtrate was concentrated under vacuum to give the product (200 mg, 87%) as a solid. m/z=mass calcd. for C₃₈H₆₅NO₁₀:695, found: 696 [M+H]⁺.

### Step 7: Synthesis of ((2R,3R,4S,5R,6S)-6-(((2S,3R)-2-amino-3-hydroxyoctadecyl)oxy)-3,4,5-trihydroxytetrahydro-2H-pyran-2-yl)methyl 3-phenylpropanoate

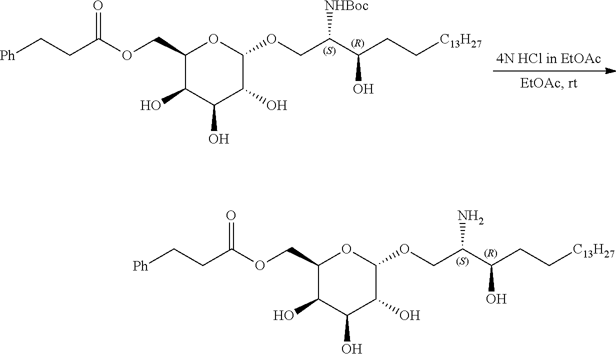

To a mixture of ((2R,3R,4S,5R,6S)-6-(((2S,3R)-2-((tert-butoxycarbonyl)amino)-3-hydroxyoctadecyl)oxy)-3,4,5-trihydroxytetrahydro-2H-pyran-2-yl)methyl 3-phenylpropanoate (200 mg, 0.29 mmol, 1.0 equiv) in EtOAc (3 mL) at rt was added 4N HCl in EtOAc (3 mL). The mixture was stirred at rt for 1 h, then concentrated under vacuum to give the product (120 mg, 70%) as a solid. m/z=mass calcd. for C₃₈H₆₅NO₁₀:595, found: 596 [M+H]⁺.

### Step 8: Synthesis of ((2R,3R,4S,5R,6S)-6-(((2S,3R)-2-(24-(1,1-dioxidotetrahydro-2H-thiopyran-4-yl)tetracosanamido)-3-hydroxyoctadecyl)oxy)-3,4,5-trihydroxytetrahydro-2H-pyran-2-yl)methyl 3-phenylpropanoate (Compound I-6)

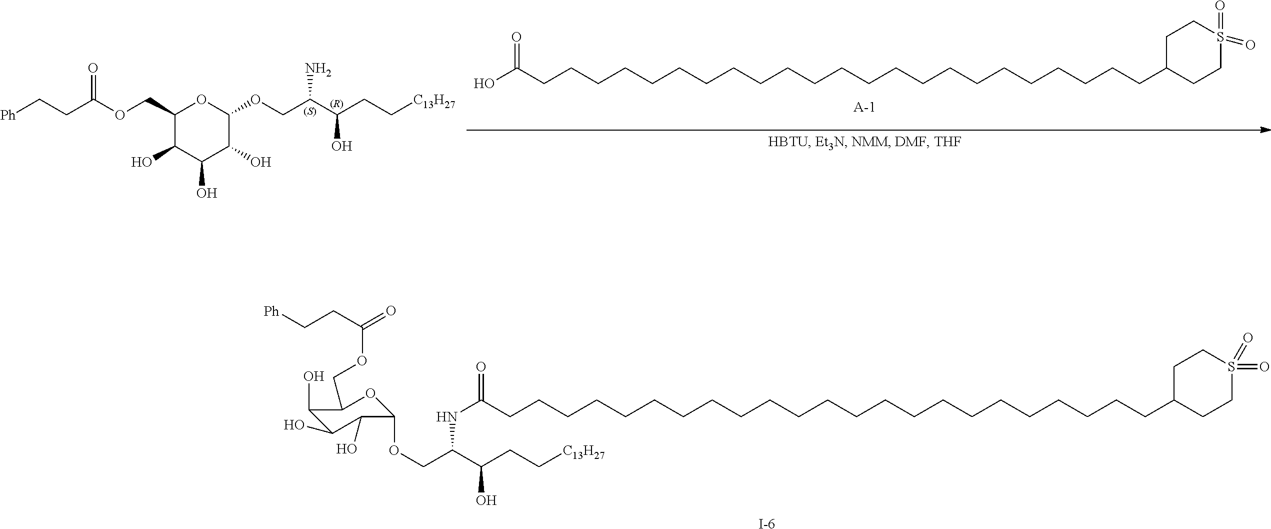

To a mixture of 24-(1,1-dioxidotetrahydro-2H-thiopyran-4-yl)tetracosanoic acid (Intermediate A-1, 59 mg, 0.117 mmol, 1.0 equiv) and ((2R,3R,4S,5R,6S)-6-(((2S,3R)-2-amino-3-hydroxyoctadecyl)oxy)-3,4,5-trihydroxytetrahydro-2H-pyran-2-yl)methyl 3-phenylpropanoate (70 mg, 0.117 mmol, 1.0 equiv) in DMF (3 mL) and THF (3 mL) at rt under an atmosphere of N₂ were added HBTU (67 mg, 0.18 mmol, 1.5 equiv), Et₃N(0.1 mL), and NMM (0.1 mL). The mixture was stirred at rt for 16 h, then diluted with H₂O (10 mL) and extracted with EtOAc (30 mL×3). The combined organic layers were concentrated under vacuum and the residue was purified by column chromatography on silica gel eluting with DCM/MeOH (9:1), then additional purification using reverse-phase HPLC to give the product (21.2 mg, 16%). m/z=mass calcd. for C₆₂H₁₁₁NO₁₁S: 1077.79, found: 1078.60 [M+H]⁺; 1H NMR (400 MHZ, DMSO-d₆) δ 7.56 (d, J=9.0 Hz, 1H), 7.12-7.33 (m, 5H), 4.50-4.70 (m, 4H), 4.22 (d, J=7.8 Hz, 1H), 3.99-4.11 (m, 2H), 3.70-3.88 (m, 3H), 3.65 (s, 1H), 3.54 (d, J=4.0 Hz, 4H), 3.02-3.17 (m, 2H), 2.98 (s, 2H), 2.83 (s, 2H), 2.60 (d, J=8.0 Hz, 2H), 1.90-2.12 (m, 4H), 1.43-1.50 (m, 8H), 1.20-1.25 (m, 67H), 0.85 (t, J=6.7 Hz, 3H).

### Example A7: Compound I-7

### Step 1: Synthesis of N-((3S,4S,5R)-4,5-dihydroxy-1-((2R,3R,4R,5R,6R)-3,4,5-trihydroxy-6-(hydroxymethyl)tetrahydro-2H-pyran-2-yl) nonadecan-3-yl)-24-(3-fluorobicyclo[1.1.1]pentan-1-yl)tetracosanamide (Compound I-7)

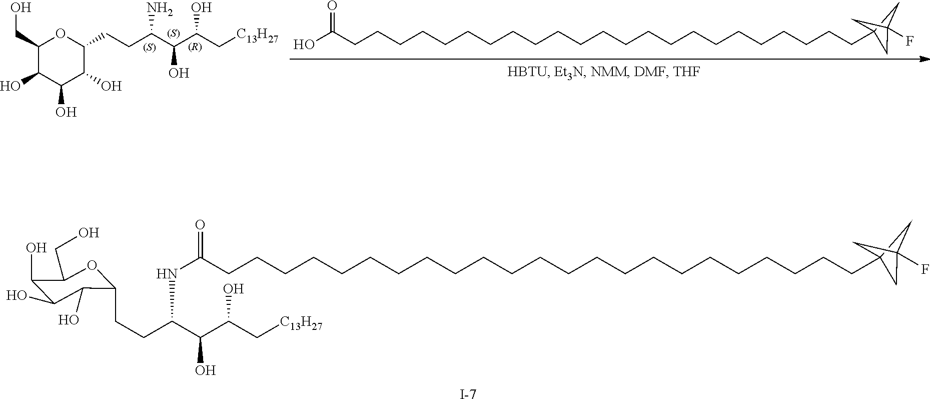

To a mixture of 24-(3-fluorobicyclo[1.1.1]pentan-1-yl)tetracosanoic acid (75 mg, 0.17 mmol, 1.0 equiv) and (2R,3R,4R,5R,6R)-2-((3S,4S,5R)-3-amino-4,5-dihydroxynonadecyl)-6-(hydroxymethyl)tetrahydro-2H-pyran-3,4,5-triol (80 mg, 0.17 mmol, 1.0 equiv) in DMF (3 mL) and THF (3 mL) at rt under an atmosphere of N₂ were added HBTU (95 mg, 0.25 mmol, 1.5 equiv), Et₃N(0.1 mL), and NMM (0.1 mL). The mixture was stirred at rt for 16 h, then diluted with H₂O and extracted with EtOAc. The organic layer was concentrated under vacuum and the residue was purified by column chromatography on silica gel eluting with DCM/MeOH (9:1). The resulting solid was washed with CH 3CN and dried to give the product (7.8 mg, 4%) as a solid. m/z=mass calcd. for C₅₄H₁₀₂FNO₈:911.76, found: 912.70 [M+H]⁺; 1H NMR (400 MHZ, DMSO-d₆) δ 7.44 (d, J=9.2 Hz, 1H), 4.61 (d, J=3.9 Hz, 1H), 4.47-4.57 (m, 2H), 4.39 (s, 1H), 4.30 (d, J=4.8 Hz, 1H), 4.14 (d, J=6.8 Hz, 1H), 3.76 (d, J=26.9 Hz, 2H), 3.62 (s, 2H), 3.49 (d, J=5.4 Hz, 2H), 3.42 (d, J=5.4 Hz, 2H), 3.23 (s, 1H), 2.18 (t, J=7.4 Hz, 1H), 2.03 (q, J=12.9, 10.1 Hz, 1H), 1.87 (d, J=2.8 Hz, 6H), 1.72 (d, J=13.5 Hz, 1H), 1.58 (d, J=7.8 Hz, 1H), 1.33-1.50 (m, 7H), 1.31-1.08 (m, 66H), 0.85 (t, J=6.5 Hz, 3H).

### Example A8: Compound I-8

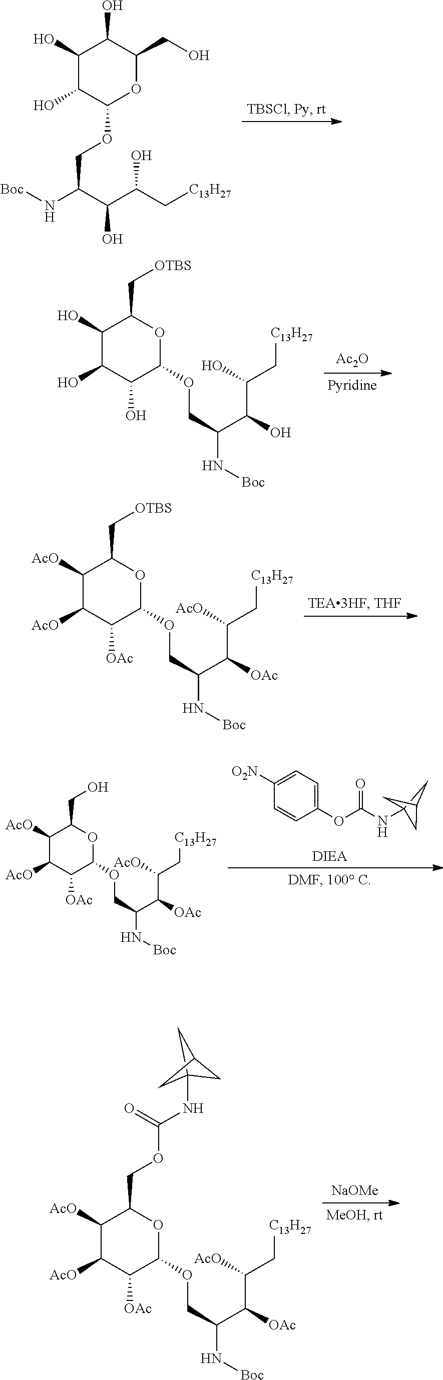

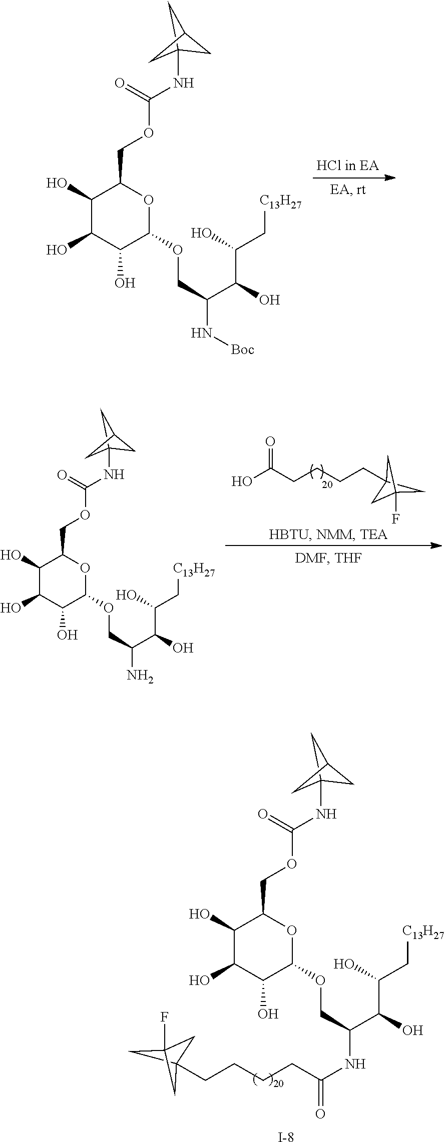

### Step 1: Synthesis of tert-butyl ((2S,3S,4R)-1-(((2S,3R,4S,5R,6R)-6-(((tert-butyldimethylsilyl)oxy)methyl)-3,4,5-trihydroxytetrahydro-2H-pyran-2-yl)oxy)-3,4-dihydroxyoctadecan-2-yl) carbamate

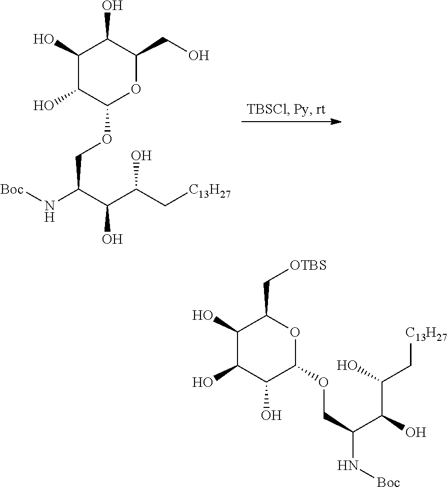

To a mixture of tert-butyl ((2S,3S,4R)-3,4-dihydroxy-1-(((2S,3R,4S,5R,6R)-3,4,5-trihydroxy-6-(hydroxymethyl)tetrahydro-2H-pyran-2-yl)oxy) octadecan-2-yl) carbamate (700 mg, 1.2 mmol, 1.0 equiv) in pyridine (10 mL) at rt was added tert-butyldimethylchlorosilane (546 mg, 3.6 mmol, 3.0 equiv) in portions. The mixture was stirred at rt for 3 h, then diluted with H₂O (10 mL) and extracted with EtOAc (50 mL×3). The combined organic layers were washed with brine (20 mL), dried over anhydrous Na₂SO₄ and concentrated under vacuum. The residue was purified by column chromatography on silica gel eluting with MeOH/DCM (5:95 to 15:85) to give the product (600 mg, 71%) as an oil. m/z=mass calcd. for C₃₅H₇₁NO₁₀Si: 693.48, found 594.35 [M-Boc+H]⁺.

### Step 2: Synthesis of (2R,3S,4S,5R,6S)-2-(((tert-butyldimethylsilyl)oxy)methyl)-6-(((2S,3S,4R)-3,4-diacetoxy-2-((tert-butoxycarbonyl)amino) octadecyl)oxy)tetrahydro-2H-pyran-3,4,5-triyl triacetate

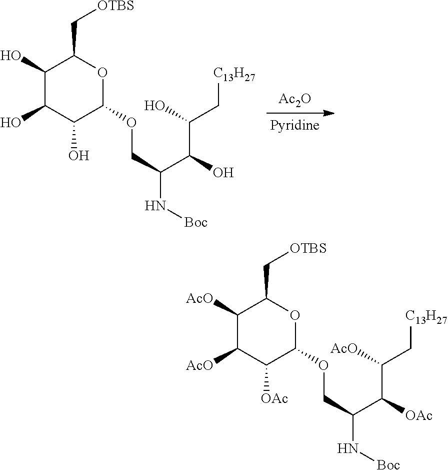

To a mixture of tert-butyl ((2S,3S,4R)-1-(((2S,3R,4S,5R,6R)-6-(((tert-butyldimethylsilyl)oxy)methyl)-3,4,5-trihydroxytetrahydro-2H-pyran-2-yl)oxy)-3,4-dihydroxyoctadecan-2-yl) carbamate (600 mg, 0.87 mmol, 1.0 equiv) in pyridine (4 mL) at rt was added Ac₂O (4 mL) dropwise. The mixture was stirred at rt for 16 h, then diluted with H₂O (20 mL) and extracted with EtOAc (20 mL×3). The combined organic layers were washed with brine (20 mL), dried over anhydrous Na₂SO₄ and concentrated under vacuum. The residue was purified by column chromatography on silica gel eluting with EtOAc/petroleum ether (15:85 to 1:4) to give the product (700 mg, 89%) as an oil. m/z=mass calcd. for C₄₅H₈₁NO₁₅Si: 903.54, found 804.40 [M-Boc+H]⁺.

Step 3: Synthesis of (2S,3R,4S,5S,6R)-2-(((2S,3S,4R)-3,4-diacetoxy-2-((tert-butoxycarbonyl)amino) octadecyl)oxy)-6-(hydroxymethyl)tetrahydro-2H-pyran-3,4,5-triyl triacetate

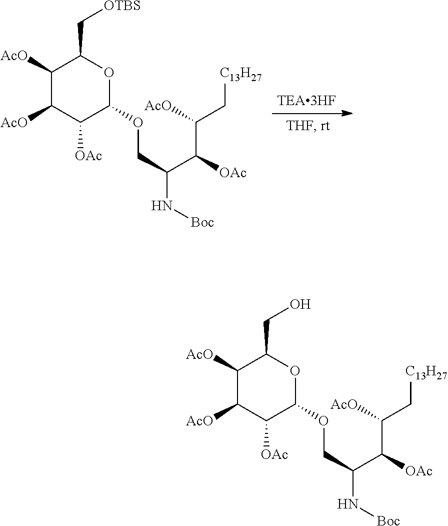

A mixture of (2R,3S,4S,5R,6S)-2-(((tert-butyldimethylsilyl)oxy)methyl)-6-(((2S,3S,4R)-3,4-diacetoxy-2-((tert-butoxycarbonyl)amino) octadecyl)oxy)tetrahydro-2H-pyran-3,4,5-triyl triacetate (500 mg, 0.55 mmol, 1.0 equiv) and Et₃N 3HF [triethylamine trihydrochloride] (2 mL) in THF (6 mL) was stirred at rt for 4 h. The mixture was diluted with 1M HCl (10 mL) and extracted with EtOAc (25 mL×3). The combined organic layers were washed with brine (20 mL), dried over anhydrous Na₂SO₄ and concentrated under vacuum. The residue was purified by column chromatography on silica gel eluting with EtOAc/petroleum ether (1:1 to 55:45) to give the product (400 mg, 92%) as an oil. m/z=mass calcd. for C₃₉H₆₇NO₁₅:789.45, found 690.25 [M-Boc+H]⁺.

### Step 4: Synthesis of (2R,3S,4S,5R,6S)-2-(((bicyclo[1.1.1]pentan-1-ylcarbamoyl)oxy)methyl)-6-(((2S,3S,4R)-3,4-diacetoxy-2-((tert-butoxycarbonyl)amino) octadecyl)oxy)tetrahydro-2H-pyran-3,4,5-triyl triacetate

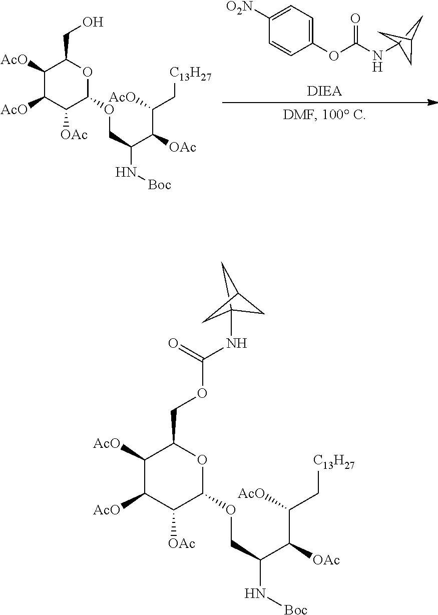

A mixture of (2S,3R,4S,5S,6R)-2-(((2S,3S,4R)-3,4-diacetoxy-2-((tert-butoxycarbonyl)amino) octadecyl)oxy)-6-(hydroxymethyl)tetrahydro-2H-pyran-3,4,5-triyl triacetate (100 mg, 0.13 mmol, 1.0 equiv), 4-nitrophenyl bicyclo[1.1.1]pentan-1-ylcarbamate (94 mg, 0.38 mmol, 3.0 equiv) and DIPEA (98 mg, 0.76 mmol, 6.0 equiv) in DMF (3 mL) was stirred at 100° C. under an atmosphere of N₂ overnight. The mixture was diluted with H₂O (10 Ml) and extracted with EtOAc (25 mL×3). The combined organic layers were washed with brine (10 mL), dried over anhydrous Na₂SO₄ and concentrated under vacuum. The residue was purified by column chromatography on silica gel eluting with EtOAc/petroleum ether (1:5 to 1:4) to give the product (80 mg, 70%) as an oil. m/z=mass calcd. for C₄₅H₇₄N₂O₁₆:898.50, found 799.35 [M-Boc+H]⁺.

### Step 5: Synthesis of ((2R,3R,4S,5R,6S)-6-(((2S,3S,4R)-2-((tert-butoxycarbonyl)amino)-3,4-dihydroxyoctadecyl)oxy)-3,4,5-trihydroxytetrahydro-2H-pyran-2-yl)methyl bicyclo[1.1.1]pentan-1-ylcarbamate

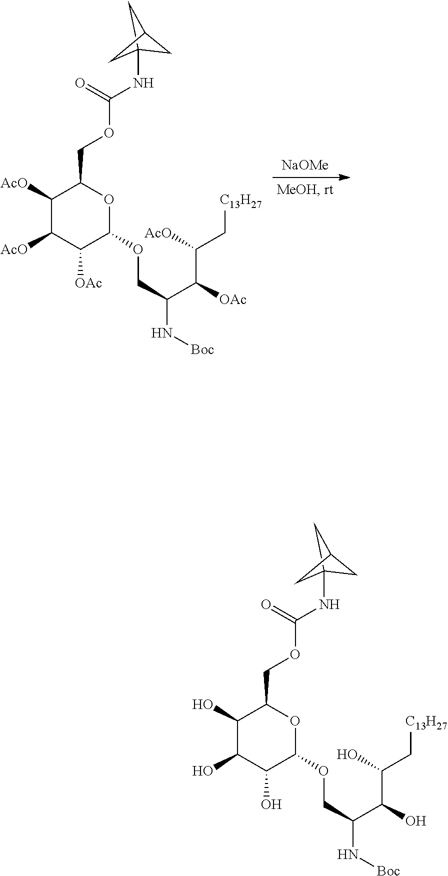

A mixture of (2R,3S,4S,5R,6S)-2-(((bicyclo[1.1.1]pentan-1-ylcarbamoyl)oxy)methyl)-6-(((2S,3S,4R)-3,4-diacetoxy-2-((tert-butoxycarbonyl)amino) octadecyl)oxy)tetrahydro-2H-pyran-3,4,5-triyl triacetate (80 mg, 0.09 mmol, 1.0 equiv) and NaOMe (1 mg, 0.018 mmol, 0.2 equiv) in MeOH (2 mL) at rt was stirred for 2 h. The mixture was concentrated under vacuum and the residue was purified by column chromatography on silica gel eluting with MeOH/DCM (1:9 to 15:85) to give the product (45 mg, 73%) as an oil. m/z=mass calcd. for C₃₅H₆₄N₂O₁₁:688.45, found 589.30 [M-Boc+H]⁺.

### Step 6: Synthesis of ((2R,3R,4S,5R,6S)-6-(((2S,3S,4R)-2-amino-3,4-dihydroxyoctadecyl)oxy)-3,4,5-trihydroxytetrahydro-2H-pyran-2-yl)methyl bicyclo[1.1.1]pentan-1-ylcarbamate

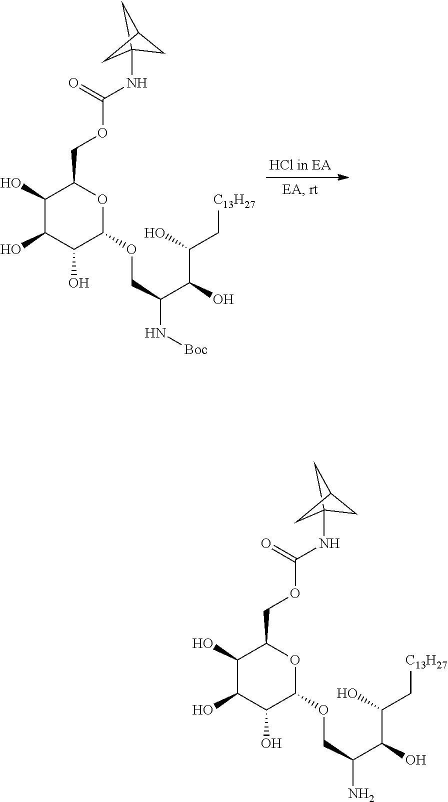

A mixture of ((2R,3R,4S,5R,6S)-6-(((2S,3S,4R)-2-((tert-butoxycarbonyl)amino)-3,4-dihydroxyoctadecyl)oxy)-3,4,5-trihydroxytetrahydro-2H-pyran-2-yl)methyl bicyclo[1.1.1]pentan-1-ylcarbamate (45 mg, 0.065 mmol, 1.0 equiv) and HCl in EtOAc (1 mL) was stirred at rt for 2 h. The mixture was concentrated under reduced pressure to give the product (35 mg, 91%) as a solid. m/z=mass calcd. for C₃₀H₅₆N₂O₉:588.40, found 589.30 [M+H]⁺.

### Step 7: Synthesis of ((2R,3R,4S,5R,6S)-6-(((2S,3S,4R)-2-(24-(3-fluorobicyclo[1.1.1]pentan-1-yl)tetracosanamido)-3,4-dihydroxyoctadecyl)oxy)-3,4,5-trihydroxytetrahydro-2H-pyran-2-yl)methylbicyclo[1.1.1]pentan-1-ylcarbamate (Compound I-8)

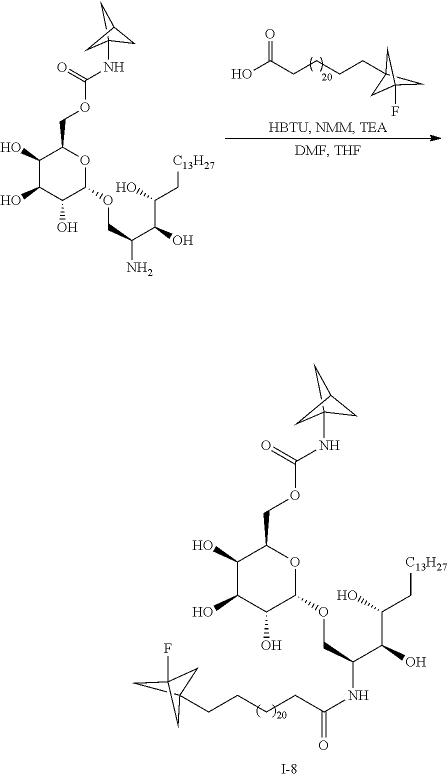

A mixture of 24-(3-fluorobicyclo[1.1.1]pentan-1-yl)tetracosanoic acid (35 mg, 0.076 mmol, 1.5 equiv), HBTU (19 mg, 0.076 mmol, 1.5 equiv), Et₃N(15 mg, 0.15 mmol, 3.0 equiv), NMM (15 mg, 0.15 mmol, 3.0 equiv) and ((2R,3R,4S,5R,6S)-6-(((2S,3S,4R)-2-amino-3,4-dihydroxyoctadecyl)oxy)-3,4,5-trihydroxytetrahydro-2H-pyran-2-yl)methyl bicyclo[1.1.1]pentan-1-ylcarbamate (30 mg, 0.051 mmol, 1.0 equiv) in DMF (1.5 mL) and the (1.5 mL) was stirred at rt overnight. The mixture was diluted with H₂O (3 mL) and extracted with EtOAc (20 mL). The combined organic layers were washed with brine (5 mL), dried over anhydrous Na₂SO₄ and concentrated under vacuum. The crude residue was purified by column chromatography on silica gel eluting with DCM/MeOH to give a solid, and the resulting solid was washed with CH₃CN to give the product (15.2 mg, 29%) as a solid. m/z=mass calcd. for C₅₉H₁₀₇FN₂O₁₀: 1022.79, found: 1023.80 [M+H]⁺; 1H NMR (300 MHz, DMSO-d₆) δ 7.30-8.04 (m, 2H), 4.54-4.73 (m, 4H), 4.20-4.38 (m, 2H), 3.78-3.96 (m, 4H), 3.48-3.58 (m, 6H), 2.34 (s, 1H), 1.99-2.10 (m, 2H), 1.88 (d, J=14.5 Hz, 12H), 1.36-1.60 (m, 8H), 1.12-1.28 (m, 62H), 0.85 (t, J=6.7 Hz, 3H).

### B. Methods for Assessing Compound Activity

### Example B1: In vitro Activation of Human iTCR through a Jurkat Reporter Cell Line

To determine the human iTCR activation potential induced by the compounds described herein, a Jurkat cell line (JiNKT) was transfected with the human iTCR, and GFP under the NFKB promoter (cell line licensed from the Medical University of Vienna). A BWS147 cell line (BWSTIM) was also transfected with CD80 and CD1d to act as the antigen-presenting cell.

Any of the compounds provided herein may be used in this example, including the exemplary compounds of Tables 1 and 2. Test compounds were dissolved in DMSO at a 5 mg/mL stock solution. BWSTIM cells were loaded with test compounds at varying concentrations for 4 hours at 37° C. at a concentration of 20k cells/well in 200 μL of media in a u-bottom 96 well dish. BWSTIM cells were washed 2× with media, then incubated with JiNKT cells at a concentration of 80k cells/well in a u-bottom 96 well dish. Cells were co-cultured for 18 to 24 hours. The percentage of cells expressing high levels of GFP was measured through flow cytometry (after gating out the mCD45+ BWSTIM cells). EC50s for several exemplary compounds are shown in Table B1 below.

TABLE B1

| Cmpd. No. | Human In Vitro EC50 (μg/mL) |
|---|---|
| I-2 | 1.60 |
| I-3 | 0.95 |
| I-4 | 0.6391 |
| I-6 | 0.86 |
| I-7 | 1.08 |

### Example B2: In vitro Activation of Mouse iTCR through a DN3-a4 1.2 INKT Hybridoma Cell Line

To determine the mouse iTCR activation potential induced by the compounds described herein, a mouse iNKT hybridoma cell line (DN3.2) from the La Jolla Institute for Allergy and Immunology is used as the iNKT. The BWS147 cell line (BWSTIM) with CD80 and CD1d act as the antigen-presenting cell.

Any of the compounds provided herein may be used in this example, including the exemplary compounds of Tables 1 and 2. Test compounds are dissolved in DMSO as a 5 mg/ml stock solution. BWSTIM cells are loaded with activators or α-GalCer of varying concentrations for 4 hours at 37° C. at a concentration of 20k cells/well in 200 μL of media in a u-bottom 96 well dish. BWSTIM cells are washed 2× with media, then incubated with DN3.2 cells at a concentration of 80k cells/well in a u-bottom 96 well dish. Cells are co-cultured for 48 hours. Media is collected and IL-2 is measured using the CisBio HTRF ELISA detection kit.

### Example B3: Cytokine Secretion

To determine the activation profile induced by the compounds described herein, primary human iNKT cells are co-cultured with drug-loaded BWSTIM cells. Any of the compounds provided herein may be used in this example, including the exemplary compounds of Tables 1 and 2.

Day 0: PBMCs are isolated from human blood using the STEMCELL TECHNOLOGIES™ SepMate™ PBMC isolation system. iNKT cells are then selected using the Miltenyi NKT magnetic cell separation kit.

Day 2: Test compounds are dissolved in DMSO at a 5 mg/mL stock solution. BWSTIM cells are fixed with mitomycin C, then loaded with 10 μg/mL of disclosed activator compounds or α-GalCer for 4 hours at 37° C. at a concentration of 20k cells/well in 200 μL of media in a u-bottom 96 well dish. Cells are co-cultured with 80k 6B11+ selected primary human iNKT cells.

D4: Media is collected. Cytokines are measured using the Sartorious 4Plex kit on the iQue3 cytometer.

### Example B4: In vivo IFNγ Activation and iNKT Cell Expansion in C57BL/6J Mice

Any of the compounds provided herein may be used in this example, including the exemplary compounds of Tables 1 and 2. To determine the expansion induced by the compounds described herein, test compounds were injected into C57BL/6J mice. Serum IFNγ levels and the expansion of iNKT cells within splenocytes was measured 4 days post-IP injection.

Eight weeks old C57BL/6J mice were injected (I.P.) with 2 μg of test compounds. The test compounds were either dissolved in DMSO at a 5 mg/mL stock solution, or formulated into liposomes through thin-film rehydration, then extrusion through 200 nm filters. Liposome-based formulations were constructed using soy phosphatidylcholine, cholesterol, and test compound in a 2:1:0.15 ratio. Twenty hours post-injection, blood was collected from the tail to measure the level of serum IFNγ using an ELISA kit from Biolegend. Four days post-injection, the mice were sacrificed and the splenocytes were isolated. The percentage of iNKT cells within the spleens was measured using flow cytometry, selecting for live cells and mouse CD1d-α-GalCer tetramer+ cells.

In response to injection of several exemplary compounds, the average serum IFNγ collected and fold change of cells are shown in Table B4 below.

TABLE B4

| | | Naïve Mouse In Vivo |
|---|---|---|
| | Naïve Mouse In Vivo | CD3 + iNKT + cells (Fold |
| Cmpd. No. | IFNγ (pg/mL) | increase over veh control) |
| I-2 | 8128 ± 1209 | 2.21 ± 0.38 |
| I-3 | 8506 ± 790 | 1.12 ± 0.19 |
| I-4 | 28208 ± 4723 | 4.09 ± 0.78 |
| I-6 | 43930 ± 43396 | 1.89 ± 0.44 |

### Example B5: Diet Induced Obesity (HFD) Mouse Model Study

Senescence is a feature of pre-adipocytes in obese individuals. To study the efficacy of the compounds described herein in decreasing senescence in fat, a diet induced obesity high fat diet (HFD) mouse model is used. Any of the compounds provided herein may be used in this example, including the exemplary compounds of Tables 1 and 2.

22-week-old HFD mice are injected with α-GalCer as a control and compared with test compounds of the present disclosure. The blood and spleen (or adipose tissue) are collected to measure iNKT activation and expansion, respectively. Cells in the eWAT (adipose tissue) are also collected and measured for % senescent cells. HFD mice are compared to non-HFD (normal diet) mice.

22-week-old C57BL/6J mice on a chow or high-fat diet (HFD) are injected (I.P) with 2 μg of α-GalCer or test compounds. The test compounds are either dissolved in DMSO at a 5 mg/mL stock solution, or formulated into liposomes through thin-film rehydration, then extrusion through 200 nm filters. Liposome-based formulations are constructed using soy phosphatidylcholine, cholesterol, and an activator compound or α-GalCer in a 2:1:0.15 ratio. Two or twenty hours post-injection, blood is collected from the tail to measure the level of IFNγ using ELISA. Four days post-injection, mice are sacrificed to collect eWAT or spleen. Spleen is used to measure the number of iNKT cells, and eWAT is used to measure the number of iNKT cells and senescent cells using flow cytometry. iNKT cells are identified in the digested adipose tissue by gating live cells and mouse CD1d-α-GalCer tetramer+ cells. Senescent cells are measured within the processed adipose tissue by selecting mCD45-cells, and C12FDG^{HIGH} cells.

The expansion of immune cells in the spleen of: 1) mice on a normal diet; 2) mice on a high fat diet injected with diluent; 3) mice on a high fat diet injected with α-GalCer and 4) mice on a high fat diet injected with test compounds is compared.

The expansion of immune cells in the eWAT of: 1) mice on a normal diet; 2) mice on a high fat diet injected with diluent; 3) mice on a high fat diet injected with α-GalCer and 4) mice on a high fat diet injected with test compounds is compared.

Non-immune C12FDG+ cells from eWAT are identified via flow cytometry. A decrease in the number of C12FDG positive cells indicates a decrease in the number of senescent cells in eWAT.

### Example B6: Inactivity of Compounds in In vitro Studies with Human and Mouse iTCR in Jurkat Reporter and DN3-a4 1.2 INKT Hybridoma Cell Lines

A Jurkat cell line (JiNKT) is transfected with the human iTCR, and GFP under the NFKB promoter. A BWS147 cell line (BWSTIM) is also transfected with CD80 and CD1d to act as the antigen-presenting cell. A mouse iNKT hybridoma cell line (DN3.2) is used as the iNKT. The BWS147 cell line (BWSTIM) with CD80 and CD1d acts as the antigen-presenting cell.

Any of the compounds provided herein may be used in this example, including the exemplary compounds of Tables 1 and 2.

### GFP Expresion Studies

Test compounds are dissolved in DMSO at a 5 mg/mL stock solution. BWSTIM cells are loaded with test compounds for 4 hours at 37° C. at a concentration of 20k cells/well in 200 μL of media in a u-bottom 96 well dish. BWSTIM cells are washed 2× with media, then incubated with JiNKT cells at a concentration of 80k cells/well in a u-bottom 96 well dish. Cells are co-cultured for 24 hours. The percentage of cells expressing high levels of GFP is measured through flow cytometry. 1 μg/mL of molecule is incubated with BWSTIM+JiNKT.

### IL-2 Expression Studies

Test compounds are dissolved in DMSO at a 5 mg/mL stock solution. BWSTIM cells are loaded with compounds for 4 hours at 37° C. at a concentration of 20k cells/well in 200 μL of media in a u-bottom 96 well dish. BWSTIM cells are washed 2× with media, then incubated with DN3.2 cells at a concentration of 80k cells/well in a u-bottom 96 well dish. Cells are co-cultured for 48 hours. Media is collected and IL-2 is measured using the CisBio HTRF ELISA detection kit.

### Cytokine Secretion Studies

To determine the activation profile induced by the compounds described herein, primary human iNKT cells are co-cultured with drug-loaded BWSTIM cells. Test compounds are dissolved in DMSO at a 5 mg/mL stock solution. BWSTIM cells re loaded with compounds for 4 hours at 37° C. at a concentration of 20k cells/well in 200 μL of media in a u-bottom 96 well dish. Cells are co-cultured with 100k 6B11+ selected primary human iNKT cells. Media is collected 2 days later. Cytokines are measured using the Sartorious 4Plex kit on the iQue3 cytometer.

The secretion of the cytokine interferon gamma (IFNγ), tumor necrosis factor alpha (TNFα), interleukin-4 (IL-4) and interleukin-6 (IL-6) in response to incubation with test compounds is measured. The secretion is compared to secretion by cells in the absence of drug loading as a negative control.

### Example B7: Activation of INKT Cells Selectively Kills Senescent Cells in vitro

This example explores selective reduction in the presence of senescent cells by iNKT mediates killing in an in vitro sample. Human iNKT cells are isolated and activated by incubation with test compounds, with or without being first loaded onto an antigen presenting cell. Any of the compounds provided herein may be used in this example, including the exemplary compounds of Tables 1 and 2.

Activated iNKT cells are combined with samples containing healthy cells and senescent cells. The presence of senescent cells over time as compraed to non-senescent cells over time is monitored.

### Example B8: Activation of INKT in an in vivo IPF model

This example explores activation of iNKT with a test compound in an in vivo rodent model of idiopathic pulmonary fibrosis. Any of the compounds provided herein may be used in this example, including the exemplary compounds of Tables 1 and 2. An observed decrease in senescent cells and a reduction in fibrosis would illustrate the connection between activation of iNKT cells using an iNKT activator and reduction of disease and disease markers such as pro-inflammatory factors. In this model, intratracheal instillation of the chemotherapeutic agent bleomycin induces epithelial damage, followed by infiltration of inflammatory senescent cells into the lung interstitium and alveolar space. Accumulation of senescent epithelial cells also occurs in lung tissue of patients with idiopathic lung fibrosis (IPF). See, Arora et al . . . , Med, vol 2, issue 8, 2021, 938-950.

Eight-to ten-week-old male C57BL/6 mice are intratracheally instilled with bleomycin (3U/kg) while under isoflurane anesthesia. 10 days post-injury, test compounds or vehicle control are intraperitoneal injected. To quantify target (INKT cell expansion) and mechanism engagement (senescent cell burden), lungs are harvested and processed into a single-cell suspension at day 14 (4 days post-treatment). Samples are stained to assess iNKT cells by via live cells (dapi-), CD3+, CD1d-aGalCer tetramer+ or senescent cells with live cell (dapi-) CD45, C12FDG^{high}, then run on a MACSquant 10 flow cytometer. Also at day 14, pro-fibrotic factors are measured in the bronchoalveolar lavage fluid (BALF). Post-necropsy lungs are flushed with chilled PBS. ELISA assays are performed on the BALF to assess for the change in pro-inflammatory factors TNFα and IL-17a. A hydroxyproline assay is performed to assess lung fibrosis at day 21 (11 days post-treatment). Lung samples are frozen, then mixed with 50% trichloroacetic acid after homogenization. The samples are incubated with 12N HCl at 110° C. overnight, then reconstituted in water and mixed with 1.4% chloramine T and 10% isopropanol and 0.5M sodium acetate. Absorbance is measured at 550 nm in Ehrlich's solution at 65° C. for 15 min.

Overview of the study design: Mice are injured at day 0 with bleomycin at 3U/kg, treated with iNKT activator or vehicle on day, then sacrificed either on day 14 to measure lung iNKT expansion, C₁₂FDG reduction via flowcytometry, or inflammatory factors. At day 21 mice are sacrificed 15 to measure lung fibrosis. All experiments are performed with n=3-10 animals per group. Comparison between different groups is done using one way ANOVA and statistical significance is given as P<0.05 *, P<0.001 ***, P<0.0001 ****

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A compound of formula (B): 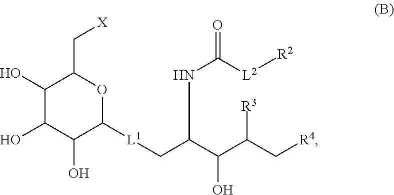
or a pharmaceutically acceptable salt thereof, wherein:
R² is cycloalkyl or heterocyclylalkyl, wherein the cycloalkyl or heterocyclylalkyl is optionally substituted with one or more R^{b};
R³ is hydrogen or —OH;
R⁴ is alkyl;
L¹ is —O— or —(CH₂)ₘ—;
L² is alkylene;
X is —OH, —OC(O)R⁷, —OC(O)NR⁹R¹⁰, or —N(R^{a})C(O)R¹³, wherein
when X is OH or —N(R^{a})C(O)R¹³, either
L¹ is —(CH₂)ₘ—, or
R³ is hydrogen;
R^{a} is, for each occurrence, independently hydrogen or alkyl;
R^{b} is, for each occurrence, independently oxo or halo;
m is 1 or 2;
R⁷ is alkyl optionally substituted with one or more R^{A};
each of R⁹ and R¹⁰ is independently hydrogen or cycloalkyl, wherein the cycloalkyl is optionally substituted with one or more halo;
R¹³ is cycloalkyl optionally substituted with one or more halo; and each R^{A} is independently aryl.

2. The compound of claim 1, or a pharmaceutically acceptable salt thereof, wherein the compound is a compound of formula (B-I): 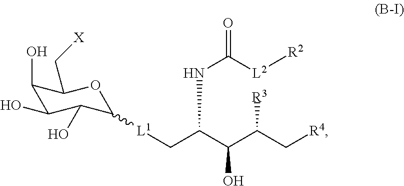
or a pharmaceutically acceptable salt thereof.

3. The compound of claim 1, or a pharmaceutically acceptable salt thereof, wherein the compound is a compound of formula (B-I-i) or (B-I-ii): 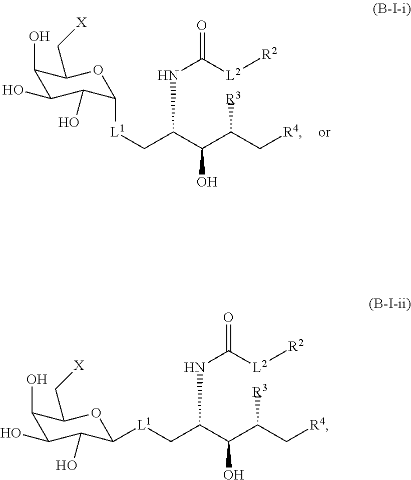
or a pharmaceutically acceptable salt thereof.

4. The compound of claim 1, or a pharmaceutically acceptable salt thereof, wherein the compound is a compound of formula (B-II): 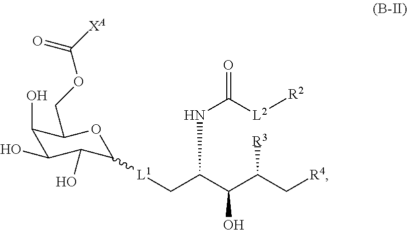
wherein X^{A} is —R⁷ or —NR⁹R¹⁰, or a pharmaceutically acceptable salt thereof.

5. The compound of claim 1, or a pharmaceutically acceptable salt thereof, wherein the compound is a compound of formula (B-III): 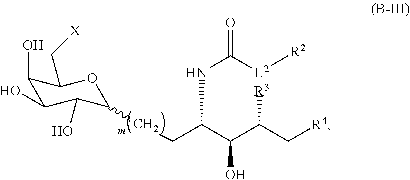
or a pharmaceutically acceptable salt thereof.

6. The compound of claim 1, or a pharmaceutically acceptable salt thereof, wherein R³ is —OH.

7. The compound of claim 1, or a pharmaceutically acceptable salt thereof, wherein the compound is a compound of formula (B-IV): 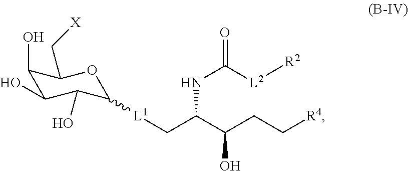
or a pharmaceutically acceptable salt thereof.

8. The compound of claim 1, or a
pharmaceutically acceptable salt thereof, wherein R² is

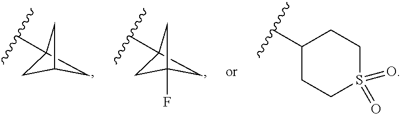 [SMILES: *C12CC(C1)C2 ; *C12CC(F)(C1)C2 ; *C1CCS(=O)(=O)CC1]

9. The compound of claim 1, or a pharmaceutically acceptable salt thereof, wherein R⁴ is C₁₃H₂₇.

10. The compound of claim 1, or a pharmaceutically acceptable salt thereof, wherein L¹ is —O—.

11. The compound of claim 1, or a pharmaceutically acceptable salt thereof, wherein L¹ is —(CH₂)ₘ— and m is 1.

12. The compound of claim 1, or a pharmaceutically acceptable salt thereof, wherein L² is —(CH₂) 23-.

13. The compound of claim 1, or a pharmaceutically acceptable salt thereof, wherein X is —OH.

14. The compound of claim 1, or a pharmaceutically acceptable salt thereof, wherein X is —OC(O)R⁷.

15. The compound of claim 14, or a pharmaceutically acceptable salt thereof, wherein R⁷ is

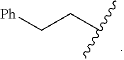 [SMILES: *CCc1ccccc1]

16. The compound of claim 1, or a pharmaceutically acceptable salt thereof, wherein X is —OC(O)NR⁹R¹⁰.

17. The compound of claim 16, or a pharmaceutically acceptable salt thereof, wherein one of R⁹ and R¹⁰ is hydrogen and the other of R⁹ and R¹⁰ is

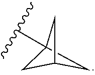 [SMILES: *C12CC(C1)C2]

18. The compound of claim 1, or a pharmaceutically acceptable salt thereof, wherein X is —N(R^{a})C(O)R¹³.

19. The compound of claim 18, or a pharmaceutically acceptable salt thereof, wherein one of R^{a} is hydrogen and R¹³ is

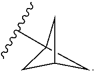 [SMILES: *C12CC(C1)C2]

20. (canceled)

21. The compound of claim 1, or a pharmaceutically acceptable salt thereof, wherein the compound is: 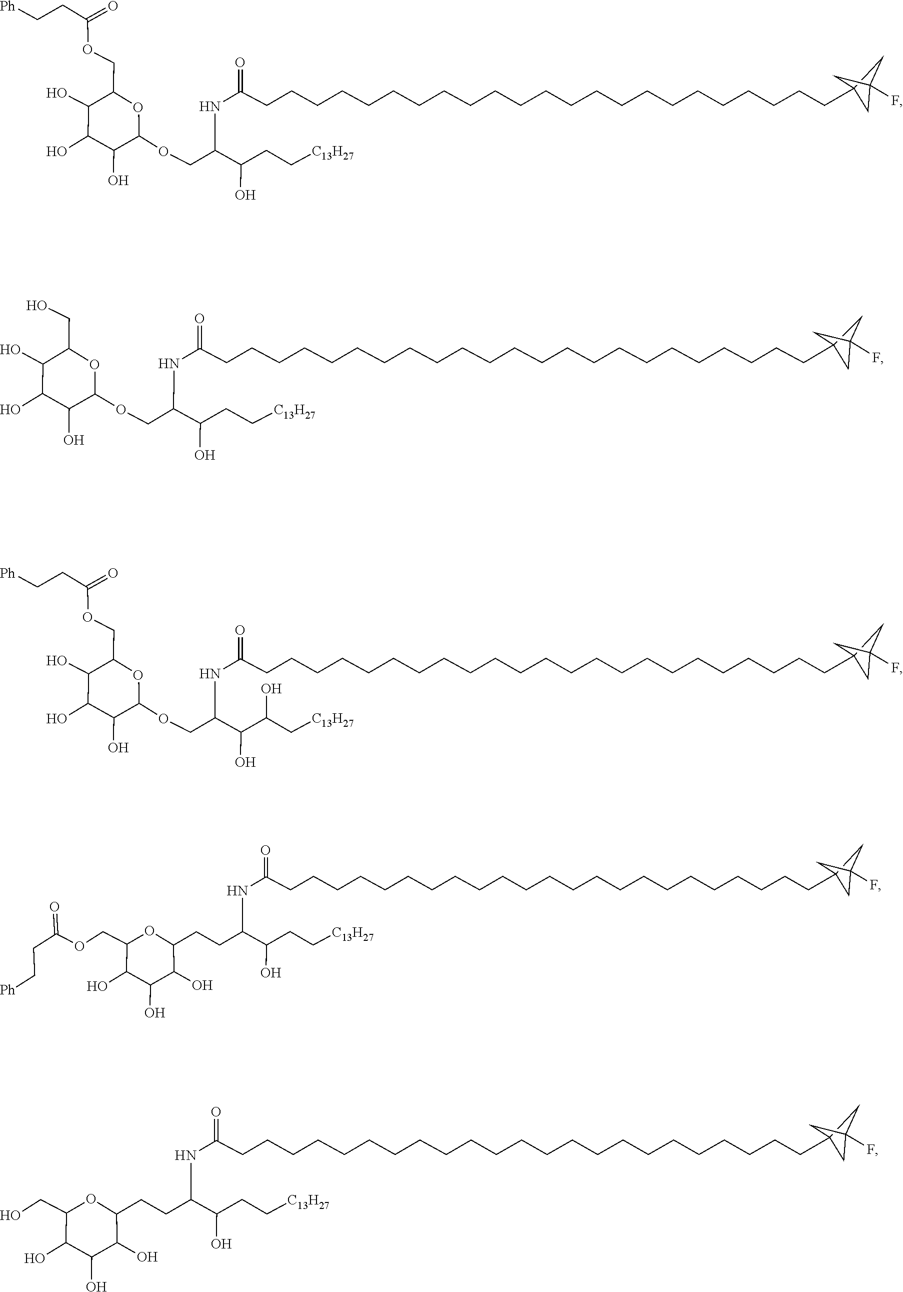 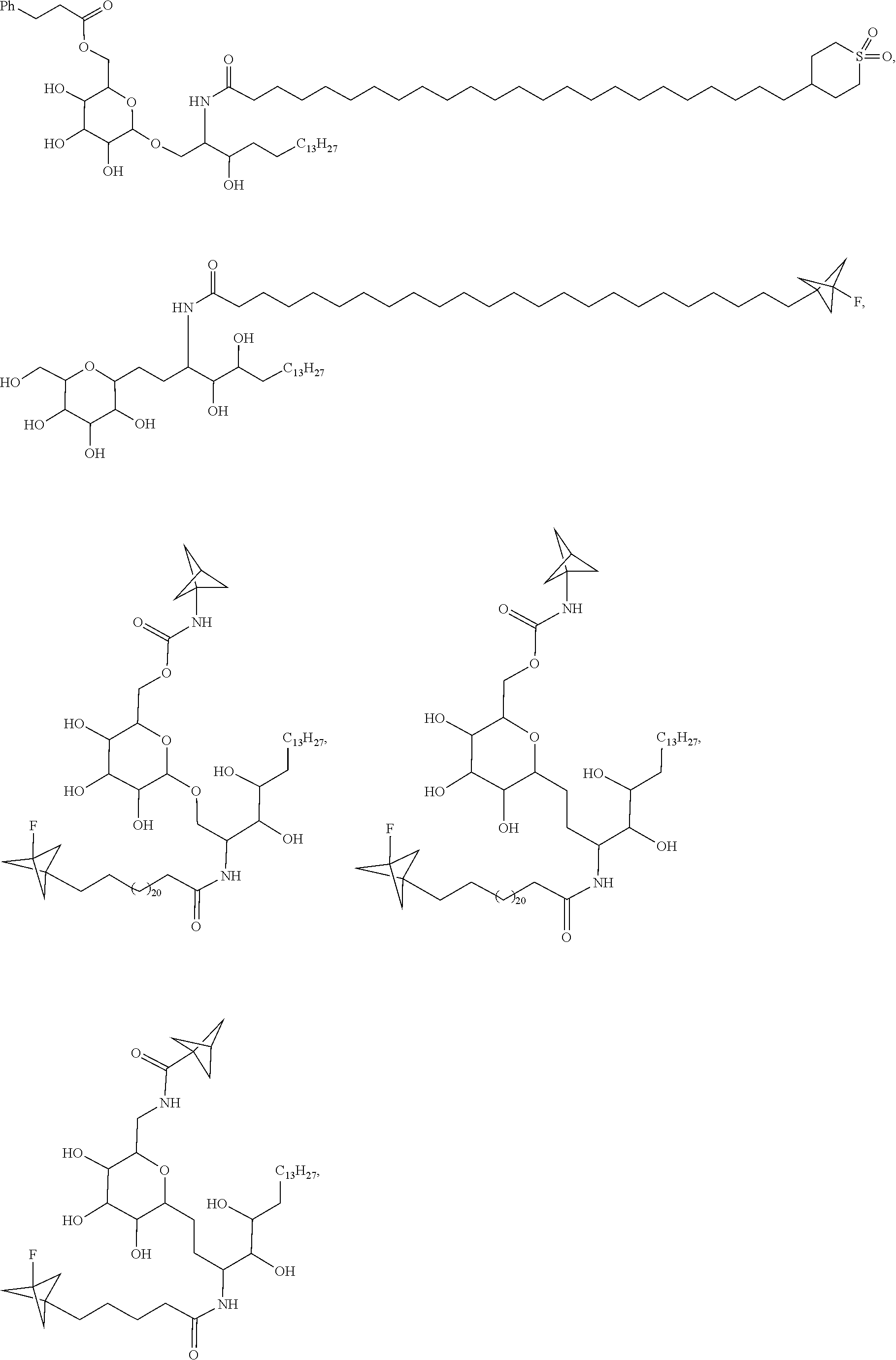 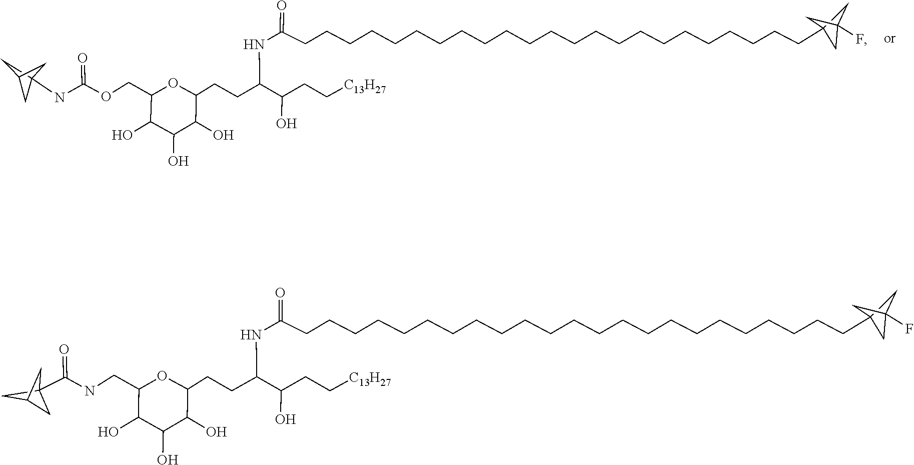
or a pharmaceutically acceptable salt thereof.

22. (canceled)

23. A pharmaceutical composition, comprising: a compound of claim 1; and at least one pharmaceutically acceptable carrier.

24. A method for activating invariant natural killer T (INKT) cells in a subject in need thereof, comprising: administering to the subject a compound of claim 1, or a pharmaceutical composition, comprising: a compound of claim 1; and at least one pharmaceutically acceptable carrier.

25. A method for selectively reducing the presence of or eliminating senescent cells by activating invariant natural killer T (INKT) cells in a subject in need thereof, comprising: administering to the subject a compound of claim 1, or a pharmaceutical composition, comprising: a compound of claim 1; and at least one pharmaceutically acceptable carrier.

26. A method of treating a disease, disorder or condition by activating invariant natural killer T (INKT) cells in a subject in need thereof, comprising: administering to the subject a compound of claim 1, or a pharmaceutical composition, comprising: a compound of claim 1; and at least one pharmaceutically acceptable carrier.

27. The method of claim 26, wherein the disease, disorder or condition is an autoimmune disease, an allergic disease, a metabolic syndrome or disorder, cancer, a pathogen infection, an eye disease, a disease of aging, fibrosis, heart disease, or kidney disease, or any combination thereof.

28. (canceled)

29. (canceled)

30. (canceled)

31. The method of claim 26, wherein the disease, disorder or condition is type 1 diabetes, type 2 diabetes, diabetic nephropathy, diabetic kidney disease, diabetic bladder dysfunction, diabetic retinopathy, diabetic macular edema, fatty liver disease, such as non-alcoholic fatty liver disease/metabolic-associated fatty liver disease (NAFLD/MAFLD), non-alcoholic steatohepatitis/metabolic dysfunction-associated steatohepatitis (NASH/MASH), cancer, an eye disease, age related macular degeneration, heart disease, heart failure, atherosclerosis, hypertension, kidney disease, cardiorenal syndrome, pathogen infection, rheumatoid arthritis, ulcerative colitis, multiple sclerosis, familial hypercholesteremia, giant cell arteritis, idiopathic pulmonary fibrosis (IPF), systemic lupus erythematosus, cachexia, Werner syndrome, Fuchs' endothelial dystrophy, glaucoma, cataracts, posterior non-infectious uveitis, chronic obstructive pulmonary disease, systemic sclerosis, pulmonary arterial hypertension, lipodystrophy, sarcopenia, osteoporosis, Duchenne muscular dystrophy, myotonic dystrophy, alopecia, post myocardial infarction, vitiligo, postural orthostatic tachycardia syndrome (POTS), medium-chain acyl-coenzyme A dehydrogenase deficiency (MCAD), Sjögren's syndrome, scleroderma, Hashimoto Disease, ankylosing spondylitis, fibromyalgia, sarcoidosis, hepatitis, Raynaud's Syndrome, chronic inflammatory response syndrome or mold illness, celiac, Crohn's disease, inflammatory bowel disease, pemphigus, Stiff Person Syndrome (SPS), Primary Biliary Cholangitis (PBC) (cholestatic diseases), psoriatic arthritis, chronic inflammatory demyelinating polyradiculoneuropathy (CIDP), motor neuron disease, granulomatosis with polyangiitis (GPA) (Wegener's disease), amyotrophic lateral sclerosis (ALS), primary open angle glaucoma (POAG), myasthenia gravis, or presbyopia, or any combination thereof.

32. (canceled)

33. (canceled)

34. (canceled)
